(19) United States
(12) Patent Application Publication
(10) Pub. No.: US 2026/0191893 A1
(43) Pub. Date: Jul. 9, 2026

(54) IDENTIFICATION OF MECHANOSENSING REGULATORS IN PLASMACYTOID DENDRITIC CELLS

(71) Applicants: Franck J. Barrat, Eastchester, NY (US); Vidyanath Chaudhary, New York, NY (US)

(72) Inventors: Franck J. Barrat, Eastchester, NY (US); Vidyanath Chaudhary, New York, NY (US)

(21) Appl. No.: 19/346,261

(22) Filed: Sep. 30, 2025

Related U.S. Application Data
(60) Provisional application No. 63/702,032, filed on Oct. 1, 2024.

(51) Int. Cl. A61K 31/704; A61K 31/195; A61K 31/403; A61K 31/473; A61K 31/565; A61K 31/585; A61K 38/10; A61P 17/00

(52) U.S. Cl. CPC A61K 31/704; A61K 31/195; A61K 31/403; A61K 31/473; A61K 31/565; A61K 31/585; A61K 38/10; A61P 17/00

(57) ABSTRACT

The present application is directed to mechanosensing regulators that are useful in the treatment of various diseases.

### CLAIM OF PRIORITY

This application claims priority to U.S. Provisional Application Ser. No. 63/702,032, filed on Oct. 1, 2024, the entire contents of which are hereby incorporated by reference.

### SEQUENCE LISTING

This application contains a Sequence Listing that has been submitted electronically as an XML file named 27601-0093001_SL_ST26.xml. The XML file, created on Mar. 6, 2026, is 25,598 bytes in size. The material in the XML file is hereby incorporated by reference in its entirety.

### FIELD OF THE INVENTION

The present application is directed to mechanosensing regulators that are useful in the treatment of various diseases.

### BACKGROUND OF THE INVENTION

Monocytes have crucial roles during immune responses to pathogens but can also promote autoimmune and fibrotic diseases. The inflammatory response in tissues such as the skin can be triggered by tissue damage due to infection or injury^{1,2}. The initial phase is characterized by an influx in the tissue of neutrophils and monocytes which is associated with the expression of proinflammatory mediators including cytokines and chemokines. These then favor the infiltration of immune cells which help eliminate pathogens and promote tissue repair and remodeling^{1,3-6}. During inflammation, monocytes circulate through the bloodstream and extravasate into inflamed tissues⁷⁻¹⁰. Under these conditions, monocytes acquire unique and essential functions, including proinflammatory activities, antigen presentation, tissue remodeling, and anti-inflammatory roles, that often cannot be fulfilled by resident macrophages and conventional dendritic cells^{10,11}. This is supported by depleting strategies which document the critical role played by monocytes during inflammation in promoting tissue repair, or alternatively, contributing to development of autoimmunity and fibrosis^{8,10,12,13}. To maintain homeostasis of cellular functions, effective coordination of various biological and mechanical signals within tissues is essential¹⁴. Signals originating from mechanical properties, including the stiffness of the extracellular matrix (ECM), also referred to as mechanosensing, influence cellular pathways and functions¹⁴⁻¹⁶. The significance of mechanical stimuli has long been recognized in neuron functioning, developmental biology, and within organ systems, such as the cardiovascular and skeletal systems^{15,17,18}. New data recently highlighted the potential effect of PIEZO1 or YAP/TAZ-TEAD mediated mechanosensing in regulating the activation of immune cells^{15,19}. However, our current understanding of immune cell activation in tissue remains limited and mostly related to how PAMPs (pathogen-associated molecular patterns) and DAMPs (danger/damage-associated molecular patterns) released in the tissue injury trigger cells during infections or autoimmunity. Yet, when immune cells infiltrate the skin, they invariably encounter the mechanical properties attributable to tissue stiffness. Stiffness in human organs is lowest in the brain and liver (0.2-0.5 kPa) and highest in the bones (up to 15,000 kPa)^{20,21}, while cells in circulation only experience a pressure of ~0.01 kPa. The stiffness of the skin is also significantly higher than that of blood and is estimated to apply 1-2 kPA pressure on the cells²¹⁻²³, while in fibrotic skin, this can rise to 25-50 kPA²¹⁻²³. Furthermore, the stiffness of skin increases during inflammation and wound healing^{22,23}. However, the precise impact of mechanosensing on monocyte function remains elusive. This disclosure shows that skin-infiltrating monocytes can sense the stiffness in the skin and induce mechanosensing and inflammatory genes, including IL-1β, IL-6, and TNF-α. Mechanistically, mechanosensing promotes the binding of the transcription factor (TF) SP1 to the promoters of inflammatory genes, thereby amplifying the expression of proinflammatory genes upon TLR activation. Additionally, mechanosensing selectively regulated monocytes of SSc (systemic sclerosis) patients in the high-stiffness microenvironment of fibrotic skin, blocking a feedback mechanism that promotes wound healing, and leading to chronic inflammation. Overall, the findings disclosed herein reveal a novel mechanism of mechanosensing in monocytes and provide new insights into their regulation in the context of skin injury and in a fibrotic disease.

Most autoimmune immune diseases have no standard medical treatments and have very few approved drugs for medical uses. In autoimmune diseases, monocytes/macrophages are some of the most important immune cells playing a key role in diseases, as they are capable of secreting abundant amount of inflammatory cytokines such as TNF-α, IL-1β, and IL-6. Monocytes infiltrate into the skin leading to chronic inflammation and fibrotic condition. The stiffness or physical pressure of skin is 200 times higher than blood and little is known on the impact of high stiffness on monocytes/macrophages response.

The data disclosed herein show that the increase of stiffness activates monocytes and induces inflammatory mediators such as cytokines or chemokines. The mechanism by which stiffness or physical pressure by skin in both normal or fibrotic conditions impacts macrophages is also unknown and the data disclosed herein uncover new pathways underlying this effect. We have targeted these pathways in vitro and validated their involvement in vivo, The newly identified involvement of the pathways in activating macrophages activation leads to the discovery of several new targets for drugs development to meet the unmet medical need of treatments for autoimmune diseases.

### SUMMARY

Provided herein is a method of inhibiting mechanosensing in monocytes, said method comprising inhibition of the SP1-mediated mechanosensing pathway.

Some embodiments provide a method of inhibiting the SP1-mediated mechanosensing pathway, said method comprising inhibition of SP1 transcription factor.

Some embodiments provide a method of inhibiting the SP1-mediated mechanosensing pathway, said method comprising antagonism of the TREM1 receptor.

Some embodiments provide a method of inhibiting the SP1-mediated mechanosensing pathway, said method comprising antagonism of the GPER1 receptor.

Some embodiments provide a method of inhibiting the SP1-mediated mechanosensing pathway, said method comprising antagonism of the GPR30 receptor.

In some embodiments, the monocytes are in skin tissue.

Also provided herein is a method of treating a disease or disorder associated with inhibiting mechanosensing in monocytes, said method comprising inhibition of the SP1-mediated mechanosensing pathway.

Some embodiments provide a method of treating a disease or disorder associated with inhibiting the SP1-mediated mechanosensing pathway, said method comprising inhibition of SP1 transcription factor.

Some embodiments provide a method of treating a disease or disorder associated with inhibiting the SP1-mediated mechanosensing pathway, said method comprising antagonism of the TREM1 receptor.

Some embodiments provide a method of treating a disease or disorder associated with inhibiting the SP1-mediated mechanosensing pathway, said method comprising antagonism of the GPER1 receptor.

Some embodiments provide a method of treating a disease or disorder associated with inhibiting the SP1-mediated mechanosensing pathway, said method comprising antagonism of the GPR30 receptor.

In some embodiments, the disease or disorder is an autoimmune disorder or a fibrotic disorder.

In some embodiments, the disease or disorder is a skin disease or disorder.

### BRIEF DESCRIPTION OF THE FIGURES

FIG. 1A-1, FIG. 1A-2, FIG. 1A-3, FIG. 1A-4, and FIG. 1A-5 show that skin injury triggers an inflammatory response in infiltrating monocytes as noted in Example 1. 8-10 weeks old C57BL/6 mice were either shaved only or tape stripped (TAPE) to provoke inflammation. After day 1, skin biopsies were collected. Total RNA of skin were isolated from skin biopsies and analyzed for inflammatory genes such as TNF-α, IL-1β, IL-1α, IP-10 and IL-6 expression. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 1B shows that skin injury induces the infiltration of immune cells in the skin as noted in Example 1. 8-10 weeks old C57BL/6 mice were either shaved only or tape stripped (TAPE) to provoke inflammation. After day 1, skin biopsies were collected. Infiltration of CD45⁺ cell in skin was analyzed via flow cytometer. Individual mice are indicated; all results are represented as mean SEM; and statistical significance was evaluated using Mann-Whitney U test or one-way ANOVA, Tukey's multiple comparisons test. ns, P>0.05; *, P<0.05; ***, P<0.001.

FIG. 1C shows that skin injury induces the infiltration of neutrophils in the skin as noted in Example 1. 8-10 weeks old C57BL/6 mice were either shaved only or tape stripped (TAPE) to provoke inflammation. After day 1, skin biopsies were collected. Infiltration of neutrophils (CD45⁺CD11b⁺Ly6G⁺) in skin was analyzed via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; ***P<0.001.

FIG. 1D shows that skin injury induces the infiltration of macrophages in the skin 1 as noted in Example 1. 8-10 weeks old C57BL/6 mice were either shaved only or tape stripped (TAPE) to provoke inflammation. After day 1, skin biopsies were collected. Infiltration of macrophages (CD45⁺CD11b⁺Ly6G⁻F480⁺) in skin was analyzed via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 1E shows that skin injury induces the infiltration of inflammatory monocytes in the skin as noted in Example 1. 8-10 weeks old C57BL/6 mice were either shaved only or tape stripped (TAPE) to provoke inflammation. After day 1, skin biopsies were collected. Infiltration of inflammatory monocytes (CD45⁺CD11b⁺Ly6G⁻Ly6C⁺) in skin was analyzed via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 1F shows that skin injury induces the infiltration of monocyte subsets in the skin 1 as noted in Example 1. 8-10 weeks old C57BL/6 mice were either shaved only or tape stripped (TAPE) to provoke inflammation. After day 1, blood and skin biopsies were collected. Staining of infiltrating monocytes (CD45⁺CD11b⁻Ly6G⁻Ly6C⁺CCR2^{low/high}) in blood and skin was analyzed via flow cytometer.

FIG. 1G shows that skin injury induces the infiltration of monocyte subsets in the skins as noted in Example 1. 8-10 weeks old C57BL/6 mice were either shaved only or tape stripped (TAPE) to provoke inflammation. After day 1, blood and skin biopsies were collected. Infiltration of infiltrating monocytes (CD45⁺CD11b⁻Ly6G⁻Ly6C⁺CCR2^{low/high}) in blood and skin was analyzed via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 1H shows that following skin injury, infiltrating monocytes express the activation marker CD861 as noted in Example 1. 8-10 weeks old C57BL/6 mice were either shaved only or tape stripped (TAPE) to provoke inflammation. After day 1, blood and skin biopsies were collected. CD86 expression on infiltrating monocytes in blood and skin was analyzed via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 1I-1, FIG. 1I-2, FIG. 1I-3, and FIG. 1I-4 show that following skin injury, infiltrating monocytes express inflammatory genes as noted in Example 1. 8-10 weeks old C57BL/6 mice were either shaved only or tape stripped (TAPE) to provoke inflammation. After day 1, blood and skin biopsies were collected. Infiltrating monocytes were sorted from blood or skin and collected for RNA. Gene expressions for inflammatory cytokines such as IL-1β, IL-6, TNF-α, and CXCL2 were analyzed. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2A shows that stiffness-mediated mechanosensing triggers inflammatory responses in human monocytes as noted in Example 2. Monocytes from freshly isolated blood of HDs were cultured at a stiffness of 0 kPa, 0.2 kPa, 2 kPa or 50 kPa for 24 h. Secretion of cytokines such as IL-1β was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2B shows that stiffness-mediated mechanosensing triggers inflammatory responses in human monocytes as noted in Example 2. Monocytes from freshly isolated blood of HDs were cultured at a stiffness of 0 kPa, 0.2 kPa, 2 kPa or 50 kPa for 24 h. Secretion of cytokines such as TNF-α was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2C shows that stiffness-mediated mechanosensing triggers inflammatory responses in human monocytes as noted in Example 2. Monocytes from freshly isolated blood of HDs were cultured at a stiffness of 0 kPa, 0.2 kPa, 2 kPa or 50 kPa for 24 h. Secretion of cytokines such as IL-6 was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2D shows that stiffness-mediated mechanosensing triggers inflammatory responses in human monocytes as noted in Example 2. Monocytes from freshly isolated blood of HDs were cultured at a stiffness of 0 kPa, 0.2 kPa, 2 kPa or 50 kPa for 24 h. CD86 expression was measured by flow cytometry. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2E shows that stiffness-mediated mechanosensing triggers inflammatory responses in human monocytes which can potentiate the response by the cells to additional stimuli as noted in Example 2. Monocytes were cultured at a stiffness of either 0 kPa or 2 kPa for 24 h, then washed with fresh media. Cells were then cultured with either TLR4 ligand (LPS:10 ng/ml) or TLR8 ligand (ORN8L:130 μg/ml) for 6 h and secretion of IL-1β was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2F shows that stiffness-mediated mechanosensing triggers inflammatory responses in human monocytes which can potentiate the response by the cells to additional stimuli as noted in Example 2. Monocytes were cultured at a stiffness of either 0 kPa or 2 kPa for 24 h, then washed with fresh media. Cells were then cultured with either TLR4 ligand (LPS:10 ng/ml) or TLR8 ligand (ORN8L:130 μg/ml) for 6 h and secretion of IL-6 was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2G shows that stiffness-mediated mechanosensing triggers inflammatory responses in human monocytes which can potentiate the response by the cells to additional stimuli as noted in Example 2. Monocytes were cultured at a stiffness of either 0 kPa or 2 kPa for 24 h, then washed with fresh media. Cells were then cultured with either TLR4 ligand (LPS:10 ng/ml) or TLR8 ligand (ORN8L:130 μg/ml) for 6 h and secretion of TNF-α was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 2H shows that stiffness-mediated mechanosensing triggers inflammatory responses in human monocytes as noted in Example 2. Monocytes from freshly isolated blood of healthy donors (HDs) were cultured at a stiffness of either 0 kPa, 0.2 kPa or 50 kPa for 24 h and analyzed by RNA-sequencing. Heatmaps showing the inflammatory cytokines.

FIG. 21 shows that stiffness-mediated mechanosensing triggers inflammatory responses in human monocytes as noted in Example 2. Monocytes from freshly isolated blood of healthy donors (HDs) were cultured at a stiffness of either 0 kPa, 0.2 kPa or 50 kPa for 24 h and analyzed by RNA-sequencing. All DEGs were analyzed for receptors that are impacted by change in stiffness using QIAGEN Ingenuity Pathway Analysis (IPA). Red color indicates activated receptor and blue color indicated inhibited receptor.

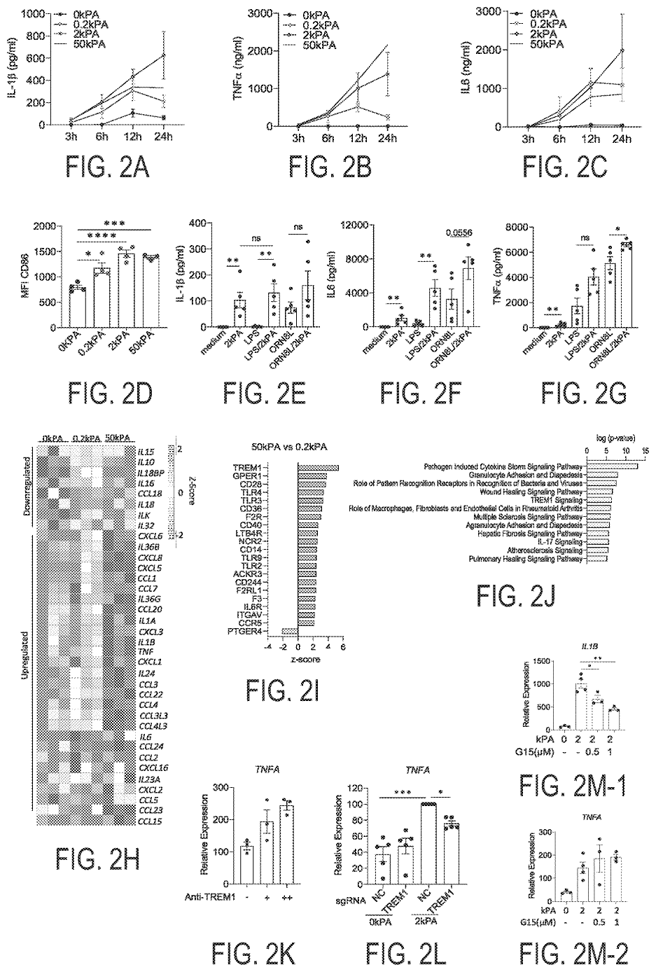

FIG. 2J shows that stiffness-mediated mechanosensing triggers inflammatory responses in human monocytes as noted in Example 2. Monocytes from freshly isolated blood of healthy donors (HDs) were cultured at a stiffness of either 0 kPa, 0.2 kPa or 50 kPa for 24 h and analyzed by RNA-sequencing. All DEGs were analyzed for pathways that are impacted by change in stiffness using QIAGEN Ingenuity Pathway Analysis (IPA).

FIG. 2K shows that TREM1 signaling can induce the activation of human monocytes as noted in Example 2. Monocytes were cultured in media alone or with antibody for TREM1 (5 or 10 μg/ml) for 6 h. Secretion of TNF-α was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***P<0.001.

FIG. 2L shows that stiffness-mediated mechanosensing triggers inflammatory responses in human monocytes through TREM1 signaling as noted in Example 2. Monocytes were transfected with CRISPR-Cas9 complex targeting for TREM1 using Lipofectamine™ CRISPRMAX reagent. After transfection, monocytes were cultured with baricitinib (1 μM) for 24 h, followed by culturing in fresh media for 48 h, and then subjected to either 0 kPa or 2 kPa stiffness for 6 h. RNA were collected and analyzed for the expression of TNF-α by Q-PCR. Individual donors are indicated; all results are represented as mean±SEM; and statistical significance was evaluated using Mann-Whitney U test or one-way ANOVA, Tukey's multiple comparisons test. ns, P>0.05; *, P<0.05; ***P<0.001.

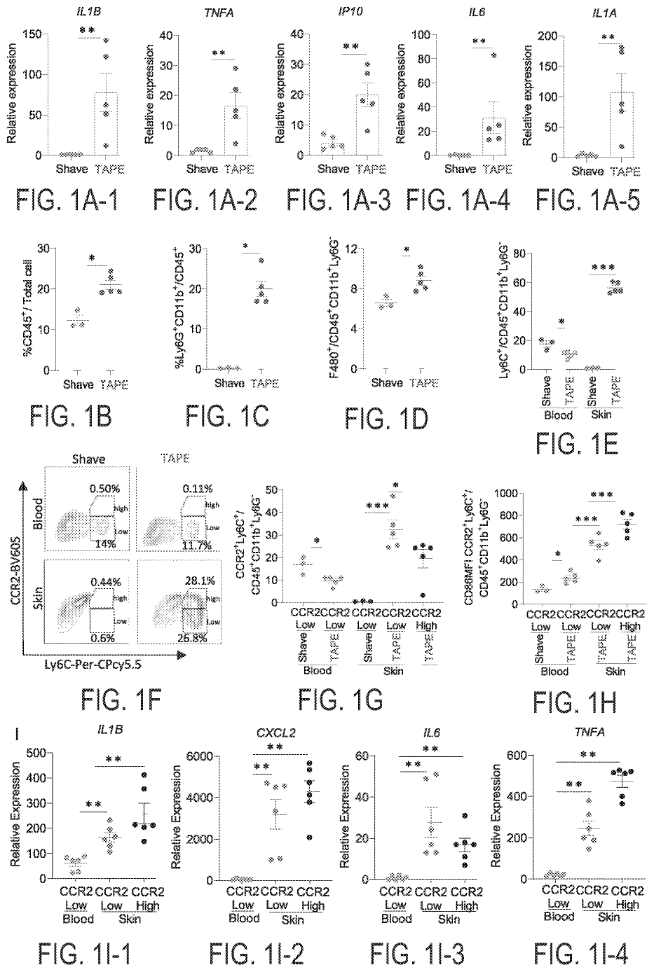

FIG. 2M-1 and FIG. 2M-2 show that stiffness-mediated mechanosensing triggers inflammatory responses in human monocytes through the GPR30 receptors noted in Example 2. Monocytes were cultured in media alone or with selective GPR30 inhibitor (G15) for 1 h, followed by culturing in either 0 kPA or 2 kPA stiffness for 6 h. RNA were collected and analyzed for the expression of TNF-α and IL-1β/by Q-PCR. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 3A shows that stiffness induces epigenetic changes in monocytes as noted in Example 3. Monocytes from freshly isolated blood of HDs were cultured at a stiffness of either 0.2 kPa or 50 kPa for 6 h and ATAC-sequencing was performed. PCA plot of ATAC-seq analysis.

FIG. 3B shows that that stiffness-mediated mechanosensing promotes the binding of the transcription factor SP1 to the promoter of inflammatory genes in monocytes as noted in Example 3. Monocytes from freshly isolated blood of HDs were cultured at a stiffness of either 0.2 kPa or 50 kPa for 6 h and ATAC-sequencing was performed. Enrichment heatmaps of normalized chromatin accessibility reads of ATAC-seq at 0.2 kPa and 50 kPa stiffness. The top lines represent the peak signal of each condition at 5′cut site. White and red indicate the min and max of the average signal of four replicates.

FIG. 3C shows that stiffness is associated with differential peaks in ATAC-sequencing.-mediated mechanosensing promotes the binding of the transcription factor SP1 to the promoter of inflammatory genes in monocytes as noted in Example 3. Monocytes from freshly isolated blood of HDs were cultured at a stiffness of either 0.2 kPa or 50 kPa for 6 h and ATAC-sequencing was performed. De novo motif analysis of differential peaks at various stiffness using HOMER.

FIG. 3D shows that stiffness-mediated induces transcriptional changes in monocytes as noted in Example 3. Monocytes were cultured at a stiffness of either 0.2 kPa or 50 kPa and analyzed by RNA-sequencing. All differentially expressed genes (DEGs) were analyzed for activation/inhibition of transcription factors (TFs) using QIAGEN Ingenuity Pathway Analysis (IPA). Red color indicates activation and blue color indicates inhibition.

FIG. 3E-1 and FIG. 3E-2 show stiffness-mediated binding sites for SP1 and NYFA as noted in Example 3. Monocytes from freshly isolated blood of HDs were cultured at a stiffness of either 0.2 kPa or 50 kPa for 6 h and ATAC-sequencing was performed. JASPAR motif cut site analysis of the transcription factors NFYA and SP1 was done using HSS Genomics R scripts.

FIG. 3F shows that stiffness-mediated mechanosensing identifies SP1 as a key transcription factor associated with monocyte inflammation as noted in Example 3. Monocytes were cultured at a stiffness of either 0.2 kPa or 50 kPa and analyzed by RNA-sequencing. All differentially expressed genes (DEGs) were analyzed for transcription factor network with transcription factor SP1. Red color indicates activation and blue color indicates inhibition.

FIG. 3G-1, FIG. 3G-2, and FIG. 3G-3 show that stiffness-mediated mechanosensing requires SP1 as noted in Example 3. Monocytes were transfected with CRISPR-Cas9 complex targeting for SP1 on using Lipofectamine™ CRISPRMAX reagent. After transfection, monocytes were cultured for 72 h, followed by 2 kPA stiffness for 6 h. RNA were collected and analyzed for TNF-α and IL-1p expression. Secretion of TNF-α was analyzed by ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 3H-1 and FIG. 3H-2 show that stiffness-mediated mechanosensing requires SP1 as noted in Example 3. Monocytes were cultured in media alone or with selective SP1 inhibitor (Mith: mithramycin) for 1 h, followed by cultured in either 0 kPA or 2 kPA stiffness for 6 h. RNA were collected and analyzed for the expression of TNF-α and IL-1p by Q-PCR. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 3I-1, FIG. 3I-2, and FIG. 3I-3 show that stiffness-mediated mechanosensing promotes the binding of the transcription factor SP1 to the promoter of inflammatory genes in monocytes as noted in Example 3. Monocytes were cultured at a stiffness of either 0.2 kPa or 50 kPa for 18 h and analyzed by CUT&RUN assay. Track changes showing the binding of SP1 at the peak of chromatin opening observed using IGV. Representative average signal tracks showing IL-1β, TNF-α, and IL-10 loci from CUT&RUN assay and ATAC-seq analysis.

FIG. 4A shows PCA plot analysis of RNA-seq analysis of monocyte subsets infiltrating the skin after tape stripping as noted in Example 4. 8-10 weeks old C57BL/6 mice were either shaved only or tape stripped (TAPE) to provoke inflammation. At day 1, infiltrating monocytes (CD45⁺CD11b⁻Ly6G⁻ Ly6C⁺CCR2^{+(low/high)}) were FACS-sorted from the blood or inflamed skin biopsies. FACS-sorted infiltrating monocytes were analyzed by RNA-sequencing. PCA plot of RNA-seq analysis.

FIG. 4B shows the heatmap of genes regulated in monocyte subsets infiltrating the skin after tape stripping as noted in Example 4. 8-10 weeks old C57BL/6 mice were either shaved only or tape stripped (TAPE) to provoke inflammation. At day 1, infiltrating monocytes (CD45⁺CD11b⁻Ly6G⁻ Ly6C⁺CCR2^{+(low/high)}) were FACS-sorted from the blood or inflamed skin biopsies. FACS-sorted infiltrating monocytes were analyzed by RNA-sequencing. Heatmaps showing inflammatory cytokines in infiltrating monocytes from the blood and inflamed skin biopsies.

FIG. 4C shows pathway analysis comparing monocyte subsets infiltrating the skin after tape stripping as noted in Example 4. 8-10 weeks old C57BL/6 mice were either shaved only or tape stripped (TAPE) to provoke inflammation. At day 1, infiltrating monocytes (CD45⁺CD11b⁻Ly6G⁻Ly6C⁺CCR2^{+(low/high)}) were FACS-sorted from the blood or inflamed skin biopsies. FACS-sorted infiltrating monocytes were analyzed by RNA-sequencing. All DEGs were analyzed for pathways that are impacted by change in stiffness from blood to skin using QIAGEN Ingenuity Pathway Analysis (IPA). Red color indicates activated pathway and blue color indicated inhibited pathway.

FIG. 4D shows various receptors differentially regulated between skin and blood monocytes following tape stripping as noted in Example 4. 8-10 weeks old C57BL/6 mice were either shaved only or tape stripped (TAPE) to provoke inflammation. At day 1, infiltrating monocytes (CD45⁺CD11b⁻Ly6G⁻ Ly6C⁺CCR2^{+(low/high)}) were FACS-sorted from the blood or inflamed skin biopsies. FACS-sorted infiltrating monocytes were analyzed by RNA-sequencing. All DEGs were analyzed for receptors that are impacted by change in stiffness from blood to skin using QIAGEN Ingenuity Pathway Analysis (IPA). Red color indicates activated receptor and blue color indicated inhibited receptor.

FIG. 4E-1 and FIG. 4E-2 show analysis of the expression of inflammatory genes in monocyte subsets from blood versus skin after tape stripping as noted in Example 4. 8-10 weeks old C57BL/6 mice were administrated intraperitoneally with either PBS or Mithramycin-A (0.5 mg/kg) for 3 consecutive days. Mice were either shaved only or tape stripped (TAPE) to provoke inflammation. After 24 h, blood and skin biopsies were collected. Infiltrating monocytes (CD45⁺CD11b⁻Ly6G⁻ Ly6C⁺CCR2^{+(low/high)}) were FACS-sorted from the blood or skin biopsies and gene expressions for inflammatory cytokines such as IL-1β, IL-6, TNF-α, and CXCL2 were analyzed. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 4F shows the infiltration in the skin of neutrophils following tape stripping as noted in Example 4. 8-10 weeks old C57BL/6 mice were administrated intraperitoneally with either PBS or Mithramycin-A (0.5 mg/kg) for 3 consecutive days. Mice were either shaved only or tape stripped (TAPE) to provoke inflammation. After 24 h, skin biopsies were collected for flow cytometer and analyzed the infiltration of neutrophils. This is a representative graph of one mouse.

FIG. 4G-1, FIG. 4G-2, and FIG. 4G-3 show the infiltration in the skin of neutrophils following tape stripping as noted in Example 4. 8-10 weeks old C57BL/6 mice were administrated intraperitoneally with either PBS or Mithramycin-A (0.5 mg/kg) for 3 consecutive days. Mice were either shaved only or tape stripped (TAPE) to provoke inflammation. After 24 h, skin biopsies were collected for flow cytometer and analyzed for the infiltration of neutrophils. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 4H-1, FIG. 4H-2, FIG. 4H-3, and FIG. 4H-4 show the expression of inflammatory genes in skin biopsies in mice after tape stripping and the presence of Mithramycin-A as noted in Example 4. 8-10 weeks old C57BL/6 mice were administrated intraperitoneally with either PBS or Mithramycin-A (0.5 mg/kg) for 3 consecutive days. Mice were either shaved only or tape stripped (TAPE) to provoke inflammation. After 24 h, skin biopsies were collected and analyzed for the expression of inflammatory genes such as TNF-α, IL-1B, IP-10 and IL-6. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 5A shows that monocytes from the skin of SSc patients differ from those in HDs as noted in Example 5. Single cell RNA-sequencing (sc-RNA-seq) were re-analyzed for infiltrating monocytes from published dataset (Gur et al., 2022, Cell; doi: 10.1016/j.cell.2022.03.011). U-map plot of monocytes infiltration into skin from 56 healthy donors and 97 SSc patients.

FIG. 5B-1, FIG. 5B-2, FIG. 5B-3, and FIG. 5B-4 show the expression of inflammatory genes in skin monocytes comparing SSc versus HDs as noted in Example 5. Single cell RNA-sequencing (sc-RNA-seq) were re-analyzed for infiltrating monocytes from published dataset (Gur et al., 2022, Cell; doi: 10.1016/j.cell.2022.03.011). Expression of inflammatory genes such as TNF-α, IL-1β, IL-6, CCL4, and CXCL8 in infiltrating monocytes from blood or skin of SSc patients.

FIG. 5C shows that mechanosensing differentially impacts the monocytes from SSc patients as noted in Example 5. Monocytes from freshly isolated blood of healthy donors (HDs) or SSc patients were cultured at a stiffness of either 0 kPa, 0.2 kPa or 50 kPa and analyzed by RNA-sequencing. PCA plot of RNA-seq analysis.

FIG. 5D shows that mechanosensing differentially impacts the monocytes from SSc patients as noted in Example 5. Monocytes from freshly isolated blood of healthy donors (HDs) or SSc patients were cultured at a stiffness of either 0 kPa, 0.2 kPa or 50 kPa and analyzed by RNA-sequencing. Heatmaps of the inflammatory cytokines that are impacted by stiffness.

FIG. 5E shows that mechanosensing differentially impacts the monocytes from SSc patients as noted in Example 5. Monocytes from freshly isolated blood of healthy donors (HDs) or SSc patients were cultured at a stiffness of either 0 kPa, 0.2 kPa or 50 kPa and analyzed by RNA-sequencing. All DEGs were analyzed for receptors that are impacted in HD and SSc at 0 kPA using QIAGEN Ingenuity Pathway Analysis (IPA). Red color indicates activation and blue color indicates inhibition.

FIG. 5F shows that mechanosensing activates distinct genes in inflammatory monocytes of SSc to promote chronic inflammatory response as noted in Example 5. Monocytes from freshly isolated blood of healthy donors (HDs) or SSc patients were cultured at a stiffness of either 0 kPa, 0.2 kPa or 50 kPa and analyzed by RNA-sequencing. Venn diagram of all DEGs that are impacted by change in stiffness when monocytes isolated HD vs SSc were cultured at 0 kPA, 0.2 kPA and 50 kPA.

FIG. 5G shows that mechanosensing differentially impacts the monocytes from SSc patients as noted in Example 5. Monocytes from freshly isolated blood of healthy donors (HDs) or SSc patients were cultured at a stiffness of either 0 kPa, 0.2 kPa or 50 kPa and analyzed by RNA-sequencing. Heatmaps of 70 genes from the Venn diagram FIG. 5F.

FIG. 5H shows that mechanosensing differentially impacts the monocytes from SSc patients as noted in Example 5. Monocytes were transfected with CRISPR-Cas9 complex targeting for VAV3 using Lipofectamine™ CRISPRMAX reagent. After transfection, monocytes were cultured with baricitinib (1 μM) for 24 h, followed by culturing with fresh media for 48 h, and then culturing at 0.2 kPA stiffness for 6 h. RNA were collected and analyzed for VAV3 expression.

FIG. 5I shows that mechanosensing differentially impacts the monocytes from SSc patients as noted in Example 5. Monocytes were transfected with CRISPR-Cas9 complex targeting for VAV3 using Lipofectamine™ CRISPRMAX reagent. After transfection, monocytes were cultured with baricitinib (1 μM) for 24 h, followed by culturing with fresh media for 48 h, and then culturing at 0.2 kPA stiffness for 6 h. RNA were collected and analyzed for IL-1p expression.

FIG. 5J-1 and FIG. 5J-2 show that VaV3 is involved in mechanosensing in monocytes as noted in Example 5. Monocytes were transfected with CRISPR-Cas9 complex targeting for VAV3 using Lipofectamine™ CRISPRMAX reagent. After transfection, monocytes were cultured with baricitinib (1 μM) for 24 h, followed by culturing with fresh media for 48 h, and then culturing at 0.2 kPA stiffness for 6 h. RNA were collected and analyzed for CXCL8 expression. Individual donors are indicated; all results are represented as mean±SEM; and statistical significance was evaluated using Mann-Whitney U test or one-way ANOVA, Tukey's multiple comparisons test. ns, P>0.05; * P<0.05; ***, P<0.001.

FIG. 6 shows gating of immune cell infiltration in Tapes tripping mice as noted in Example 6. 8-10 weeks old C57BL/6 mice were tape stripped (TAPE). After day 1, blood and skin biopsies were collected. Graphical representation of gating strategy for infiltration of immune cell: neutrophils (CD45⁺CD11b⁺Ly6G⁺), inflammatory monocytes (CD45⁺CD11b⁺Ly6G⁻Ly6C⁺), and infiltrating monocytes (CD45⁺CD11b⁻Ly6G⁻Ly6C⁺CCR2^{low/high}) in inflamed skin via flow cytometer.

FIG. 7A-1, FIG. 7A-2, and FIG. 7A-3 show that stiffness-mediated mechanosensing triggers inflammatory responses in monocytes as noted in Example 7. Monocytes from freshly isolated blood of HDs were cultured at a stiffness of 0 kPa, 0.2 kPa, 2 kPa or 50 kPa for 24 h. RNA was collected and analyzed for the expression of IL-6, TNF-α, and IL-1β.

FIG. 7B-1 and FIG. 7B-2 show that stiffness-mediated mechanosensing impacts gene expression in monocytes as noted in Example 7. Monocytes from freshly isolated blood of healthy donors (HDs) were cultured at a stiffness of either 0 kPa, 0.2 kPa or 50 kPa and analyzed by RNA-sequencing. Volcano plot of genes in monocytes cultured at stiffness 0 kPa, 0.2 kPa, and 50 kPa for 24 h and blue dot indicates interferon stimulated genes (ISGs).

FIG. 7C-1 and FIG. 7C-2 show that stiffness-mediated mechanosensing impacts gene expression in monocytes as noted in Example 7 Monocytes from freshly isolated blood of healthy donors (HDs) were cultured at a stiffness of either 0 kPa, 0.2 kPa or 50 kPa and analyzed by RNA-sequencing. Volcano plot of genes in monocytes cultured at stiffness 0 kPa, 0.2 kPa, and 50 kPa for 24 h and blue dot indicates fibrotic genes.

FIG. 7D-1 and FIG. 7D-2 show the levels of expression of genes of the Hippo pathway as noted in Example 7. For human, monocytes were cultured at stiffness 0 kPa for 24 h. RNA was collected and preformed RNA-sequencing. For mice, 8-10 weeks old C57BL/6 mice were either shaved or tape stripped (TAPE). After day 1, blood and skin were collected for cell sorting to isolate infiltrating monocytes (CD45⁺CD11b⁻Ly6G⁻Ly6C⁺CCR2^{low/high}) RNA were collected from the sorted cells and preformed RNA-sequencing. Expression of genes related to Hippo pathway in human and mice monocytes. Individual donors are indicated as dot.

FIG. 7E-1 and FIG. 7E-2 show that inhibiting PIEZO1 does not impact mechanosensing signaling in monocytes as noted in Example 7. Monocytes were cultured in media alone or with PIEZO1inhibitor (Dookul: 10 μM) at 0 kPA or 2 kPA for 6 h. Secretion for TNF-α and IL-6 were measured via ELISA. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 7F-1 and FIG. 7F-2 show that activating PIEZO1does not impact mechanosensing signaling in monocytes as noted in Example 7. Monocytes were cultured in media alone or with PIEZO1activator (Yodal: 20 μM) for 6 h. RNA were collected and analyzed for TNF-α and IL-1β expression. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 8A-1 and FIG. 8A-2 show that stiffness-mediated mechanosensing does not require transcription factor NFYA as noted in Example 8. Monocytes were transfected with CRISPR-Cas9 complex targeting for NFYA on using Lipofectamine™ CRISPRMAX reagent. After transfection, monocytes were cultured for 72 h, followed by 2 kPA stiffness for 6 h. RNA were collected and analyzed for NYFA, TNF-α, and IL-1β expression. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 8B-1 and FIG. 8B-2 show that stiffness-mediated mechanosensing does not induce calcium flux as noted in Example 8. Monocytes were cultured in either 0 kPA or 2 kPA stiffness for 6 h and measured the Ca²⁺ ion flux using flow cytometer. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 8C-1 and FIG. 8C-2 show that stiffness-mediated mechanosensing depends on cAMP signaling as noted in Example 8. Monocytes were cultured in media alone or with cAMP inhibitor (KH7: 40 μM) for 1 h, followed by cultured in either 0 kPA or 2 kPA stiffness for 6 h. RNA were collected and analyzed for TNF-α and IL-1β expression. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 8D-1 and FIG. 8D-2 show that stiffness-mediated mechanosensing depends on cAMP signaling as noted in Example 8. Monocytes were cultured in media alone or with cAMP activator (Forksolin: 5 μM) for 6 h. RNA were collected and analyzed for TNF-α and IL-1p expression. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 9A shows differential expression of genes in monocytes from skin versus blood after tape-stripping as noted in Example 9. 8-10 weeks old C57BL/6 mice were either shaved only or tape stripped (TAPE) to provoke inflammation. At day 1, infiltrating monocytes (CD45⁺CD11b⁻Ly6G⁻Ly6C⁺CCR2^{+(low/high)}) were FACS-sorted from the blood or inflamed skin biopsies. FACS-sorted infiltrating monocytes were analyzed by RNA-sequencing. Volcano plot showing inflammatory genes in monocytes from blood when compared between TAPE and shaved mice.

FIG. 9B shows changes in pathways in monocytes from skin versus blood after tape-stripping as noted in Example 9. 8-10 weeks old C57BL/6 mice were either shaved only or tape stripped (TAPE) to provoke inflammation. At day 1, infiltrating monocytes (CD45⁺CD11b⁻Ly6G⁻ Ly6C⁺CCR2^{+(low/high)}) were FACS-sorted from the blood or inflamed skin biopsies. FACS-sorted infiltrating monocytes were analyzed by RNA-sequencing. All DEGs were analyzed for pathways that are impacted by tape stripping in blood monocytes using QIAGEN Ingenuity Pathway Analysis (IPA). Red color indicates activation and blue color indicates inhibition.

FIG. 9C-1 and FIG. 9C-2 show that inhibiting SP1 reduces the number of CD45+ cells infiltrating the skin after tape stripping as noted in Example 9. 8-10 weeks old C57BL/6 mice were administrated intraperitoneally with either PBS or Mithramycin-A (0.5 mg/kg) for 3 consecutive days. Mice were either shaved only or tape stripped (TAPE) to provoke inflammation. After 24 h, skin biopsies were collected for flow cytometer and analyzed the infiltration of CD45⁺ cells. Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

FIG. 10A shows that mechanosensing differentially impacts the monocytes from SSc patients as noted in Example 10. Monocytes from freshly isolated blood of healthy donors (HDs) or SSc patients were cultured at a stiffness of either 0 kPa, 0.2 kPa or 50 kPa and analyzed by RNA-sequencing. RNA were collected after 24 h culture and preformed RNA-sequencing. Heatmaps of fibrotic genes that are impacted by stiffness.

FIG. 10B shows that mechanosensing modulates distinct pathways in monocytes of SSc as noted in Example 10. Monocytes from freshly isolated blood of healthy donors (HDs) or SSc patients were cultured at a stiffness of either 0 kPa, 0.2 kPa or 50 kPa and analyzed by RNA-sequencing. RNA were collected after 24 h culture and preformed RNA-sequencing. All DEGs were analyzed for pathway that are impacted in monocytes which were cultured at using QIAGEN Ingenuity Pathway Analysis. Red color indicates activation and blue color indicates inhibition.

FIG. 10C shows that mechanosensing activates distinct genes in monocytes of SSc as noted in Example 10. Monocytes from freshly isolated blood of SSc patients were cultured at a stiffness of either 0 kPa, 0.2 kPa or 50 kPa and analyzed by RNA-sequencing. RNA were collected after 24 h culture and preformed RNA-sequencing. All DEGs were analyzed for receptors that are impacted by change in stiffness using QIAGEN Ingenuity Pathway Analysis. Red color indicates activation and blue color indicates inhibition.

FIG. 10D shows that mechanosensing modulates the expression of distinct transcription factors in monocytes of SSc as noted in Example 10. Monocytes from freshly isolated blood of SSc patients were cultured at a stiffness of either 0 kPa, 0.2 kPa or 50 kPa and analyzed by RNA-sequencing. RNA were collected after 24 h culture and preformed RNA-sequencing. DEGs were analyzed for transcriptional factors that are impacted by change in stiffness using QIAGEN Ingenuity Pathway Analysis. Red color indicates activation and blue color indicates inhibition.

FIG. 11A-1, FIG. 11A-2, FIG. 11A-3, FIG. 11A-4, and FIG. 11A-5 show that skin injury by TAPE triggers inflammatory response in skin as noted in Example 11. 8-10 weeks old C57BL/6 mice were either shaved only or tape stripped (TAPE) to provoke inflammation. After day 1, skin biopsies were collected. RNA was isolated from skin biopsies and analyzed for the expression of inflammatory genes such as TNF-α, IL-1β, IP-10 and IL-6. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11B shows that skin injury by TAPE triggers infiltration of CD45+ immune cells in the inflamed skin as noted in Example 11. 8-10 weeks old C57BL/6 mice were either shaved only or tape stripped (TAPE) to provoke inflammation. After day 1, skin biopsies were collected. Infiltration of CD45⁺ cell, in skin biopsies were analyzed via flow cytometer. Individual mice are indicated; all results are represented as mean±SEM; and statistical significance was evaluated using Mann-Whitney U test or one-way ANOVA, Tukey's multiple comparisons test. *, P<0.05; **, P<0.01; ***P<0.001.

FIG. 11C shows that skin injury by TAPE triggers neutrophils infiltration in the inflamed skin as noted in Example 11. 8-10 weeks old C57BL/6 mice were either shaved only or tape stripped (TAPE) to provoke inflammation. After day 1, skin biopsies were collected. After day 1, skin biopsies were collected. Infiltration of neutrophils (CD45⁺CD11b⁺Ly6G⁺) in skin biopsies were analyzed via flow cytometer. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11D shows that skin injury by TAPE triggers macrophages infiltration in the inflamed skin as noted in Example 11. 8-10 weeks old C57BL/6 mice were either shaved only or tape stripped (TAPE) to provoke inflammation. After day 1, skin biopsies were collected. After day 1, blood and skin biopsies were collected. Infiltration of macrophages (CD45⁺CD11b⁺Ly6G⁻F480⁺) in skin biopsies were analyzed via flow cytometer. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11E shows that skin injury by TAPE triggers migration of inflammatory monocytes in the inflamed skin from blood as noted in Example 11. 8-10 weeks old C57BL/6 mice were either shaved only or tape stripped (TAPE) to provoke inflammation. After day 1, blood and skin biopsies were collected. After day 1, blood and skin biopsies were collected. Infiltration of inflammatory monocytes (CD45⁺CD11b⁺Ly6G⁻Ly6C⁺) in skin biopsies were analyzed via flow cytometer. Individual mice are indicated; all results are represented as mean±SEM; and statistical significance was evaluated using Mann-Whitney U test or one-way ANOVA, Tukey's multiple comparisons test. *P<0.05; **, P<0.01; ***, P<0.001.

FIG. 11F shows that skin injury by TAPE triggers the migration of infiltrating monocytes into inflamed skin from blood as noted in Example 11. 8-10 weeks old C57BL/6 mice were either shaved only or tape stripped (TAPE) to provoke inflammation. After day 1, blood and skin biopsies were collected. After day 1, blood and skin biopsies were collected. Infiltration of infiltrating monocytes (CD45⁺CD11b⁻Ly6G⁻Ly6C⁺CCR2^{+(low/high)}) in skin biopsies were analyzed via flow cytometer.

FIG. 11G shows that skin injury by TAPE induced the migration of infiltrating monocytes into inflamed skin as noted in Example 11. 8-10 weeks old C57BL/6 mice were either shaved only or tape stripped (TAPE) to provoke inflammation. After day 1, blood and skin biopsies were collected. After day 1, blood and skin biopsies were collected. Infiltration of infiltrating monocytes (CD45⁺CD11b⁻Ly6G⁻Ly6C⁺CCR2^{+(low/high)}) in skin biopsies were analyzed via flow cytometer. Individual mice are indicated; all results are represented as mean±SEM; and statistical significance was evaluated using Mann-Whitney U test or one-way ANOVA, Tukey's multiple comparisons test. *, P<0.05; **P<0.01; ***P<0.001.

FIG. 11H shows that change in stiffness form blood to skin activates the expression of surface marker on skin infiltrating monocytes as noted in Example 11. 8-10 weeks old C57BL/6 mice were either shaved only or tape stripped (TAPE) to provoke inflammation. After day 1, blood and skin biopsies were collected. After day 1, blood and skin biopsies were collected. CD86 expression on infiltrating monocytes (CD45⁺CD11b⁻Ly6G⁻Ly6C⁺CCR2^{+(low/high)}) in skin biopsies were analyzed via flow cytometer. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 11I-1, FIG. 11I-2, FIG. 11I-3, and FIG. 11I-4 show that change in stiffness form blood to inflamed skin induces inflammatory response in skin infiltrating monocytes as noted in Example 11. 8-10 weeks old C57BL/6 mice were either shaved only or tape stripped (TAPE) to provoke inflammation. At day 1, infiltrating monocytes were FACS-sorted from blood or inflamed skin biopsies. RNA was collected from FACS sorted cells and analyzed for the expressions of inflammatory genes such as IL-1β, CXCL2, IL-6, and TNF-α. Individual mice are indicated; all results are represented as mean±SEM; and statistical significance was evaluated using Mann-Whitney U test or one-way ANOVA, Tukey's multiple comparisons test. *, P<0.05; **, P<0.01; ***P<0.001.

FIG. 12A shows that change in stiffness form blood to skin triggers skin-infiltrating monocytes transcriptionally as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. PCA plot of RNA-seq analysis.

FIG. 12B shows that change in stiffness form blood to skin modulates inflammatory cytokines in skin-infiltrating monocytes as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Heatmaps of inflammatory cytokines in infiltrating monocytes from blood and inflamed skin biopsies.

FIG. 12C shows that skin stiffness modulates the pathways in skin-infiltrating monocytes as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. All differential regulated genes (DEGs) were analyzed for pathways using QIAGEN Ingenuity Pathway Analysis. Red colors indicate activated pathways and blue colors indicate downregulated pathways.

FIG. 12D shows that skin stiffness activates skin-infiltrating monocytes in both blood and inflamed skin as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Overlapping of DEGs in infiltrating monocytes from skin and blood in mice with shave or TAPE.

FIG. 12E shows that skin stiffness activates specific pathways in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. 1826 DEGs from FIG. 12D were analyzed using QIAGEN Ingenuity Pathway Analysis for pathway. Red color indicates activated pathways and blue color indicates downregulated pathways.

FIG. 12F shows that skin stiffness activates specific transcriptional factors in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. 1826 DEGs from FIG. 12D were analyzed using QIAGEN Ingenuity Pathway Analysis for transcription factor. Red color indicates activated transcription factor and blue color indicates downregulated transcription factor.

FIG. 12G shows that skin stiffness activates mechanosensing in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. All DEGs were analyzed for functional activation using QIAGEN Ingenuity Pathway Analysis.

FIG. 12H shows that skin stiffness activates specifically mechanosensing and cell movement in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Venn diagram of overlapping DEGs in infiltrating monocytes that are involved in either cell movement or mechanotransduction.

FIG. 121 shows that skin stiffness activates specifically cell movement in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Heatmaps of genes that are involved in cell movement.

FIG. 12J shows that skin stiffness activates specifically mechanosensing and cell movement in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Heatmaps of genes that are involved in both cell movement and mechanotransduction.

FIG. 12K shows that skin stiffness activates mechanosensing in skin-infiltrating monocytes while infiltrating into inflamed skin from blood as noted in Example 12. 8-10 weeks old C57BL/6 mice were either shaved only or tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Heatmaps of genes that are involved in mechanotransduction.

FIG. 13A shows that stiffness induces inflammatory response in human monocytes as noted in Example 13. Monocytes from freshly isolated blood of Healthy Donor (HD) were cultured at 0 kPA, 0.2 kPA or 50 kPA stiffness for 24 h. Supernatant were collected and analyzed for secretion of cytokines such as IL-1β via ELISA. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13B shows that stiffness induces inflammatory response in human monocytes as noted in Example 13. Monocytes from freshly isolated blood of Healthy Donor (HD) were cultured at 0 kPA, 0.2 kPA or 50 kPA stiffness for 24 h. Supernatant were collected and analyzed for secretion of cytokines such as TNF-α via ELISA. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13C shows that stiffness induces inflammatory response in human monocytes as noted in Example 13. Monocytes from freshly isolated blood of Healthy Donor (HD) were cultured at 0 kPA, 0.2 kPA or 50 kPA stiffness for 24 h. Supernatant were collected and analyzed for secretion of cytokines such as IL-6 via ELISA. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13D shows that stiffness-induces surface marker expression in human monocytes as noted in Example 13. Monocytes from freshly isolated blood of Healthy Donor (HD) were cultured at 0 kPA, 0.2 kPA or 50 kPA stiffness for 24 h. CD86 expressions were measured via flow cytometry. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13E shows that stiffness-induces inflammatory cytokines in human monocytes as noted in Example 13. Monocytes from freshly isolated blood of Healthy Donor (HD) were cultured at 0 kPA, 0.2 kPA or 50 kPA stiffness for 24 h. RNA was collected and RNA-sequencing was performed. Heatmaps of the inflammatory cytokines.

FIG. 13F shows that stiffness triggers fibrotic genes in human monocytes as noted in Example 13. Monocytes from freshly isolated blood of Healthy Donor (HD) were cultured at 0 kPA, 0.2 kPA or 50 kPA stiffness for 24 h. RNA was collected and RNA-sequencing was performed. Volcano plot of fibrotic genes.

FIG. 13G shows that stiffness modulates interferon stimulated genes (ISGs) in human monocytes as noted in Example 13. Monocytes from freshly isolated blood of Healthy Donor (HD) were cultured at 0 kPA, 0.2 kPA or 50 kPA stiffness for 24 h. RNA was collected and RNA-sequencing was performed. Volcano plot of ISGs.

FIG. 13H shows that stiffness exacerbates TLRs activated monocytes as noted in Example 13. Monocytes were cultured at either 0 kPA or 2 kPA stiffness for 24 h, followed by washing with fresh media. Cells were treated with either TLR4 ligand (LPS:10 ng/ml) or TLR8 ligand (ORN8L:130 μg/ml) for 6 h. Supernatant were collected and analyzed for secretion of cytokines such as IL-1β via ELISA. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 131 shows that stiffness-exacerbates TLRs activated monocytes as noted in Example 13. Monocytes from freshly isolated blood of Healthy Donor (HD) were cultured at 0 kPA, 0.2 kPA or 50 kPA stiffness for 24 h. Monocytes were cultured at either 0 kPA or 2 kPA stiffness for 24 h, followed by washing with fresh media. Cells were treated with either TLR4 ligand (LPS:10 ng/ml) or TLR8 ligand (ORN8L:130 μg/ml) for 6 h. Supernatant were collected and analyzed for secretion of cytokines such as IL-6 via ELISA. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 13J shows that stiffness exacerbates TLRs activated monocytes as noted in Example 13. Monocytes from freshly isolated blood of Healthy Donor (HD) were cultured at 0 kPA, 0.2 kPA or 50 kPA stiffness for 24 h. Monocytes were cultured at either 0 kPA or 2 kPA stiffness for 24 h, followed by washing with fresh media. Cells were treated with either TLR4 ligand (LPS:10 ng/ml) or TLR8 ligand (ORN8L:130 μg/ml) for 6 h. Supernatant were collected and analyzed for secretion of cytokines such as TNF-α via ELISA. Individual mice are indicated; all results are represented as mean±SEM; and statistical significance was evaluated using Mann-Whitney U test or one-way ANOVA, Tukey's multiple comparisons test. *, P<0.05; **, P<0.01; ***, P<0.001.

FIG. 14A shows that stiffness induces chromatin changes in human monocytes as noted in Example 14. Monocytes from freshly isolated blood of HD were cultured at either 0.2 kPA or 50 kPA stiffness for 6 h and ATAC-sequencing was performed. PCA plot of ATAC-seq analysis.

FIG. 14B shows that stiffness induces chromatin changes in human monocytes as noted in Example 14. Monocytes from freshly isolated blood of HD were cultured at either 0.2 kPA or 50 kPA stiffness for 6 h and ATAC-sequencing was performed. Enrichment heatmaps of normalized chromatin accessibility reads of ATAC-seq at 0.2 kPA and 50 kPA stiffness. The top lines represent the peak signal of each condition at 5′cut site. White and red indicate the min and max of the average signal of four replicates.

FIG. 14C shows that stiffness activates the transcription factor SP1 in human monocytes as noted in Example 14. Monocytes from freshly isolated blood of HD were cultured at either 0.2 kPA or 50 kPA stiffness for 6 h and ATAC-sequencing was performed. De novo motif analysis of differentially peaks at various stiffness using HOMER.

FIG. 14D shows that stiffness activates the transcription factor SP1 in human monocytes as noted in Example 14. Monocytes were cultured at either 0.2 kPA or 50 kPA stiffness. RNA was collected after 24 h and RNA-sequencing was performed. All DEGs were analyzed for TFs using QIAGEN Ingenuity Pathway Analysis. Red color indicates activation and blue color indicates inhibition.

FIG. 14E shows that stiffness activates the transcription factor SP1 which promotes inflammatory genes in human monocytes as noted in Example 14. Monocytes were cultured at either 0.2 kPA or 50 kPA stiffness. RNA was collected after 24 h and RNA-sequencing was performed. Transcription factor network analysis of DEGs with transcription factor SP1.

FIG. 14F-1 and FIG. 14F-2 show that stiffness activates the transcription factor SP1 in human monocytes as noted in Example 14. Monocytes were cultured at either 0.2 kPA or 50 kPA stiffness for 6 h and ATAC-sequencing was performed. JASPAR motif cut site analysis of the transcription factors NFYA and SP1 was done using HSSGenomics R scripts.

FIG. 14G-1, FIG. 14G-2, and FIG. 14G-3 show that stiffness induced the activation of SP1 which promotes inflammatory genes in human monocytes as noted in Example 14. Monocytes were transfected with CRISPR-Cas9 complex targeting for SP1 at exon-4 using Lipofectamine™ CRISPRMAX reagent. After transfection, monocytes were cultured with baricitinib (1 μM) for 24 h, followed by culturing in fresh media for 48 h, and then subjected to either 0 kPA or 2 kPA stiffness for 6 h. RNA was collected and analyzed for the expression of SP1, IL-1β, and TNF-α. Individual donors are indicated; all results are represented as mean±SEM; and statistical significance was evaluated using Mann-Whitney U test or paired t-test. *, P<0.05; **, P<0.01; ***, P<0.001.

FIG. 14H-1 and FIG. 14H-2 show that stiffness induced the activation of SP1 which promotes inflammatory genes in human monocytes as noted in Example 14. Monocytes were cultured in media alone or with mithramycin for 1 h, followed by cultured at either 0 kPA or 2 kPA stiffness for 6 h. RNA were collected and analyzed for IL-1β and TNF-α expression. Individual donors are indicated; all results are represented as mean±SEM; and statistical significance was evaluated using Mann-Whitney U test or paired t-test. *, P<0.05; **, P<0.01; ***, P<0.001.

FIG. 14I shows that stiffness promotes the binding of SP1 to the promoter of inflammatory genes in human monocytes as noted in Example 14. Monocytes were cultured at either 0.2 kPA or 50 kPA stiffness for 18 h and cells were collected for CUT&RUN assay. Track changes showing the binding of SP1 at the peak of chromatin opening observed. Representative average signal tracks showing IL-1β, TNF-α and IL-10 loci from CUT&RUN assay and ATAC-seq analysis.

FIG. 15A shows that stiffness activates SP1-regulated genes in skin-infiltrating monocytes as noted in Example 15. 8-10 weeks old C57BL/6 mice were tape stripped (TAPE) to provoke inflammation. After day 1, infiltrating monocytes (CD45⁺CD11b⁻Ly6G⁻Ly6C⁺CCR2^{+(low/high)}) were FACS-sorted from blood or inflamed skin biopsies. RNA was collected from the FACS-sorted cells and RNA-sequencing was performed. Expression of SP1-regulated genes and SP1 in log 2 CPM (count per million).

FIG. 15B-1, FIG. 15B-2, FIG. 15B-3, and FIG. 15B-4 show that SP1 inhibition in skin-infiltrating monocytes modulate skin inflammation by TAPE as noted in Example 15. 8-10 weeks old C57BL/6 mice were administrated intraperitoneally with either PBS or Mithramycin-A (0.5 mg/kg) for 3 days consecutively. Mice were either shaved only or tape stripped (TAPE) to provoke inflammation. After day 1, skin biopsies were collected and analyzed for the expression of inflammatory genes such as TNF-α, IL-1β, IP-10 and IL-6. Individual mice are indicated; all results are represented as mean±SEM; and statistical significance was evaluated using Mann-Whitney U test or one-way ANOVA, Tukey's multiple comparisons test. ns, P>0.05; *, P<0.05; **, P<0.01; ***P<0.001.

FIG. 15C shows that SP1 inhibition in skin-infiltrating monocytes reduces CD45⁺ immune cell infiltration in the inflamed skin as noted in Example 15. 8-10 weeks old C57BL/6 mice were administrated intraperitoneally with either PBS or Mithramycin-A (0.5 mg/kg) for 3 days consecutively, followed by tape stripping (TAPE). After day 1, inflamed skin biopsies were collected and analyzed for the CD45⁺ cells infiltration via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; **, P<0.01; ***, P<0.001.

FIG. 15D-1 and FIG. 15D-2 show that SP1 inhibition in skin-infiltrating monocytes reduces inflammatory genes as noted in Example 15. 8-10 weeks old C57BL/6 mice were administrated intraperitoneally with either PBS or Mithramycin-A (0.5 mg/kg) for 3 days consecutively, followed by tape stripping (TAPE). After day 1, infiltrating monocytes were FASC-sorted from blood or inflamed skin biopsies. RNA was collected and analyzed for the expressions of inflammatory genes such as IL-1β, CXCL2, IL-6, and TNF-α. Individual mice are indicated; all results are represented as mean±SEM; and statistical significance was evaluated using Mann-Whitney U test or one-way ANOVA, Tukey's multiple comparisons test. ns, P>0.05; *, P<0.05; **, P<0.01; ***, P<0.001.

FIG. 15E-1, FIG. 15E-2, FIG. 15E-3, and FIG. 15E-4 show that SP1 inhibition in skin-infiltrating monocytes modulate neutrophils infiltration as noted in Example 15. 8-10 weeks old C57BL/6 mice were administrated intraperitoneally with either PBS or Mithramycin-A (0.5 mg/kg) for 3 days consecutively, followed by tape stripping (TAPE). After day 1, blood and inflamed skin biopsies were collected and analyzed for the neutrophils infiltration via flow cytometer. Individual mice are indicated; all results are represented as mean±SEM; and statistical significance was evaluated using Mann-Whitney U test or one-way ANOVA, Tukey's multiple comparisons test. ns, P>0.05; *, P<0.05; **, P<0.01; ***, P<0.001.

FIG. 16A shows that monocytes infiltrating fibrotic skin from SSc patients have induced inflammatory genes as noted in Example 16. Single cell RNA-sequencing (sc-RNA-seq) were re-analyzed for inflammatory monocytes from published dataset (Gur et al., 2022, Cell; doi: 10.1016/j.cell.2022.03.011). U-map plot of inflammatory monocytes infiltrating skin from 56 Healthy donors and 97 SSc patients.

FIG. 16B shows that monocytes infiltrating fibrotic skin from SSc patients have induced inflammatory genes as noted in Example 16. Single cell RNA-sequencing (sc-RNA-seq) were re-analyzed for inflammatory monocytes from published dataset (Gur et al., 2022, Cell; doi: 10.1016/j.cell.2022.03.011). Expression of inflammatory cytokines in inflammatory monocytes infiltrating skin from blood in healthy donor and SSc patients.

FIG. 16C shows that stiffness differentially regulates the activation of monocytes from SSc patients as compared to healthy individual as noted in Example 16. Monocytes from freshly isolated blood of HD or SSc patients were cultured at either 0 kPA, 0.2 kPA or 50 kPA stiffness. After 24 h, RNA was collected and RNA-sequencing was performed. PCA plot of RNA-seq.

FIG. 16D shows that stiffness differentially regulates the activation of inflammatory genes in monocytes from SSc patients as compared to healthy individual as noted in Example 16. Monocytes from freshly isolated blood of HD or SSc patients were cultured at either 0 kPA, 0.2 kPA or 50 kPA stiffness. After 24 h, RNA was collected and RNA-sequencing was performed. Heatmaps of the inflammatory cytokines from monocytes of HD or SSc cultured at either 0 kPA or 0.2 kPA.

FIG. 16E shows that stiffness differentially regulates the activation of monocytes from SSc patients as compared to healthy individual as noted in Example 16. Monocytes from freshly isolated blood of HD or SSc patients were cultured at either 0 kPA, 0.2 kPA or 50 kPA stiffness. After 24 h, RNA was collected and RNA-sequencing was performed. All DEGs, in monocytes from HD or SSc cultured at 0 kPA, were analyzed for receptors using QIAGEN Ingenuity Pathway Analysis. Red color indicates activation and blue color indicates inhibition.

FIG. 16F shows that stiffness differentially regulates the activation of monocytes from SSc patients as compared to healthy individual as noted in Example 16. Monocytes from freshly isolated blood of HD or SSc patients were cultured at either 0 kPA, 0.2 kPA or 50 kPA stiffness. After 24 h, RNA was collected and RNA-sequencing was performed. Venn diagram of DEGs in monocytes from HD or SSc cultured at 0 kPA, 0.2 kPA or 50 kPA.

FIG. 16G shows that stiffness differentially regulates the activation of monocytes from SSc patients as compared to healthy individual as noted in Example 16. Monocytes from freshly isolated blood of HD or SSc patients were cultured at either 0 kPA, 0.2 kPA or 50 kPA stiffness. After 24 h, RNA was collected and RNA-sequencing was performed. Heatmaps of 606 genes from Venn diagram FIG. 16F.

FIG. 16H shows that stiffness differentially regulates the activation of genes related to resolution of inflammation in monocytes from SSc patients as compared to healthy individual as noted in Example 16. Monocytes from freshly isolated blood of HD or SSc patients were cultured at either 0 kPA, 0.2 kPA or 50 kPA stiffness. After 24 h, RNA was collected and RNA-sequencing was performed. Heatmaps of genes in cluster 4 from heatmap FIG. 16G.

FIG. 16I shows that stiffness differentially regulates the activation of genes related to resolution of inflammation in monocytes from SSc patients as compared to healthy individual as noted in Example 16. Scatter plot of DEGs in monocytes from HD or SSc cultured at either 0kPA or 50kPA.

FIG. 16J shows that stiffness differentially regulates the activation of genes related to resolution of inflammation (wound healing) in monocytes from SSc patients as compared to healthy individual as noted in Example 16. Monocytes were transfected with CRISPRCas9 complex targeting for VAV3 using Lipofectamine™ CRISPRMAX reagent. After transfection, monocytes were cultured with baricitinib (1 μM) for 24 h, followed by culturing in fresh media for 48 h, and then subjected to either 0 kPA or 0.2 kPA stiffness for 6 h. RNA was collected and analyzed for the expression of VAV3. Individual donors are indicated; all results are represented as mean±SEM; and statistical significance was evaluated using paired t-test. *, P<0.05; **, P<0.01; ***, P<0.001.

FIG. 16K-1 and FIG. 16K-2 show that stiffness differentially regulates the activation of genes related to resolution of inflammation (wound healing) in monocytes from SSc patients as compared to healthy individual as noted in Example 16. Monocytes were transfected with CRISPRCas9 complex targeting for VAV3 using Lipofectamine™ CRISPRMAX reagent. After transfection, monocytes were cultured with baricitinib (1 μM) for 24 h, followed by culturing in fresh media for 48 h, and then subjected to either 0 kPA or 0.2 kPA stiffness for 6 h. RNA was collected and analyzed for the expression of IL-1β and CXCL8. Individual donors are indicated; all results are represented as mean±SEM; and statistical significance was evaluated using paired t-test. *P<0.05; **, P<0.01; ***, P<0.001.

FIG. 17A shows that skin injury by TAPE activates blood monocytes monocytes as noted in Example 17. 8-10 weeks old C57BL/6 mice were either shaved only or tape stripped (TAPE) to provoke inflammation as noted in Example 11. RNA were isolated from skin biopsies at day 1, 3, and 5, and analyzed for the expression of inflammatory genes such as TNF-α. Individual donors are indicated; all results are represented as mean±SEM.

FIG. 17B shows that skin injury by TAPE activates blood monocytes as noted in Example 11. 8-10 weeks old C57BL/6 mice were either shaved only or tape stripped (TAPE) to provoke inflammation. RNA were isolated from skin biopsies at day 1, 3, and 5, and analyzed for the expression of inflammatory genes such as IL-1b. Individual donors are indicated; all results are represented as mean±SEM.

FIG. 17C shows that skin injury by TAPE activates blood monocytes by modulating pathways as noted in Example 12. 8-10 weeks old C57BL/6 mice were either shaved or tape stripping (TAPE). After day 1, blood was collected for FACS-sorting to isolate infiltrating monocytes (CD45⁺CD11b⁻Ly6G⁻Ly6C⁺CCR2^{+(low/high)}) RNA were collected from the sorted cells and preformed RNA-sequencing. All DEGs were analyzed for pathways that are impacted by TAPE using QIAGEN Ingenuity Pathway Analysis. Red colors indicate activated pathways and blue colors indicate inhibited pathways.

FIG. 17D shows that skin injury by TAPE activates blood monocytes by modulating pathways as noted in Example 12. 8-10 weeks old C57BL/6 mice were either shaved or tape stripping (TAPE). After day 1, blood was collected for FACS-sorting to isolate infiltrating monocytes (CD45⁺CD11b⁻Ly6G⁻Ly6C⁺CCR2^{+(low/high)}) RNA were collected from the sorted cells and preformed RNA-sequencing. Volcano plot of gene related to activated pathways and inflammatory genes in monocytes from blood compared in between TAPE and Shave.

FIG. 17E shows that skin injury by TAPE activates monocytes in blood as noted in Example 12. Venn diagram of overlapping DEGs in infiltrating monocytes that are involved between inflammatory response (from activated cytokine storm signaling pathways) and mechanotransduction with cell movement.

FIG. 17F shows that skin injury by TAPE activates monocytes in blood as noted in Examples 12 and 16. Venn diagram of overlapping DEGs in infiltrating monocytes that are involved between inflammatory response (from activated cytokine storm signaling pathways) and mechanotransduction with cell movement.

FIG. 18A-1 and FIG. 18A-2 show that the Hippo pathway is not involved in stiffness-mediated mechanosensing in monocytes as noted in Example 14. Expression of genes related to Hippo pathway in human and mice monocytes.

FIG. 18B shows that monocytes infiltrating fibrotic skin from SSc patients have induced inflammatory genes as noted in Example 14. Single cell RNA-sequencing (sc-RNA-seq) were re-analyzed for infiltrating monocytes from published dataset (Gur et al., 2022, Cell; doi: 10.1016/j.cell.2022.03.011). Expression of genes related to the hippo pathway in inflammatory monocytes from blood and skin of both healthy donors and SSc patients.

FIG. 18C shows that the PIEZO1signaling is not involved in stiffness-mediated mechanosensing in human monocytes as noted in Example 14. Monocytes were cultured in media alone or with PIEZO1inhibitor (Dookul:10 μM) at 0 kPA or 2 kPA for 6 h. Supernatant were collected and analyzed for secretion for TNF-α and IL-6 secretion via ELISA. Individual donors are indicated; all results are represented as mean±SEM; and statistical significance was evaluated using Mann-Whitney U test. ns, P>0.05; *, P<0.05; **, P<0.01; ***, P<0.001.

FIG. 18D-1 and FIG. 18D-2 show that the PIEZO1signaling is not involved in stiffness-mediated mechanosensing in human monocytes as noted in Example 14. Monocytes were cultured in media alone or with PIEZO1activator (Yodal: 20 μM) for 6 h. RNA were collected and analyzed for TNF-α and IL-1β expression. Individual donors are indicated; all results are represented as mean±SEM; and statistical significance was evaluated using Mann-Whitney U test. ns, P>0.05; *, P<0.05; **, P<0.01; ***, P<0.001.

FIG. 18E shows that stiffness activates SP1transcription factor that induces inflammatory genes in human monocytes as noted in Example 15. Monocytes were cultured at 0 kPA, 0.2 kPA and 50 kPA stiffness. After 24 h, RNA were collected, and RNA-sequencing was performed. Expression of SP1-regulated genes and SP1 in log 2 CPM (count per million).

FIG. 19A-1, FIG. 19A-2, and FIG. 19A-3 shows that monocytes infiltrating fibrotic skin from SSc patients have induced inflammatory genes as noted in Example 16. Single cell RNA-sequencing (sc-RNA-seq) were re-analyzed for infiltrating monocytes from published dataset (Gur et al., 2022, Cell; doi: 10.1016/j.cell.2022.03.011). Expression of inflammatory genes such as IL-1b, CXCL2, and CXCL8 in inflammatory monocytes from skin of SSc patients.

FIG. 19B shows that stiffness differentially regulates the fibrotic genes in monocytes from SSc patients as compared to healthy individual as noted in Example 16. Monocytes from freshly isolated blood of HD or SSc patients were cultured in stiffness at 0 kPA, 0.2 kPA and 50 kPA. RNA were collected after 24 h culture and RNA-sequencing was performed. Heatmaps of fibrotic genes that are impacted by stiffness.

FIG. 19C shows that stiffness activates transcriptional factor SP1 in monocytes from SSc patients as noted in Example 16. Monocytes from freshly isolated blood of SSc patients were cultured in stiffness at 0 kPA, 0.2 kPA and 50 kPA. RNA were collected after 24 h culture and RNA-sequencing was performed. All DEGs were analyzed for TFs in monocytes from SSc patients at 0.2 kPA and 50 kPA using QIAGEN Ingenuity Pathway Analysis. Red colors indicate activation and blue colors indicate inhibition.

FIG. 19D shows that stiffness activates different pathways in monocytes from SSc patients as noted in Example 16. Monocytes from freshly isolated blood of SSc patients were cultured in stiffness at 0 kPA, 0.2 kPA and 50 kPA. RNA were collected after 24 h culture and RNA-sequencing was performed. All DEGs were analyzed for pathway in HD vs SSc at 0 kPA using QIAGEN Ingenuity Pathway Analysis pathways that are impacted using QIAGEN Ingenuity Pathway Analysis. Red colors indicate activated pathways and blue colors indicate inhibited pathways.

FIG. 20A shows that skin injury by TAPE induced the expression of genes related to resolution of inflammation in infiltrating monocytes. 8-10 weeks old C57BL/6 mice were either shaved or tape stripping (TAPE). After day 1, blood and skin was collected for FACS sorting to isolate infiltrating monocytes (CD45⁺CD11b⁻Ly6G⁻Ly6C⁺CCR2^{+(low/high)}) RNA were collected from the FACS-sorted cells and RNA-sequencing was performed. Gene Expression of VAV3. Individual mice are indicated; all results are represented as mean±SEM; and statistical significance was evaluated using Mann-Whitney U test. ns, P>0.05; *, P<0.05; **, P<0.01; ***, P<0.001.

FIG. 20B shows that skin injury by TAPE induced the expression of genes related to resolution of inflammation in infiltrating monocytes. 8-10 weeks old C57BL/6 mice were either shaved or tape stripping (TAPE). After day 1, blood and skin was collected for FACS sorting to isolate infiltrating monocytes (CD45⁺CD11b⁻Ly6G⁻Ly6C^{+CCR2+(low/high)}) RNA were collected from the FACS-sorted cells and RNA-sequencing was performed. Gene Expression of GPR84. Individual mice are indicated; all results are represented as mean±SEM; and statistical significance was evaluated using Mann-Whitney U test. ns, P>0.05; *, P<0.05; **, P<0.01; ***, P<0.001.

FIG. 20C shows that skin injury by TAPE induced the expression of genes related to resolution of inflammation in infiltrating monocytes. 8-10 weeks old C57BL/6 mice were either shaved or tape stripping (TAPE). After day 1, blood and skin was collected for FACS sorting to isolate infiltrating monocytes (CD45⁺CD11b⁻Ly6G⁻Ly6C⁺CCR2^{+(low/high)}) RNA were collected from the FACS-sorted cells and RNA-sequencing was performed. Gene Expression of GJB2. Individual mice are indicated; all results are represented as mean±SEM; and statistical significance was evaluated using Mann-Whitney U test. ns, P>0.05; *, P<0.05; **, P<0.01; ***, P<0.001.

### DETAILED DESCRIPTION

Monocytes are pivotal in inflammation and autoimmunity, migrating from the bloodstream to tissues like the skin. However, what governs their behavior in the skin has remained unclear. The studies disclosed herein reveal that skin-infiltrating monocytes sense skin stiffness initiating mechanosensing and produce inflammatory cytokines like IL-1β, IL-6, and TNF-α via activation of the transcription factor SP1. Upon disruption of SP1 activation in vivo, a reduction of skin inflammation was observed, marked by a decrease in the expression of inflammatory genes like IL-1β and CXCL2, which are typically produced by skin-infiltrating monocytes. In patients with SSc, inflammatory monocytes infiltrating the skin exhibited a chronically inflammatory response as compared to their counterpart in the blood, while mechanosensing from fibrotic skin stiffness selectively suppressed resolution mechanisms such as anti-inflammatory, wound healing, and fibrotic responses, maintaining a chronic activation status. During inflammation or injury, monocytes can infiltrate tissues and in response to the sensing of PAMPs or DAMPs, these cells will induce an inflammatory response^{13,45-47} What was unclear is the role played by the tissue environment and how this controls monocyte response. Hence, the data disclosed herein unveil a critical, previously undescribed mechanism by which monocytes are regulated by stiffness-induced mechanosensing which can lead to chronic inflammatory responses in the context of autoimmune and/or fibrotic diseases.

Although mechanosensing has been shown to impact macrophages in mice, the mechanism is unclear. It is well defined that inflammatory cytokines such as TNF-α, IL-1β, and IL-6 can be induced in monocytes & macrophages via the engagement of NF-κB and STAT6⁴¹⁻⁴³. However, at least in mice, it was shown that stiffness does not activate the phosphorylation of NF-κB and STAT6 in BMDM⁷¹, suggesting that stiffness-mediated mechanosensing can activate these cells via previously undescribed pathways. Multiple signaling pathways have been identified in various cellular contexts, including in neurons where PIEZO1/2 signaling can be induced by stimuli like pain, touch, or impact^{17,18}. Furthermore, PIEZO1 signaling was shown to be induced by cyclic pressure in BMDM through endothelin secretion¹⁸. However, the data disclosed herein indicate that PIEZO1 signaling is not implicated in the mechanosensing of human monocytes. Similarly, it was observed that key components of other known pathways involved in mechanosensing such as the hippo pathway³⁵ are not involved. It was observed that YAP1, which plays a role in mechanotransduction mediated inflammatory response⁷² in BMDM and importantly the TEADs, are not readily expressed in human monocytes. These discrepancies may stem from the variation of sources of mechanosensing or differences in immune cell types. Indeed, both Solis et al. and Meli et al. differentiated mouse BMDM into macrophages in a 7-days culture, which may modify the transcriptional and epigenetic profiles of these cells^{73,74}, whereas freshly purified human monocytes from blood were used in the studies described herein. Considering these observations, other TFs were explored that could be induced by mechanosensing in human monocytes and potentially other immune cells.

The genomic analysis described herein revealed that stiffness activated mechanosensing via the TF SP1, which subsequently governed the expression of inflammatory cytokines such as TNF-α, IL-10, and IL-1β. Previous studies showed that shear stress in bovine aortic endothelial cells induces tissue factor mRNA expression through SP1 phosphorylation, rather than enhancing SP1 binding to the promoter⁷⁵. However, in the CUT&RUN analysis disclosed herein, notable changes in SP1 binding to the promoters of IL-1β, TNF-α, and IL-10 in response to stiffness change were observed, suggesting that SP1 activates IL-1β, TNF-α, and IL-10 via binding to their promoter. It is also worth noting that SP1 has been associated with both the induction and suppression of gene expression. Specifically, SP1 plays a pivotal role in inducing IL-10 expression in the RAW264.7 macrophage cell line when exposed to LPS⁵¹. The ATAC-seq analysis disclosed herein indicated chromatin opening in the IL-10 promoter region induced by mechanosensing, while CUT&RUN analysis demonstrated mechanosensing causing the loss of SP1 binding in that region. This suggests that SP1 might recruit repressive elements to the opened chromatin in response to mechanosensing. These findings align with the RNA-seq data disclosed herein, where mechanosensing induced the expression of IL-1β and TNF-α while suppressing the expression of IL-10.

The link between monocytes and macrophages and skin inflammation, leading to either tissue repair or fibrosis, has been extensively studied in health but also in the context of diseases such as SSc^{6-8,11,12,24,25}. In SSc patients, the sensing of nucleic acids by TLR7, TLR8 and TLR9 plays a crucial role in initiating inflammatory responses^{12,13,76}. The transcriptomic analysis of blood monocytes from patients with SSc disclosed herein, has revealed that the inflammatory signatures observed in SSc monocytes resulted not only from TLRs activation but also from the partial activation of mechanosensing, potentially involving the TREM1 receptor. It is known that the extracellular cold-inducible RNA-binding protein (eCIRP) acts as a biologically active endogenous ligand, triggering TREM1 in conditions like sepsis⁷⁷. Additionally, previous work highlighted a potential role for circulating anti-TREM1 antibodies to activate the TREM1 receptor in vivo⁷⁸.

Hence, it can be postulated that in patients with SSc, TREM-1 agonists, such as eCIRP or anti-TREM1 antibodies, may be present, fueling the activation of monocytes alongside TLR ligands. These various epigenetic and transcriptional changes induced by high stiffness on blood monocytes of SSc patients cannot precisely be mirrored by the blood monocytes of a mouse model for autoimmune diseases. This will require further investigations. It also remains unclear why monocytes that infiltrate fibrotic skin maintain a chronic activation status, fostering inflammation and fibrosis.

By studying monocytes from the blood of patients with SSc, a dysregulation in pathways associated with the resolution of the inflammation was observed. Specifically, the expression of VAV3 and GRP84, which are associated with wound healing and anti-fibrotic responses^{64,66}, are increased in cells from patients with SSc. However, upon increased stiffness, these are reduced in cells from patients, while they increase in cells from healthy donors and their downregulation via CRISPR/Cas9 leads to increased inflammatory response. Why such pathways are impacted in circulating monocytes of patients is unclear and will require further investigation, but it seems that the lack of resolution mechanism to suppress proinflammatory response might be responsible for chronic inflammatory response in SSc patients.

As disclosed herein, to test the role of stiffness on monocyte response and specifically of how SP1 regulate this response, a well-defined mild skin injury model by performing tape stripping was used. This model presents numerous advantages. First, tape stripping provokes the recruitment of multiple cell types to the skin, including pDCs or neutrophils, but also of monocytes. Second, this model associates multiple factors that leads to inflammation, including the presence of DAMPS/PAMPs but also of stiffness which is about 200-fold higher than in the blood. Hence, following tape stripping, monocytes were quickly exposed to increased stiffness. The monocytes were FACS-sorted from the skin at different time points which allowed for conducting deep sequencing of skin-infiltrating cells and comparing with blood monocytes, which helped to identify key pathways induced in the cells. This model was used to show that SP1 inhibition significantly regulated the expression of IL-1β and CXCL2, while it had less impact on TNF-α, a finding that fits with the human ex-vivo experiment and suggested that genes such as IL-1β may be more dependent on mechanosensing than by TLR signaling in these infiltrating cells. Additionally, the re-analysis of sc-RNA-seq from samples from the skin of patients with SSc revealed that inflammatory monocytes are the primary producers of IL-1βp, CXCL2 and CXCL8 in the skin of SSc patients. Overall, the findings disclosed herein support the concept that interfering with pathways induced by mechanosensing in skin-infiltrating monocytes may represent a novel strategy to interfere with the chronic inflammation and fibrosis observed in patients with SSc and other autoinflammatory diseases.

In some embodiments, the present disclosure provides, inter alia, a method of inhibiting mechanosensing in monocytes, said method comprising inhibition of the SP1-mediated mechanosensing pathway.

In some embodiments, the inhibition of the SP1-mediated mechanosensing pathway is via inhibition of SP1 transcription factor.

In some embodiments, the inhibition of SP1 transcription factor is by exposure to an SP1 transcription factor inhibitor.

In some embodiments, the SP1 transcription factor inhibitor is mithramycin-A.

In some embodiments, the SP1 transcription factor inhibitor is Withaferin A.

In some embodiments, the SP1 transcription factor inhibitor is an analog of mithramycin. In some embodiments, the SP1 transcription factor inhibitor is EC-8042.

In some embodiments, the inhibition of the SP1-mediated mechanosensing pathway is via antagonism of the TREM1 receptor.

In some embodiments, the antagonism of the TREM1 receptor is by exposure to a TREM1 receptor antagonist.

In some embodiments, the TREM1 receptor antagonist is nangibotide. In some embodiments, the TREM1 receptor antagonist is VJDT. In some embodiments, the TREM1 receptor antagonist is LP17 inhibitory peptide.

In some embodiments, the inhibition of the SP1-mediated mechanosensing pathway is via antagonism of the GPR30 receptor.

In some embodiments, the antagonism of the GPR30 receptor is by exposure to a GPR30 receptor antagonist.

In some embodiments, the GPR30 receptor antagonist is G15.

In some embodiments, the GPR30 receptor antagonist is fulvestrant.

In some embodiments, the GPR30 receptor antagonist is fulvestrant-d3.

In some embodiments, the GPR30 receptor antagonist is G36.

In some embodiments, the monocytes are in skin tissue.

In some embodiments, the monocytes are human monocytes.

In some embodiments, inhibiting mechanosensing reduces inflammatory cytokines TNF-α, IL-1β, and/or IL-6.

In some embodiments, the inflammatory cytokines are reduced in infiltrating monocytes in inflamed skin.

In some embodiments, the present disclosure provides, inter alia, a method of treating a disease or disorder associated with inhibiting mechanosensing in monocytes, said method comprising inhibition of the SP1-mediated mechanosensing pathway.

In some embodiments, the inhibition of the SP1-mediated mechanosensing pathway is via inhibition of SP1 transcription factor.

In some embodiments, the inhibition of SP1 transcription factor is by administration of an SP1 transcription factor inhibitor.

In some embodiments, the SP1 transcription factor inhibitor is mithramycin-A.

In some embodiments, the SP1 transcription factor inhibitor is Withaferin A.

In some embodiments, the SP1 transcription factor inhibitor is an analog of mithramycin. In some embodiments, the SP1 transcription factor inhibitor is EC-8042.

In some embodiments, the inhibition of the SP1-mediated mechanosensing pathway is via antagonism of the TREM1 receptor.

In some embodiments, the antagonism of the TREM1 receptor is by administration of a TREM1 receptor antagonist.

In some embodiments, the TREM1 receptor antagonist is nangibotide. In some embodiments, the TREM1 receptor antagonist is VJDT. In some embodiments, the TREM1 receptor antagonist is LP17 inhibitory peptide.

In some embodiments, the inhibition of the SP1-mediated mechanosensing pathway is via antagonism of the GPR30 receptor.

In some embodiments, the antagonism of the GPR30 receptor is by exposure to a GPR30 receptor antagonist.

In some embodiments, the GPR30 receptor antagonist is G15.

In some embodiments, the GPR30 receptor antagonist is fulvestrant.

In some embodiments, the GPR30 receptor antagonist is fulvestrant-d3.

In some embodiments, the GPR30 receptor antagonist is G36.

In some embodiments, the disease or disorder is an autoimmune disorder or a fibrotic disorder.

In some embodiments, the disease or disorder is an autoimmune disorder.

In some embodiments, the disease or disorder is a fibrotic disorder.

In some embodiments, the disease or disorder is a skin disease or disorder.

In some embodiments, the disease or disorder is fibrosis.

In some embodiments, the disease or disorder is systemic lupus erythematosus (SLE).

In some embodiments, the skin disease or disorder is systemic sclerosis.

In some embodiments, the skin disease or disorder is psoriasis.

In some embodiments, the skin disease or disorder is scleroderma.

In some embodiments, the skin disease or disorder is cutaneous lupus.

In some embodiments, the cutaneous lupus is acute cutaneous lupus (ACLE).

In some embodiments, the cutaneous lupus is chronic cutaneous lupus (discoid lupus).

In some embodiments, the cutaneous lupus is cutaneous lupus erythematosus (CLE).

In some embodiments, the skin disease or disorder is dermatomyositis.

In some embodiments, the skin disease or disorder is Behcet's Disease.

In some embodiments, the skin disease or disorder is ocular cicatricial pemphigoid (OCP).

In some embodiments, the skin disease or disorder is pemphigus.

In some embodiments, the skin disease or disorder is epidermolysis bullosa.

In some embodiments, the skin disease or disorder is epidermolysis bullosa acquisita (EBA).

In some embodiments, the skin disease or disorder is bullous pemphigoid.

In some embodiments, the skin disease or disorder is lichen planus.

In some embodiments, the skin disease or disorder is lichen sclerosus.

In some embodiments, the skin disease or disorder is a wound.

It is further appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination. Thus, it is contemplated as features described as embodiments of the compounds of the disclosure can be combined in any suitable combination.

As used herein, the term “administration” typically refers to the administration of a composition to a subject or system. Those of ordinary skill in the art will be aware of a variety of routes that may, in appropriate circumstances, be utilized for administration to a subject, for example a human subject. In some embodiments, administration may involve dosing that is intermittent (e.g., a plurality of doses separated in time) and/or periodic (e.g., individual doses separated by a common period of time) dosing.

GPER1 (G protein-coupled estrogen receptor 1), is a protein encoded by the GPER gene, and is also known as GPR30 (G protein-coupled receptor 30). The GPR30 receptor antagonists G15, fulvestrant, fulvestrant-d3, G36 are commercially available, e.g., from MedChemExpress LLC, Monmouth Junction, NJ, USA (www.medchemexpress.com).

Mithramycin A, also known as mithramycin, Mithracin, and plicamycin, has been shown to inhibit SP1 transcription factor. Analogs of mithramycin A such as EC-8042 have also been shown to inhibit SP1 transcription factor (Tornin, et al., Oncotarget. 2016; 7:30935-30950. https://doi.org/10.18632/oncotarget.8817).

The terms “subject,” “individual,” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the methods are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

As disclosed in the Examples, matrigel was used with different stiffness and blocks its effect with inhibitors, activators, CRISPR-Cas9 system, metabolites and cytokines. The following observations were made in the Examples:
1. Tissue microenvironments induces inflammatory cytokines such as TNF-α, IL-1β, IL-6, CXCL2 in infiltrating monocytes in inflamed skin.
2. Increasing stiffness or physical pressure induces inflammatory cytokines production such as TNF-α, IL-1β, and IL-6 in human monocytes.
3. Blocking TREM1 receptor inhibits stiffness-mediated TNF-αinduction.
4. Blocking GPER1 (GPR30) receptor inhibits stiffness-mediated IL-1β induction.
5. Blocking actin polymerization have on effect on stiffness-mediated activation of monocytes.
6. Hippo pathway components such as LATS1/2 and TAZ, only express in both human and mice monocytes, but not other components such as YAP1 and TEADs1/2/3/4.
7. Increasing stiffness or physical pressure activates transcriptional factor-SP1 in human monocytes.
8. Inhibition of SP1 by mithramycin-A inhibit stiffness-mediated activation of inflammatory cytokines such as TNF-α, IL-1β, and IL-6 in human monocytes
9. SP1 binds to promoter of inflammatory genes such as TNF-α, IL-1β, and IL-10 in human monocytes to induces its expression.
10. cAMP is involved in stiffness-mediated activation of inflammatory cytokines such as TNF-α, IL-1β, and IL-6 in human monocytes.
11. Inhibition of SP1 by mithramycin-A in Tape stripping mice reduces inhibit inflammation by reducing inflammatory cytokines such as TNF-α, IL-1β, and IL-6 in infiltrating monocytes.
12. Monocytes from SSc patient express high level of inflammatory cytokines such as TNF-α, IL-1β, CXCL8, CCL4 and IL-6 in skin compared to the bloodstream.
13. Monocytes from blood of SSc patient are activated compared to Healthy donors in partial resemblance of mechanosensing via activation of TREM1 receptors.
14. Mechanosensing reduced anti-inflammatory response, wound healing and anti-fibrotic response in monocytes from SSc patient at 50 kPA (fibrotic skin condition) compared to 0 kPA (blood) and promotes chronic inflammation.

### Combination Therapies

Compounds of the present disclosure, e.g., SP1 transcription factor inhibitors such as mithramycin-A, or pharmaceutically acceptable salts thereof, can be used in combination with one or more additional therapeutic agents for the treatment of diseases, such as fibrosis, systemic lupus erythematosus (SLE), systemic sclerosis, psoriasis, scleroderma, cutaneous lupus, acute cutaneous lupus (ACLE), chronic cutaneous lupus (discoid lupus), cutaneous lupus erythematosus (CLE), dermatomyositis, Behcet's Disease, ocular cicatricial pemphigoid (OCP), pemphigus, epidermolysis bullosa, epidermolysis bullosa acquisita (EBA), bullous pemphigoid, lichen planus, or lichen sclerosus.

When more than one pharmaceutical agent is administered to a patient, they can be administered simultaneously, separately, sequentially, or in combination (e.g., for more than two agents).

### Formulation, Dosage Forms and Administration

When employed as pharmaceuticals, the compounds of the present disclosure, e.g., SP1 transcription factor inhibitors such as mithramycin-A, can be administered in the form of pharmaceutical compositions. Thus, the present disclosure provides a composition comprising a compound described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions for use in the methods herein, which contain, as the active ingredient, a compound of the present disclosure or a pharmaceutically acceptable salt thereof, in combination with one or more pharmaceutically acceptable carriers or excipients. In some embodiments, the composition is suitable for topical administration. In making the compositions of the disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 50 mg to about 400 mg, of the active ingredient. In some embodiments, each dosage contains about 50 mg of the active ingredient. In some embodiments, each dosage contains about 100 mg of the active ingredient. In some embodiments, each dosage contains about 200 mg of the active ingredient. In some embodiments, each dosage contains about 300 mg of the active ingredient. In some embodiments, each dosage contains about 400 mg of the active ingredient.

In some embodiments, the compound is administered to the patient at a daily dose in the range of about 50 mg/day to about 400 mg/day. In some embodiments, the compound is administered to the patient at a daily dose in the range of about 50 mg/day to about 300 mg/day, about 50 mg/day to about 300 mg/day, about 50 mg/day to about 200 mg/day, about 50 mg/day to about 100 mg/day, about 50 mg/day to about 75 mg/day, about 50 mg/day to about 60 mg/day, about 300 mg/day to about 400 mg/day, about 200 mg/day to about 400 mg/day, or about 100 mg/day to about 300 mg/day.

In some embodiments, the compound is administered to the patient at a daily dose of about 50 mg/day. In some embodiments, the compound is administered to the patient at a daily dose of about 100 mg/day. In some embodiments, the compound is administered to the patient at a daily dose of about 200 mg/day. In some embodiments, the compound is administered to the patient at a daily dose of about 300 mg/day. In some embodiments, the compound is administered to the patient at a daily dose of about 400 mg/day. In some embodiments, the compound is administered to the patient at a daily dose of about 500 mg/day. In some embodiments, the compound is administered to the patient at a daily dose of about 750 mg/day. In some embodiments, the compound is administered to the patient at a daily dose of about 1000 mg/day. In some embodiments, the compound is administered to the patient at a daily dose of about 10 mg/day. In some embodiments, the compound is administered to the patient at a daily dose of about 1 mg/day.

In some embodiments, the daily dose is in the range of about 1 mg/day to about 1000 mg/day, about 10 mg/day to about 750 mg/day, about 10 mg/day to about 500 mg/day, about 10 mg/day to about 400 mg/day, about 10 mg/day to about 300 mg/day, about 10 mg/day to about 200 mg/day, about 10 mg/day to about 100 mg/day, about 10 mg/day to about 50 mg/day, about 50 mg/day to about 500 mg/day, about 50 mg/day to about 400 mg/day, about 50 mg/day to about 300 mg/day, about 50 mg/day to about 200 mg/day, or about 50 mg/day to about 100 mg/day. In some aspects, the method includes administering to the patient a single dose of the composition. In some aspects, the method includes administering to the patient multiple doses of the composition. In some aspects, the method includes administering to the patient from 1 to 4 doses of the composition per day.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 Dg/kg to about 1 g/kg of body weight per day. In some embodiments, the dose range is from about 0.01 mg/kg to about 100 mg/kg of body weight per day. In some embodiments, the dose range is from about 0.02 mg/kg to about 20 mg/kg, about 0.05 mg/kg to about 10 mg/kg, 0.1 mg/kg to about 10 mg/kg, 0.2 mg/kg to about 8 mg/kg, 0.5 mg/kg to about 5 mg/kg, 1 mg/kg to about 5 mg/kg, or 2 mg/kg to about 3 mg/kg of body weight per day. In some embodiments, the dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg of body weight per day.

The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt. % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### REFERENCES

1. Meizlish, M. L., Franklin, R. A., Zhou, X., and Medzhitov, R. (2021). Tissue Homeostasis and Inflammation. Annu Rev Immunol 39, 557-581. 10.1146/annurev-immunol-061020-053734.
2. Hamidzadeh, K., Christensen, S. M., Dalby, E., Chandrasekaran, P., and Mosser, D. M. (2017). Macrophages and the Recovery from Acute and Chronic Inflammation. Annu Rev Physiol 79, 567-592. 10.1146/annurev-physiol-022516-034348.
3. Shook, B. A., Wasko, R. R., Rivera-Gonzalez, G. C., Salazar-Gatzimas, E., Lopez-Giraldez, F., Dash, B. C., Munoz-Rojas, A. R., Aultman, K. D., Zwick, R. K., Lei, V., et al. (2018). Myofibroblast proliferation and heterogeneity are supported by macrophages during skin repair. Science 362. 10.1126/science.aar2971.
4. Gieseck, R. L., 3rd, Wilson, M. S., and Wynn, T. A. (2018). Type 2 immunity in tissue repair and fibrosis. Nat Rev Immunol 18, 62-76. 10.1038/nri.2017.90.
5. Vannella, K. M., and Wynn, T. A. (2017). Mechanisms of Organ Injury and Repair by Macrophages. Annu Rev Physiol 79, 593-617. 10.1146/annurev-physiol-022516-034356.
6. Wynn, T. A., and Vannella, K. M. (2016). Macrophages in Tissue Repair, Regeneration, and Fibrosis. Immunity 44, 450-462. 10.1016/j.immuni.2016.02.015.
7. Shi, C., Velazquez, P., Hohl, T. M., Leiner, I., Dustin, M. L., and Pamer, E. G. (2010). Monocyte trafficking to hepatic sites of bacterial infection is chemokine independent and directed by focal intercellular adhesion molecule-1 expression. J Immunol 184, 6266-6274. 10.4049/jimmunol.0904160.
8. Shi, C., and Pamer, E. G. (2011). Monocyte recruitment during infection and inflammation. Nat Rev Immunol 11, 762-774. 10.1038/nri3070.
9. Pasparakis, M., Haase, I., and Nestle, F. O. (2014). Mechanisms regulating skin immunity and inflammation. Nat Rev Immunol 14, 289-301. 10.1038/nri3646.
10. Kratofil, R. M., Kubes, P., and Deniset, J. F. (2017). Monocyte Conversion During Inflammation and Injury. Arterioscler Thromb Vasc Biol 37, 35-42. 10.1161/ATVBAHA. 116.308198.
11. Guilliams, M., Mildner, A., and Yona, S. (2018). Developmental and Functional Heterogeneity of Monocytes. Immunity 49, 595-613. 10.1016/j.immuni.2018.10.005.
12. Narasimhan, P. B., Marcovecchio, P., Hamers, A. A. J., and Hedrick, C. C. (2019). Nonclassical Monocytes in Health and Disease. Annu Rev Immunol 37, 439-456. 10.1146/annurev-immunol-042617-053119.
13. Barrat, F. J., Elkon, K. B., and Fitzgerald, K. A. (2016). Importance of Nucleic Acid Recognition in Inflammation and Autoimmunity. Annu Rev Med 67, 323-336. 10.1146/annurev-med-052814-023338.
14. Martino, F., Perestrelo, A. R., Vinarsky, V., Pagliari, S., and Forte, G. (2018). Cellular Mechanotransduction: From Tension to Function. Front Physiol 9, 824. 10.3389/fphys.2018.00824.
15. Du, H., Bartleson, J. M., Butenko, S., Alonso, V., Liu, W. F., Winer, D. A., and Butte, M. J. (2023). Tuning immunity through tissue mechanotransduction. Nat Rev Immunol 23, 174-188. 10.1038/s41577-022-00761-w.
16. Wagh, K., Ishikawa, M., Garcia, D. A., Stavreva, D. A., Upadhyaya, A., and Hager, G. L. (2021). Mechanical Regulation of Transcription: Recent Advances. Trends Cell Biol 31, 457-472. 10.1016/j.tcb.2021.02.008.
17. Coste, B., Mathur, J., Schmidt, M., Earley, T. J., Ranade, S., Petrus, M. J., Dubin, A. E., and Patapoutian, A. (2010). Piezo1 and Piezo2 are essential components of distinct mechanically activated cation channels. Science 330, 55-60. 10.1126/science.1193270.
18. Solis, A. G., Bielecki, P., Steach, H. R., Sharma, L., Harman, C. C. D., Yun, S., de Zoete, M. R., Warnock, J. N., To, S. D. F., York, A. G., et al. (2019). Mechanosensation of cyclical force by PIEZO1 is essential for innate immunity. Nature 573, 69-74. 10.1038/s41586-019-1485-8.
19. Jain, N., Moeller, J., and Vogel, V. (2019). Mechanobiology of Macrophages: How Physical Factors Coregulate Macrophage Plasticity and Phagocytosis. Annu Rev Biomed Eng 21, 267-297. 10.1146/annurev-bioeng-062117-121224.
20. Handorf, A. M., Zhou, Y., Halanski, M. A., and Li, W. J. (2015). Tissue stiffness dictates development, homeostasis, and disease progression. Organogenesis 11, 1-15. 10.1080/15476278.2015.1019687.
21. Lagares, D., Santos, A., Grasberger, P. E., Liu, F., Probst, C. K., Rahimi, R. A., Sakai, N., Kuehl, T., Ryan, J., Bhola, P., et al. (2017). Targeted apoptosis of myofibroblasts with the BH3 mimetic ABT-263 reverses established fibrosis. Sci Transl Med 9. 10.1126/scitranslmed.aa13765.
22. Clark, J. A., Cheng, J. C., and Leung, K. S. (1996). Mechanical properties of normal skin and hypertrophic scars. Burns 22, 443-446. 10.1016/0305-4179(96)00038-1.
23. Ham, H. I., Ogawa, R., Hsu, C. K., Hughes, M. W., Tang, M. J., and Chuong, C. M. (2019). The tension biology of wound healing. Exp Dermatol 28, 464-471. 10.1111/exd.13460.
24. Barrat, F. J., and Lu, T. T. (2019). Role of type I interferons and innate immunity in systemic sclerosis: unbalanced activities on distinct cell types?Curr Opin Rheumatol 31, 569-575. 10.1097/BOR.0000000000000659.
25. Stifano, G., and Christmann, R. B. (2016). Macrophage Involvement in Systemic Sclerosis: Do We Need More Evidence?Curr Rheumatol Rep 18, 2. 10.1007/s11926-015-0554-8.
26. Guiducci, C., Tripodo, C., Gong, M., Sangaletti, S., Colombo, M. P., Coffman, R. L., and Barrat, F. J. (2010). Autoimmune skin inflammation is dependent on plasmacytoid dendritic cell activation by nucleic acids via TLR7 and TLR9. J Exp Med 207, 2931-2942. 10.1084/jem.20101048.
27. Gregorio, J., Meller, S., Conrad, C., Di Nardo, A., Homey, B., Lauerma, A., Arai, N., Gallo, R. L., Digiovanni, J., and Gilliet, M. (2010). Plasmacytoid dendritic cells sense skin injury and promote wound healing through type I interferons. J Exp Med 207, 2921-2930. 10.1084/jem.20101102.
28. Guttman-Yassky, E., Diaz, A., Pavel, A. B., Fernandes, M., Lefferdink, R., Erickson, T., Canter, T., Rangel, S., Peng, X., Li, R., et al. (2019). Use of Tape Strips to Detect Immune and Barrier Abnormalities in the Skin of Children With Early-Onset Atopic Dermatitis. JAMA Dermatol 155, 1358-1370. 10.1001/jamadermatol.2019.2983.
29. van der Molen, R. G., Spies, F., van't Noordende, J. M., Boelsma, E., Mommaas, A. M., and Koerten, H. K. (1997). Tape stripping of human stratum corneum yields cell layers that originate from various depths because of furrows in the skin. Arch Dermatol Res 289, 514-518. 10.1007/s004030050232.
30. Jin, H., Oyoshi, M. K., Le, Y., Bianchi, T., Koduru, S., Mathias, C. B., Kumar, L., Le Bras, S., Young, D., Collins, M., et al. (2009). IL-21R is essential for epicutaneous sensitization and allergic skin inflammation in humans and mice. J Clin Invest 119, 47-60. 10.1172/JCI32310.
31. Sano, S., Chan, K. S., Carbajal, S., Clifford, J., Peavey, M., Kiguchi, K., Itami, S., Nickoloff, B. J., and DiGiovanni, J. (2005). Stat3 links activated keratinocytes and immunocytes required for development of psoriasis in a novel transgenic mouse model. Nat Med 11, 43-49. 10.1038/nm1162.
32. Wojcik, S. M., Bundman, D. S., and Roop, D. R. (2000). Delayed wound healing in keratin 6a knockout mice. Mol Cell Biol 20, 5248-5255. 10.1128/MCB.20.14.5248-5255.2000.
33. Si, Y., Tsou, C. L., Croft, K., and Charo, I. F. (2010). CCR2 mediates hematopoietic stem and progenitor cell trafficking to sites of inflammation in mice. J Clin Invest 120, 1192-1203. 10.1172/JCI40310.
34. Ghosh, A. K., and Vaughan, D. E. (2012). PAI-1 in tissue fibrosis. J Cell Physiol 227, 493-507. 10.1002/jcp.22783.
35. Dupont, S., Morsut, L., Aragona, M., Enzo, E., Giulitti, S., Cordenonsi, M., Zanconato, F., Le Digabel, J., Forcato, M., Bicciato, S., et al. (2011). Role of YAP/TAZ in mechanotransduction. Nature 474, 179-183. 10.1038/nature10137.
36. Chang, L., Azzolin, L., Di Biagio, D., Zanconato, F., Battilana, G., Lucon Xiccato, R., Aragona, M., Giulitti, S., Panciera, T., Gandin, A., et al. (2018). The SWI/SNF complex is a mechanoregulated inhibitor of YAP and TAZ. Nature 563, 265-269. 10.1038/s41586-018-0658-1.
37. Arany, P. R., Flanders, K. C., Kobayashi, T., Kuo, C. K., Stuelten, C., Desai, K. V., Tuan, R., Rennard, S. I., and Roberts, A. B. (2006). Smad3 deficiency alters key structural elements of the extracellular matrix and mechanotransduction of wound closure. Proc Natl Acad Sci USA 103, 9250-9255. 10.1073/pnas.0602473103.
38. Vermeulen, S., Roumans, N., Honig, F., Carlier, A., Hebels, D., Eren, A. D., Dijke, P. T., Vasilevich, A., and de Boer, J. (2020). Mechanotransduction is a context-dependent activator of TGF-beta signaling in mesenchymal stem cells. Biomaterials 259, 120331. 10.1016/j.biomaterials.2020.120331.
39. Zhou, T., Gao, B., Fan, Y., Liu, Y., Feng, S., Cong, Q., Zhang, X., Zhou, Y., Yadav, P. S., Lin, J., et al. (2020). Piezo1/2 mediate mechanotransduction essential for bone formation through concerted activation of NFAT-YAP1-ss-catenin. Elife 9. 10.7554/eLife.52779.
40. Speight, P., Kofler, M., Szaszi, K., and Kapus, A. (2016). Context-dependent switch in chemo/mechanotransduction via multilevel crosstalk among cytoskeleton-regulated MRTF and TAZ and TGFbeta-regulated Smad3. Nat Commun 7, 11642. 10.1038/ncomms11642.
41. Akira, S. (2006). TLR signaling. Curr Top Microbiol Immunol 311, 1-16. 10.1007/3-540-32636-7_1.
42. Kawai, T., and Akira, S. (2007). Signaling to NF-kappaB by Toll-like receptors. Trends Mol Med 13, 460-469. 10.1016/j.molmed.2007.09.002.
43. Uematsu, S., and Akira, S. (2007). Toll-like receptors and Type I interferons. J Biol Chem 282, 15319-15323. 10.1074/jbc.R700009200.
44. Grote, K., Salguero, G., Ballmaier, M., Dangers, M., Drexler, H., and Schieffer, B. (2007). The angiogenic factor CCN1 promotes adhesion and migration of circulating CD34+ progenitor cells: potential role in angiogenesis and endothelial regeneration. Blood 110, 877-885. 10.1182/blood-2006-07-036202.
45. Crowl, J. T., Gray, E. E., Pestal, K., Volkman, H. E., and Stetson, D. B. (2017). Intracellular Nucleic Acid Detection in Autoimmunity. Annu Rev Immunol 35, 313-336. 10.1146/annurev-immunol-051116-052331.
46. Blasius, A. L., and Beutler, B. (2010). Intracellular toll-like receptors. Immunity 32, 305-315. 10.1016/j.immuni.2010.03.012.
47. Barrat, F. J., Meeker, T., Gregorio, J., Chan, J. H., Uematsu, S., Akira, S., Chang, B., Duramad, O., and Coffman, R. L. (2005). Nucleic acids of mammalian origin can act as endogenous ligands for Toll-like receptors and may promote systemic lupus erythematosus. J Exp Med 202, 1131-1139. 10.1084/jem.20050914.
48. Panciera, T., Azzolin, L., Cordenonsi, M., and Piccolo, S. (2017). Mechanobiology of YAP and TAZ in physiology and disease. Nat Rev Mol Cell Biol 18, 758-770. 10.1038/nrm.2017.87.
49. Liu, F., Lagares, D., Choi, K. M., Stopfer, L., Marinkovic, A., Vrbanac, V., Probst, C. K., Hiemer, S. E., Sisson, T. H., Horowitz, J. C., et al. (2015). Mechanosignaling through YAP and TAZ drives fibroblast activation and fibrosis. Am J Physiol Lung Cell Mol Physiol 308, L344-357. 10.1152/ajplung.00300.2014.
50. Lim, K., and Chang, H. I. (2009). O-GlcNAcylation of Sp1 interrupts Sp1 interaction with NF-Y. Biochem Biophys Res Commun 382, 593-597. 10.1016/j.bbrc.2009.03.075.
51. Brightbill, H. D., Plevy, S. E., Modlin, R. L., and Smale, S. T. (2000). A prominent role for Sp1 during lipopolysaccharide-mediated induction of the IL-10 promoter in macrophages. J Immunol 164, 1940-1951.
52. Tone, M., Powell, M. J., Tone, Y., Thompson, S. A., and Waldmann, H. (2000). IL-10 gene expression is controlled by the transcription factors Sp1 and Sp3. J Immunol 165, 286-291. 10.4049/jimmunol.165.1.286.
53. Sleiman, S. F., Langley, B. C., Basso, M., Berlin, J., Xia, L., Payappilly, J. B., Kharel, M. K., Guo, H., Marsh, J. L., Thompson, L. M., et al. (2011). Mithramycin is a gene-selective Sp1 inhibitor that identifies a biological intersection between cancer and neurodegeneration. J Neurosci 31, 6858-6870. 10.1523/JNEUROSCI.0710-11.2011.
54. Lee, T. J., Jung, E. M., Lee, J. T., Kim, S., Park, J. W., Choi, K. S., and Kwon, T. K. (2006). Mithramycin A sensitizes cancer cells to TRAIL-mediated apoptosis by down-regulation of XIAP gene promoter through Sp1 sites. Mol Cancer Ther 5, 2737-2746. 10.1158/1535-7163.MCT-06-0426.
55. Deniaud, E., Baguet, J., Chalard, R., Blanquier, B., Brinza, L., Meunier, J., Michallet, M. C., Laugraud, A., Ah-Soon, C., Wierinckx, A., et al. (2009). Overexpression of transcription factor Sp1 leads to gene expression perturbations and cell cycle inhibition. PLoS One 4, e7035. 10.1371/journal.pone.0007035.
56. Sengupta, S., Shimamoto, A., Koshiji, M., Pedeux, R., Rusin, M., Spillare, E. A., Shen, J. C., Huang, L. E., Lindor, N. M., Furuichi, Y., and Harris, C. C. (2005). Tumor suppressor p53 represses transcription of RECQ4 helicase. Oncogene 24, 1738-1748. 10.1038/sj.onc.1208380.
57. Metzemaekers, M., Gouwy, M., and Proost, P. (2020). Neutrophil chemoattractant receptors in health and disease: double-edged swords. Cell Mol Immunol 17, 433-450. 10.1038/s41423-020-0412-0.
58. Girbl, T., Lenn, T., Perez, L., Rolas, L., Barkaway, A., Thiriot, A., Del Fresno, C., Lynam, E., Hub, E., Thelen, M., et al. (2018). Distinct Compartmentalization of the Chemokines CXCL1 and CXCL2 and the Atypical Receptor ACKR1 Determine Discrete Stages of Neutrophil Diapedesis. Immunity 49, 1062-1076 e1066. 10.1016/j.immuni.2018.09.018.
59. Gur, C., Wang, S. Y., Sheban, F., Zada, M., Li, B., Kharouf, F., Peleg, H., Aamar, S., Yalin, A., Kirschenbaum, D., et al. (2022). LGR5 expressing skin fibroblasts define a major cellular hub perturbed in scleroderma. Cell 185, 1373-1388 e1320. 10.1016/j.cell.2022.03.011.
60. Truchetet, M. E., Brembilla, N.C., and Chizzolini, C. (2023). Current Concepts on the Pathogenesis of Systemic Sclerosis. Clin Rev Allergy Immunol 64, 262-283. 10.1007/s12016-021-08889-8.
61. Frasca, L., and Lande, R. (2020). Toll-like receptors in mediating pathogenesis in systemic sclerosis. Clin Exp Immunol 201, 14-24. 10.1111/cei.13426.
62. Yao, H. P., Zhou, Y. Q., Zhang, R., and Wang, M. H. (2013). MSP-RON signalling in cancer: pathogenesis and therapeutic potential. Nat Rev Cancer 13, 466-481. 10.1038/nrc3545.
63. Huang, L., Fang, X., Shi, D., Yao, S., Wu, W., Fang, Q., and Yao, H. (2020). MSP-RON Pathway: Potential Regulator of Inflammation and Innate Immunity. Front Immunol 11, 569082. 10.3389/fimmu.2020.569082.
64. Sindrilaru, A., Peters, T., Schymeinsky, J., Oreshkova, T., Wang, H., Gompf, A., Mannella, F., Wlaschek, M., Sunderkotter, C., Rudolph, K. L., et al. (2009). Wound healing defect of Vav3−/− mice due to impaired beta2-integrin-dependent macrophage phagocytosis of apoptotic neutrophils. Blood 113, 5266-5276. 10.1182/blood-2008-07-166702.
65. Djalilian, A. R., McGaughey, D., Patel, S., Seo, E. Y., Yang, C., Cheng, J., Tomic, M., Sinha, S., Ishida-Yamamoto, A., and Segre, J. A. (2006). Connexin 26 regulates epidermal barrier and wound remodeling and promotes psoriasiform response. J Clin Invest 116, 1243-1253. 10.1172/JCI27186.
66. Gagnon, L., Leduc, M., Thibodeau, J. F., Zhang, M. Z., Grouix, B., Sarra-Bournet, F., Gagnon, W., Hince, K., Tremblay, M., Geerts, L., et al. (2018). A Newly Discovered Antifibrotic Pathway Regulated by Two Fatty Acid Receptors: GPR40 and GPR84. Am J Pathol 188, 1132-1148. 10.1016/j. ajpath.2018.01.009.
67. Baitsch, D., Bock, H. H., Engel, T., Telgmann, R., Muller-Tidow, C., Varga, G., Bot, M., Herz, J., Robenek, H., von Eckardstein, A., and Nofer, J. R. (2011). Apolipoprotein E induces antiinflammatory phenotype in macrophages. Arterioscler Thromb Vasc Biol 31, 1160-1168. 10.1161/ATVBAHA.111.222745.
68. Liu, M. J., Bao, S., Galvez-Peralta, M., Pyle, C. J., Rudawsky, A. C., Pavlovicz, R. E., Killilea, D. W., Li, C., Nebert, D. W., Wewers, M. D., and Knoell, D. L. (2013). ZIP8 regulates host defense through zinc-mediated inhibition of NF-kappaB. Cell Rep 3, 386-400. 10.1016/j. celrep.2013.01.009.
69. Rodrigues, M., Kosaric, N., Bonham, C. A., and Gurtner, G. C. (2019). Wound Healing: A Cellular Perspective. Physiol Rev 99, 665-706. 10.1152/physrev.00067.2017.
70. Larouche, J., Sheoran, S., Maruyama, K., and Martino, M. M. (2018). Immune Regulation of Skin Wound Healing: Mechanisms and Novel Therapeutic Targets. Adv Wound Care (New Rochelle) 7, 209-231. 10.1089/wound.2017.0761.
71. Atcha, H., Jairaman, A., Holt, J. R., Meli, V. S., Nagalla, R. R., Veerasubramanian, P. K., Brumm, K. T., Lim, H. E., Othy, S., Cahalan, M. D., et al. (2021). Mechanically activated ion channel Piezol modulates macrophage polarization and stiffness sensing. Nat Commun 12, 3256. 10.1038/s41467-021-23482-5.
72. Meli, V. S., Atcha, H., Veerasubramanian, P. K., Nagalla, R. R., Luu, T. U., Chen, E. Y., Guerrero-Juarez, C. F., Yamaga, K., Pandori, W., Hsieh, J. Y., et al. (2020). YAP-mediated mechanotransduction tunes the macrophage inflammatory response. Sci Adv 6. 10.1126/sciadv.abb8471.
73. Song, R., Gao, Y., Dozmorov, I., Malladi, V., Saha, I., McDaniel, M. M., Parameswaran, S., Liang, C., Arana, C., Zhang, B., et al. (2021). IRF1 governs the differential interferon-stimulated gene responses in human monocytes and macrophages by regulating chromatin accessibility. Cell Rep 34, 108891. 10.1016/j.celrep.2021.108891.
74. Saeed, S., Quintin, J., Kerstens, H. H., Rao, N. A., Aghajanirefah, A., Matarese, F., Cheng, S. C., Ratter, J., Berentsen, K., van der Ent, M. A., et al. (2014). Epigenetic programming of monocyte-to-macrophage differentiation and trained innate immunity. Science 345, 1251086. 10.1126/science.1251086.
75. Lin, M. C., Almus-Jacobs, F., Chen, H. H., Parry, G. C., Mackman, N., Shyy, J. Y., and Chien, S. (1997). Shear stress induction of the tissue factor gene. J Clin Invest 99, 737-744. 10.1172/JCI119219.
76. Ma, W. T., Gao, F., Gu, K., and Chen, D. K. (2019). The Role of Monocytes and Macrophages in Autoimmune Diseases: A Comprehensive Review. Front Immunol 10, 1140. 10.3389/fimmu.2019.01140.
77. Denning, N. L., Aziz, M., Murao, A., Gurien, S. D., Ochani, M., Prince, J. M., and Wang, P. (2020). Extracellular CIRP as an endogenous TREM-1 ligand to fuel inflammation in sepsis. JCI Insight 5. 10.1172/jci.insight.134172.
78. Bouchon, A., Facchetti, F., Weigand, M. A., and Colonna, M. (2001). TREM-1 amplifies inflammation and is a crucial mediator of septic shock. Nature 410, 1103-1107. 10.1038/35074114.
79. van den Hoogen, F., Khanna, D., Fransen, J., Johnson, S. R., Baron, M., Tyndall, A., Matucci-Cerinic, M., Naden, R. P., Medsger, T. A., Jr., Carreira, P. E., et al. (2013). 2013 classification criteria for systemic sclerosis: an American College of Rheumatology/European League against Rheumatism collaborative initiative. Arthritis Rheum 65, 2737-2747. 10.1002/art.38098.
80. Du, Y., Ah Kioon, M. D., Laurent, P., Chaudhary, V., Pierides, M., Yang, C., Oliver, D., Ivashkiv, L. B., and Barrat, F. J. (2022). Chemokines form nanoparticles with DNA and can superinduce TLR-driven immune inflammation. J Exp Med 219. 10.1084/jem.20212142.
81. https://sfvideo.blob.core.windows.net/sitefinity/docs/default-source/protocol/alt-r-crispr-cas9-user-guide-ribonucleoprotein-electroporation-amaxa-nucleofector-system6a01611532796e2eaa53ff00001clb3c.pdf?sfvrsn=71c43407_26.
82. Chaudhary, V., Ah Kioon, M. D., Hwang, S. M., Mishra, B., Lakin, K., Kirou, K. A., Zhang-Sun, J., Wiseman, R. L., Spiera, R. F., Crow, M. K., et al. (2022). Chronic activation of pDCs in autoimmunity is linked to dysregulated ER stress and metabolic responses. J Exp Med 219. 10.1084/jem.20221085.
83. Lee, M. J., Byun, M. R., Furutani-Seiki, M., Hong, J. H., and Jung, H. S. (2014). YAP and TAZ regulate skin wound healing. J Invest Dermatol 134, 518-525. 10.1038/jid.2013.339.
84. https://portals.broadinstitute.org/gpp/public/analysis-tools/%20sgrna-design.
85. Chen, S., Zhou, Y., Chen, Y., and Gu, J. (2018). fastp: an ultra-fast all-in-one FASTQ preprocessor. Bioinformatics 34, i884-i890. 10.1093/bioinformatics/bty560.
86. Team, R. C. (2020). R: A language and environment for statistical computing. R Foundation for Statistical Computing, Vienna, Austria. http://www.r-project.org/.
87. Robinson, M. D., McCarthy, D. J., and Smyth, G. K. (2010). edgeR: a Bioconductor package for differential expression analysis of digital gene expression data. Bioinformatics 26, 139-140. 10.1093/bioinformatics/btp616.
88. Gu, Z., Eils, R., and Schlesner, M. (2016). Complex heatmaps reveal patterns and correlations in multidimensional genomic data. Bioinformatics 32, 2847-2849. 10.1093/bioinformatics/btw313.
89. Langmead, B., and Salzberg, S. L. (2012). Fast gapped-read alignment with Bowtie 2. Nat Methods 9, 357-359. 10.1038/nmeth.1923.
90. Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079. 10.1093/bioinformatics/btp352.
91. Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based analysis of ChIP-Seq (MACS). Genome Biol 9, R137. 10.1186/gb-2008-9-9-r137.
92. Robinson, J. T., Thorvaldsdottir, H., Winckler, W., Guttman, M., Lander, E. S., Getz, G., and Mesirov, J. P. (2011). Integrative genomics viewer. Nat Biotechnol 29, 24-26. 10.1038/nbt.1754.
93. Yu, F., Sankaran, V. G., and Yuan, G. C. (2021). CUT&RUNTools 2.0: a pipeline for single-cell and bulk-level CUT&RUN and CUT&Tag data analysis. Bioinformatics 38, 252-254. 10.1093/bioinformatics/btab507.
94. Pongor, L. S., Gross, J. M., Vera Alvarez, R., Murai, J., Jang, S. M., Zhang, H., Redon, C., Fu, H., Huang, S. Y., Thakur, B., et al. (2020). BAMscale: quantification of next-generation sequencing peaks and generation of scaled coverage tracks. Epigenetics Chromatin 13, 21. 10.1186/s13072-020-00343-x.
95. Korsunsky, I., Millard, N., Fan, J., Slowikowski, K., Zhang, F., Wei, K., Baglaenko, Y., Brenner, M., Loh, P. R., and Raychaudhuri, S. (2019). Fast, sensitive and accurate integration of single-cell data with Harmony. Nat Methods 16, 1289-1296. 10.1038/s41592-019-0619-0.
96. Hao, Y., Hao, S., Andersen-Nissen, E., Mauck, W. M., 3rd, Zheng, S., Butler, A., Lee, M. J., Wilk, A. J., Darby, C., Zager, M., et al. (2021). Integrated analysis of multimodal single-cell data. Cell 184, 3573-3587 e3529. 10.1016/j.cell.2021.04.048.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Methods and Materials:

Patients: Participants were recruited from the institutional review board-approved Hospital for Special Surgery Scleroderma (#2014-276). All participants provided written informed consent before enrollment. All patients fulfilled the 2013 ACR/EULAR Classification Criteria for systemic sclerosis (SSc)⁷⁹. Patients were categorized as having diffuse subtype (early diffuse (edSSc) or late diffuse (ldSSc)) SSc according to LeRoy²⁷. Disease duration was defined as the time from the first SSc related symptom apart from Raynaud phenomenon and was classified as early if the disease duration was ≤2 years. The clinical and demographic characteristics of the patients with SSc are described in Table 1.

TABLE 1
Clinical and demographic characteristics of the patients with SSc

| | |
|---|---|
| Age - years, mean (SEM) | 43.3 (7.1) |
| Sex -n, % female | 3, 66.67% |
| Race - number, percentage | 3, 100% White |
| Disease Duration - years, mean (SE [symbol] | 5.0 (2.1) |
| n, % early diffuse, | 1, 33.3% |
| n, % late diffuse | 2, 66.67% |
| MRSS - mean (SEM) | 21.0 (4.5) |
| Autoantibody - n, % scl70, | 2, 66.67% Scl70 |
| n, % RNA Polymerase 3, | 1, 33.33% POL3 |
| n, % negative, | 0, 0% Neg |
| Interstitial lung disease present - n, [symbol] | 3, 100% |
| Pulmonary hypertension present - n, [symbol] | 1, 33.3% |

[symbol]

indicates data missing or illegible when filed

Purification and culture of monocytes from healthy donors and patients: Enriched leukocytes were obtained from the New York Blood Center (Long Island City, NY) under internal Institutional Review Board-approved protocols and all experiments were approved by the Institutional Biosafety Committee (IBC #: 19-0231) from Weill Cornell Medicine. Peripheral blood mononuclear cells (PBMCs) were prepared using Ficoll-Paque density gradient and monocytes were isolated using CD14⁺ positive selection (Miltenyi Biotec:130-050-201) as manual description. Monocytes were cultured at 100,000-200,000 cells per well in a 96-well plate and incubated at 37° C., 5% CO₂ and 95% humidity. Purified monocytes were cultured with human M-CSF (20 ng/ml) in 96-cell culture plate with various stiffness ranging from 0.2 kPA to 50 kPA purchased from Matrigen (Catalog No: SW96-HTS-COL-PK). For TLR4 or TLR8 activation, monocytes were stimulated with LPS (10 ng/ml) or ORN8-L(130 ng/ml) respectively. In some culture conditions, cells were cultured with Mithramycin-A (CaymanChem:11434), Yodal (Medchemexpress: HY-18723), and Dookul (Medchemexpress: HY-126010).

Mice protocol and Tape stripping mice model: All animal procedures were performed in accordance with the regulations of the Institutional Animal Care and Use Committee of the Hospital for Special Surgery and Weill Cornell Medical College. Tape stripping was performed after shaving the dorsal area (3×3 cm) and stripping the skin with 10 successive freshly cut 2-3 cm pieces of 3M duct tape on the back of mice, as we have previously described⁸⁰. PBMC were collected from blood, followed by RBC lysis. Skin was collected after 1-days of tape stripping for RNA isolation, flow cytometer and cell sorting, and histology. Cell suspensions for flow cytometer and cell sorting were prepared by mincing skin, followed by digestion with a collagenase (Sigma-Aldrich: SCR103; concentration:12.5 mg/ml) and liberase TM (Roche: 05401119001; concentration:100 g/ml) diluted in 1 ml DMEM for 1.5 h at 37° C. The remaining tissue was filtered through a 70-μm strainer (VWR) followed by a 40-μm strainer. Cellular skin infiltrates were characterized by flow cytometry. Following markers were used to identify neutrophils (CD45⁺CD11b⁺Ly6G⁺), Macrophages (CD45⁺CD11b⁺Ly6G⁻F4/80⁺), inflammatory monocytes (CD45⁺CD11b⁺Ly6G⁻Ly6C⁺), and infiltrating monocytes (CD45⁺CD11b⁺Ly6G⁻Ly6C⁺CCR2^{low/high}). For SP1 inhibition, 8-10 weeks old C57BL/6 mice were administered by intraperitoneal injection for consecutive 3 days with 100 μl of vehicle (PBS) or mithramycin-A (0.5 mg/kg) before tape stripping.

Flow cytometry and cell sorting: For surface staining or cell sorting, cells were stained with human FcR blocker (Miltenyi Biotec: 130-059-901) or mouse CD16/32 (Biolegend: 101302) with surface marker antibodies: human CD86 (BD Bioscience: 562432), mice CD45 (Biolegend: 103106), mice CD11b (Biolegend: 101216), mice Ly6G (BD Bioscience:560599), mice CCR2 (Biolegend: 150615), Ly6C (Biolegend: 128011), CD86 (Biolegend: 105006), F4/80(eBioscience: 48-4801-82) in 1:100 dilution in FACS buffer for 30 min at 4′C. For cell viability, cells were stained in FACS buffer with DAPI or Zombie UV™ fixable dye (Biolegend: 423107) in PBS. Cells were acquired by a fluorescence activated cell sorter (FACS) and analysis was performed using FlowJo analysis software.

RNA extraction and RT-PCR: Monocytes from the ex-vivo culture, cell sorting, or total skin were lysed for total RNA extraction using the Qiagen RNeasy Plus Mini Kit. cDNA were prepared using high-capacity cDNA Reverse Transcription kit (Thermofisher: 4368813). qPCR reactions were performed, and gene expression levels were calculated based on relative threshold cycle (Ct) values as described 4. This was done using the formula Relative Ct=1000×1.8 (HSK-GENE), where HSK is the mean CT of duplicate housekeeping gene runs (GAPDH), GENE is the mean CT of duplicate runs of the gene of interest, and 1000 is arbitrarily chosen as a factor to bring all values above 0. Primers are given in Table 2.

TABLE 2
List of primers

| | Forward | Reverse |
|---|---|---|
| | Primer | Primer |
| Genes | Sequences | sequence |
| Human GAPDH | ATCAAGAAGG | GTCGCTGTTGA |
| | TGGTGAAGCA | AGTCAGAGGA |
| | (SEQ ID NO: 1) | (SEQ ID NO: 2) |
| Human IL-6 | TACCCCCAGG | GCCATCTTTG |
| | AGAAGATTCC | GAAGGTTCAG |
| | (SEQ ID NO: 3) | (SEQ ID NO: 4) |
| Human TNF-α | CTTCTGCCTG | CTGGGCCAGA |
| | CTGCACTTTG | GGGCTGAT |
| | (SEQ ID NO: 5) | (SEQ ID NO: 6) |
| Human IL-1β | TTCGACACAT | TTTTTGCTGT |
| | GGGATAACGA | GAGTCCCGGA |
| | GG | G |
| | (SEQ ID NO: 7) | (SEQ ID NO: 8) |
| Human CXCL8 | GCTCTAGAAT | CGGGATCCTT |
| | GACTTCCAAG | ATGAATTCTC |
| | CTGGCCG | AGCCCTC |
| | (SEQ ID NO: 9) | (SEQ ID NO: 10) |
| Human SP1 | CATACAGGCG | GTGCCTCTGT |
| | AGAGGCCATT | AGCTCATCCG |
| | (SEQ ID NO: 11) | (SEQ ID NO: 12) |
| Human VAV3 | CCAACCCTGG | CCTGTGCCTC |
| | TATGCTGGAG | ACAAGGTAAG |
| | | T |
| | (SEQ ID NO: 13) | (SEQ ID NO: 14) |
| Mouse TNF-α | GGTCTGGGCC | GCCACCACGC |
| | ATAGAACTGA | TCTTCTGTCT |
| | TG | |
| | (SEQ ID NO: 15) | (SEQ ID NO: 16) |
| Mouse IL-1β | AAACCGTTTT | GACGGCACAC |
| | TCCATCTTCT | CCACCCT |
| | TCTTT | |
| | (SEQ ID NO: 17) | (SEQ ID NO: 18) |
| Mouse IL-6 | GAGGATACCA | AAGTGCATCA |
| | CTCCCAACAG | TCGTTGTTCA |
| | AC | TA |
| | (SEQ ID NO: 19) | (SEQ ID NO: 20) |
| Mouse IP-10 | GCTTCCCTAT | GACGGTCCGC |
| | GGCCCTCATT | TGCAACTG |
| | (SEQ ID NO: 21) | (SEQ ID NO: 22) |
| Mouse CXCL2 | CCAACCACCA | GCGTCACACT |
| | GGCTACAGG | CAAGCTCTG |
| | (SEQ ID NO: 23) | (SEQ ID NO: 24) |
| Mouse GAPDH | TGCACCACCA | GGATGCAGGG |
| | ACTGCTTAG | ATGATGTTC |
| | (SEQ ID NO: 25) | (SEQ ID NO: 26) |

Chemokine and Cytokine measurement: Secreted cytokines such as TNF-α (Mabtech: 3512-1A-6), IL-1β (Mabtech: 3416-2H) and IL-6 (Mabtech: 3460-1H-20) were quantified in the supernatant of monocytes cultures using enzyme-linked immunosorbent assay (ELISA) according to the manufacturer's protocol.

Gene editing in human monocytes: Human monocytes isolated from PBMCs were transfected by adding 150 nM sgRNA-CAS9 ribonucleoprotein complexes to 10⁵ cells in suspension using the Lipofectamine™ CRISPRMAX reagent (Thermofisher: 13778075). All materials for sgRNA-Cas9 complex generation were purchased from Integrated DNA Technologies and prepared as instructed^{81,82}. Eighty hours post-transfection, genetic ablation of target genes was assessed via quantitative RT-PCR. The 20-nucleotide CRISPR-RNA (crRNA) targeting human SP1 (Homo sapiens chromosome 22, GRCh38.p12, NM_138473.3) is directed at the genomic sequence 5′-CCATCAACGGTCTGGAACTGTGG-3′ (SEQ ID NO:27) and human VAV3 (Homo sapiens chromosome 22, GRCh38.p12, NM_006113.5) is directed at the genomic sequence 5′-ACGGCCTGTTGTGAGACGTTTGG-3′ (SEQ ID NO:28) (the 3 additional nucleotides highlighted in bold represent the protospacer adjacent motif, or PAM). These target sequences correspond to exon 4 of the human SP1 transcript and exon 2 of the human VAV3 transcript and was manually chosen by identifying a 20-base pair fragment immediately upstream of the highlighted PAM⁸³. The most likely on- and off-target effects of the manually selected CRISPR sequence were then analyzed using the Broad Institute's Genetic Perturbation Platform⁸⁴. To validate the genomic editing capacity of the crRNA, quantitative RT-PCR was performed on total RNA isolated from cells transfected with sgRNA-Cas9 complexes containing the SP1 or VAV3 crRNA as described above. The primers for evaluating deletion efficacy are listed in Table 2.

RNA-sequencing and analysis: Total RNA was extracted from cells using the Qiagen RNeasy Plus Mini Kit. All samples were examined for RNA quality by Agilent Bioanalyzer 2100. Illumina libraries were constructed using NEB low input library preparation kit. Multiplexed libraries were generated and pooled at equimolar concentration, and pair-end reads were sequenced on an Illumina NOVASeq 6000 in the Weill Cornell Epigenomics Core Facility at the depth of 21-37 million fragments per sample. Sequencing quality was measured with fastp⁸⁵. Reads mapped in genes were counted against the human genome (hg38) with STAR aligner and Gencode v21. Differential gene expression analysis was performed in R⁸⁶ using the edgeR package^{87,88}. Genes with low expression levels (<3 cpm) were filtered from all downstream analyses. The Benjamini-Hochberg false discovery rate procedure was used to calculate the FDR. Genes with FDR<0.05 and log 2 (fold-change)>1 were considered significant. Heatmap were generated by Morpheus packages. Pathways analysis for differential regulated genes (DEGs) were performed in Qiagen Ingenuity Pathway analysis (IPA) and z-scores or p-value were plotted in Prism 9 where z-score≤2 represents inhibition and z-score≥2 represents activation.

ATAC-Sequencing and analysis: ATAC-seq library preparation was performed as previously described (Corces et al., 2017). Briefly, 2×10⁵ monocytes were cultured with stiffness 0.2 kPA and 50 kPA for 6 h. Cells were harvested immediately for ATAC-seq library construction. Libraries from each condition were pooled together based on molecular concentration before sequencing using the Illumina NOVASeq 6000 platform. ATAC-seq fastq files were processed and analyzed according to ENCODE best practices. Briefly, reads were checked for quality, and adapters were trimmed using fastp. Reads passing quality control were aligned to the latest release of the human genome (hg38) with the Bowtie2 aligner⁸⁹. Reads aligning in proper pairs to a single location in the genome were further filtered to exclude PCR duplicates using Samtools⁹⁰. High-quality, deduplicated reads were used to call peaks with Macs2⁹¹. Peaks that were observed in at least two replicates were used for differential peak analysis with edgeR⁸⁷. HOMER was used to identify both known and De novo motifs in the sets of differential motifs. IGV browser from Broad Institute is used to visualize the normalized bigwig files⁹². JASPAR motif were performed in R using HSSscript package developed by Hospital for Special surgery Genomic Research Center (https://gitlab.com/hssgenomics/Shiny-ATAC).

CUT&RUN sequencing and analysis: CUT&RUN-seq library preparation was performed as per the protocol (14-1048). Briefly, 2.5×10⁵ purified monocytes were cultured were cultured with stiffness 0.2 kPA or 50 kPA for 18 h. Cells were harvested immediately for CUT&RUN-seq library construction. For the CUT&RUN assay, we followed the manufacturer's protocol using CUT&RUN kit (EpiCypher catalog No: 14-1048) using SP1 antibody (Bethyl Labs: A300-134AT). To create DNA libraries, we utilized fragmented DNA obtained from the CUT&RUN assay and processed it with the NEBNext Ultra II DNA Library Prep Kit for Illumina (New England Biolabs), adhering to the manufacturer's instructions. Finally, the samples were sequenced using a pair-end 50 bp sequencing in Illumina NOVASeq-6000 platform and analyzed using CUT&RUNTools 2.0⁹³ Briefly, the sequenced reads were aligned to the human genome (hg38) using bowtie2⁸⁹. And normalized coverage bigwig files relative to the sequencing depth were generated using BAMscale⁹⁴. IGV browser from Broad Institute is used to visualize the normalized bigwig files⁹².

sc-RNA sequencing analysis: The original sc-RNA-Seq data of each sample from GSE19545259 were combined and batch corrected by Harmony⁹⁵ and analyzed in SeuratV4⁹⁶. The cell assignment and patient information were based on the metadata uploaded by the inventors⁵⁹ Infiltrating Inflammatory monocytes cluster were the combination of the Mo, Mo_CD16, M_IL1B, and M_CD16_IL1B cells. Violin plot showing the IL-1β, TNF-α, CXCL8 and CCL4 expression in blood and skin tissues of inflammatory monocytes cells from SSc patients.

Statistical analysis: All statistical analyses were performed using GraphPad Prism 9 software. Comparisons between two groups were assessed using unpaired or paired (for matched comparisons) two-tailed Student's t-test, or non-parametric Mann-Whitney U-test, or one-way ANOVA with Turkey's correction, wherever applicable. Each dot indicates an individual donor. Data are presented as mean±sem. P values of <0.05 were considered to be statistically significant.

### Example 1. Skin Microenvironment Trigger Inflammatory Response in Infiltrating Monocytes

8-10 weeks old C57BL/6 mice were either shaved only or tape stripping (TAPE). After day 1, blood and skin biopsies were collected.

FIGS. 1A-1, 1A-2, 1A-3, 1A-4, and 1A-5 show the total RNA of skin isolated from skin biopsies and analyzed for inflammatory genes such as TNF-α, IL-1b, IP-10 and IL-6 expression. FIG. 1B shows the infiltration of CD45⁺ cells in inflamed skin analyzed via flow cytometer. FIG. 1C shows the infiltration of neutrophils (CD45⁺CD11b⁺Ly6G⁺) in inflamed skin analyzed via flow cytometer. FIG. 1D shows the infiltration of macrophages (CD45⁺CD11b⁺Ly6G⁻F480⁺) in inflamed skin analyzed via flow cytometer. FIG. 1E shows the infiltration of inflammatory monocytes (CD45⁺CD11b⁺Ly6G⁻Ly6C⁺) in inflamed skin analyzed via flow cytometer. FIGS. 1F and 1G show infiltrating monocytes (CD45⁺CD11b⁻Ly6G⁻Ly6C⁺CCR2^{low/high}) in inflamed skin analyzed via flow cytometer. FIG. 1H shows CD86 expression on infiltrating monocytes in inflamed skin analyzed via flow cytometer.

FIGS. 1I-1, 1I-2, 1I-3, and 1I-4 show results with 8-10 weeks old C57BL/6 mice preformed with tape stripping (TAPE). Infiltrating monocytes were sorted from blood or inflamed skin after day 1 of TAPE and collected for RNA. Gene expressions for inflammatory cytokines such as IL-1b, IL-6, TNF-a, and CXCL2 were analyzed.

Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

### Example 2. Stiffness-Mediated Mechanosensing Trigger Inflammatory Responses in Monocytes Via TREM1 and GPR30 Receptor

Monocytes from freshly isolated blood from Healthy Donor (HD) were cultured with increasing stiffness. Supernatant were collected at different time points and analyzed for secretion for TNF-α, IL-1β and IL-6 via ELISA. FIG. 2A shows the results for TNF-α secretion. FIG. 2B shows the results for IL-1β secretion. FIG. 2C shows the results for IL-6 secretion. FIG. 2D shows CD86 expression levels assessed after 24 h of culture via flow cytometry.

Monocytes were cultured with increasing stiffness for 24 h and washed with fresh media. Then the cells were treated with TLR4 ligand (LPS:10 ng/ml) or TLR8 ligand (ORN8L:130 μg/ml) for 6 h. Supernatant were collected and analyzed for secretion for TNF-α, IL-1β and IL-6 via ELISA. FIG. 2E shows the results for IL-1β secretion. FIG. 2F shows the results for IL-6 secretion. FIG. 2G shows the results for TNF-α secretion.

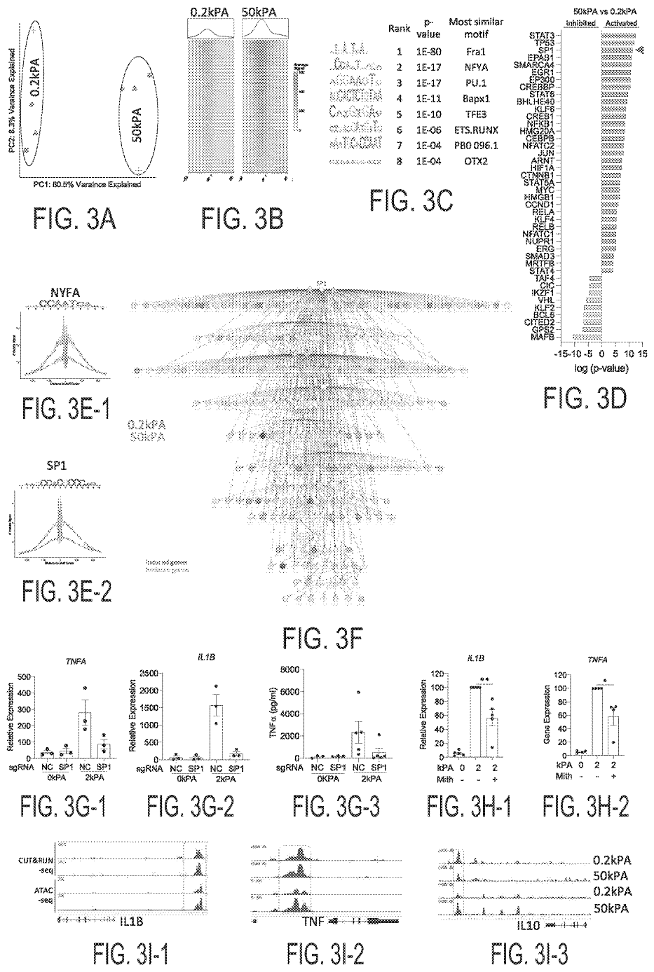

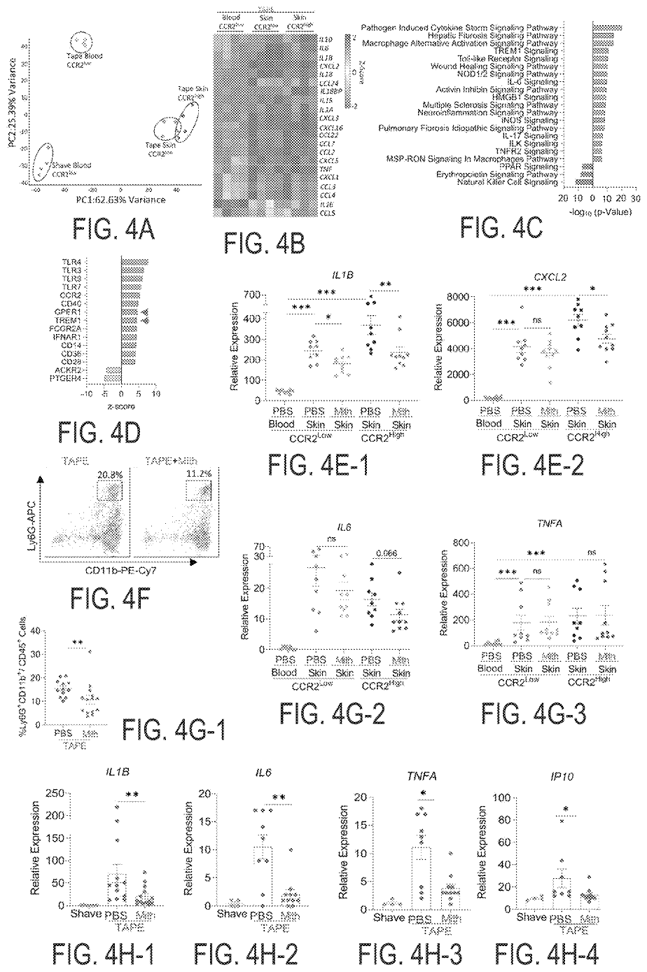

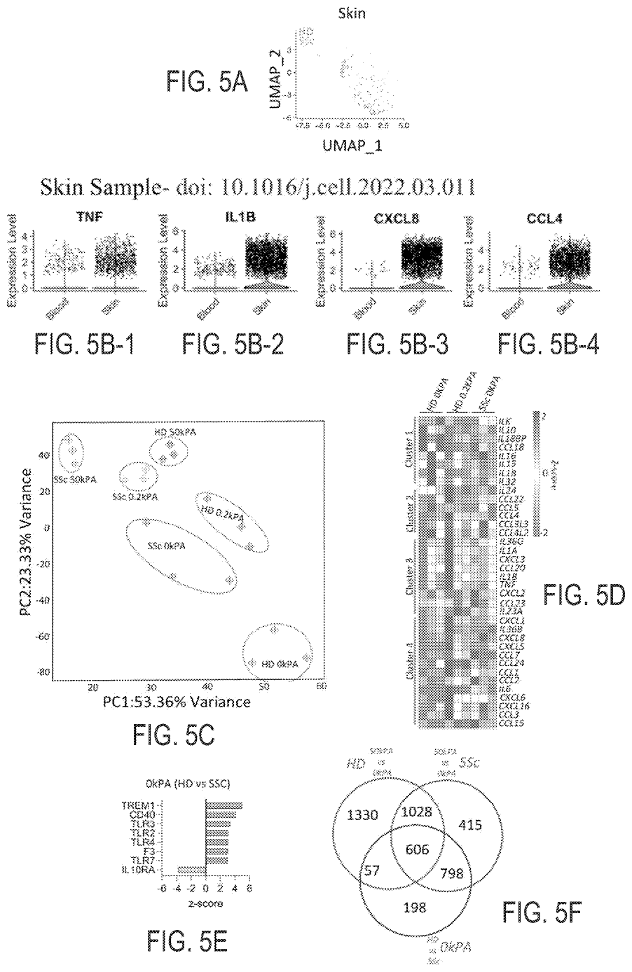

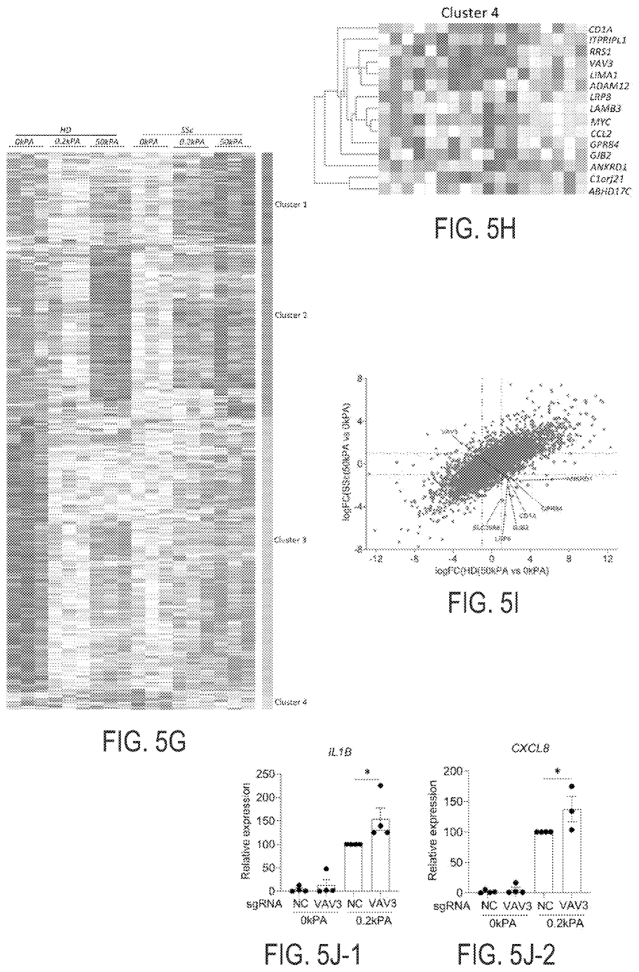

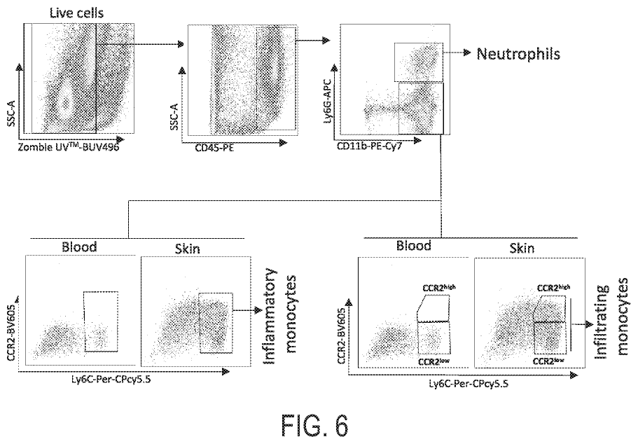

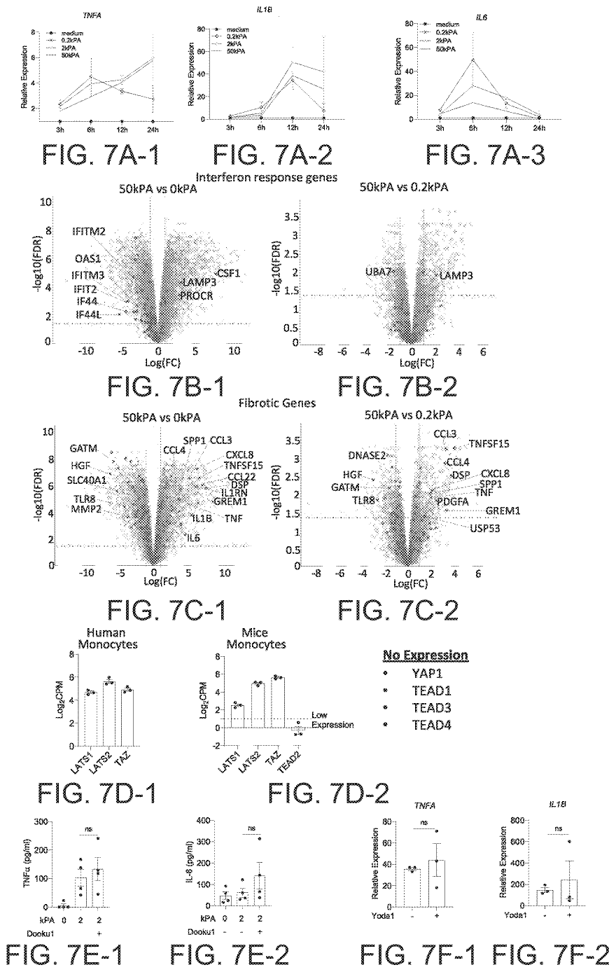

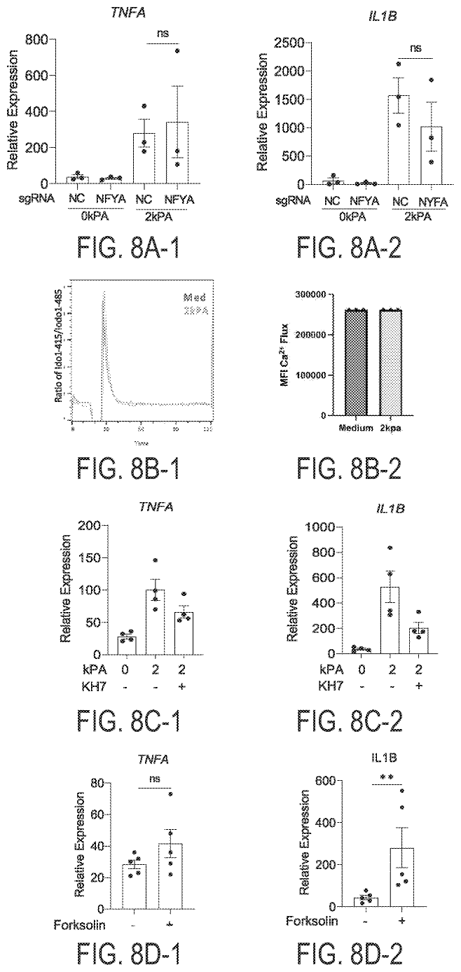

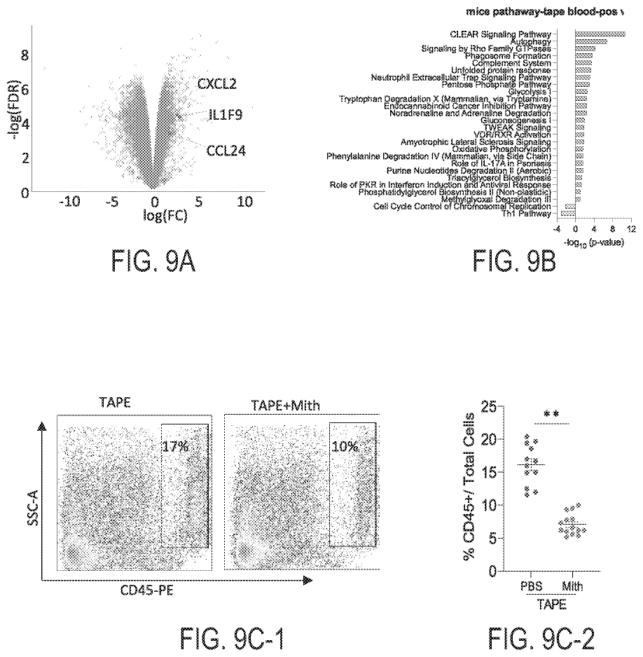

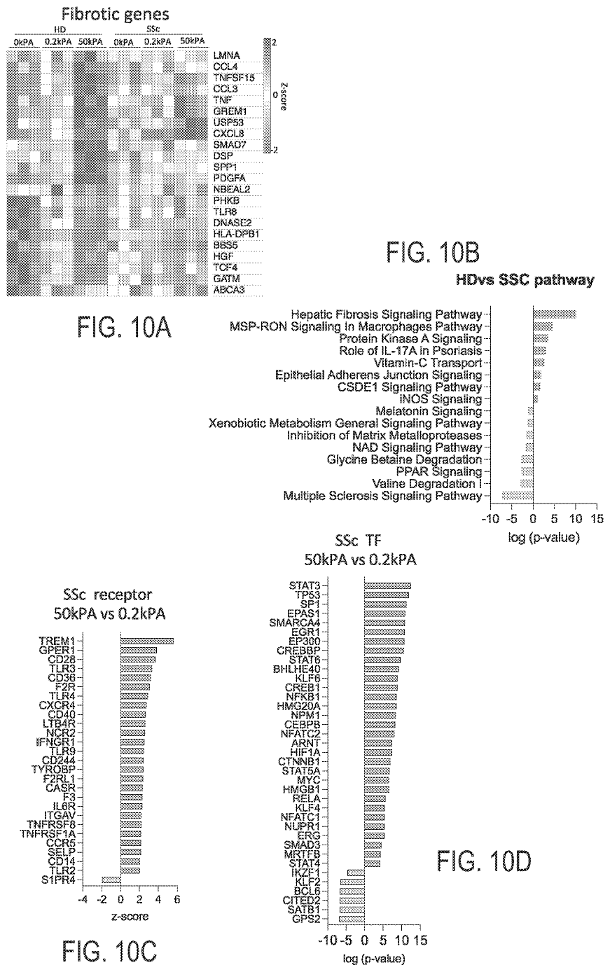

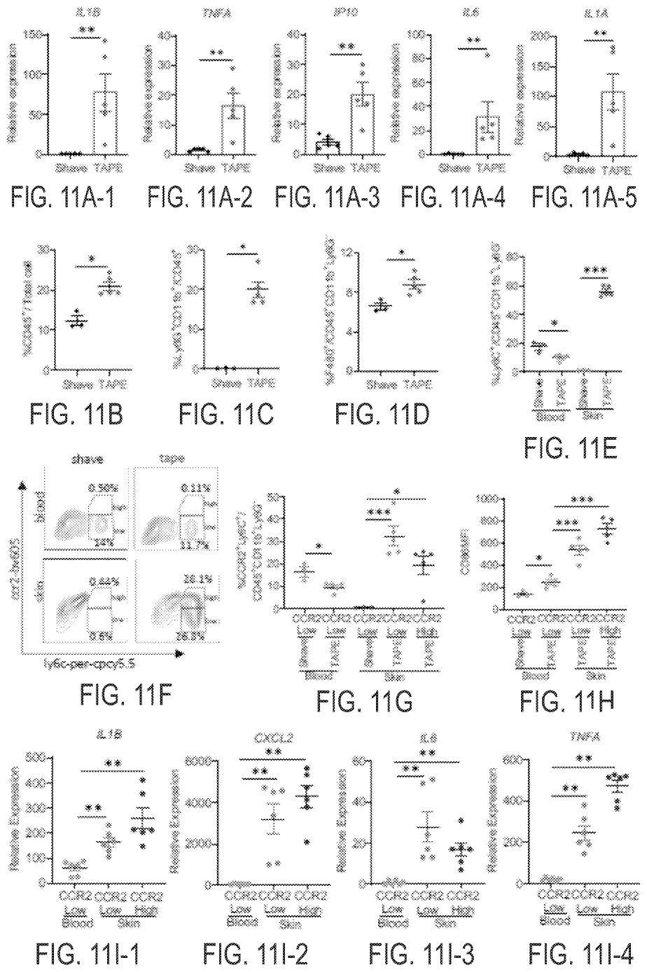

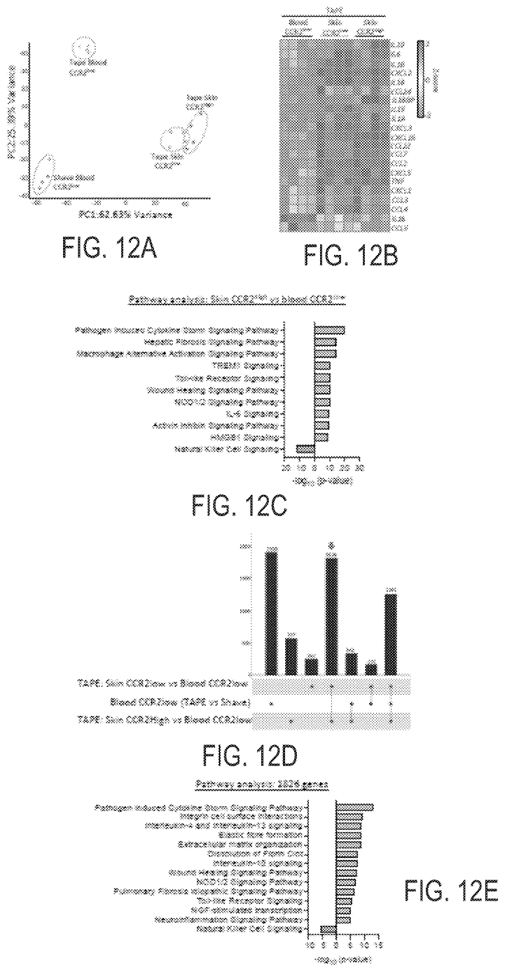

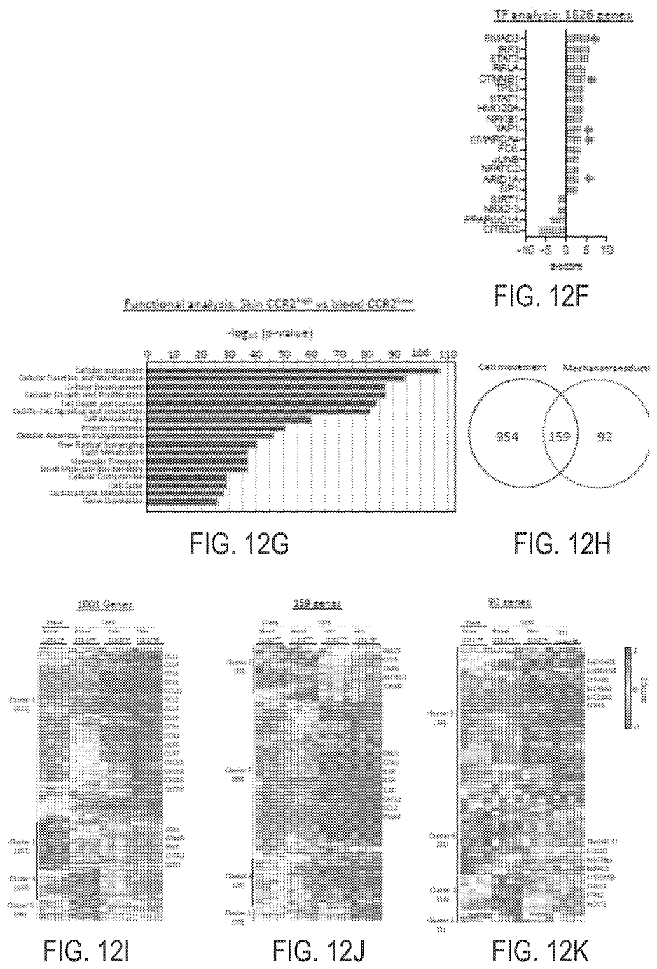

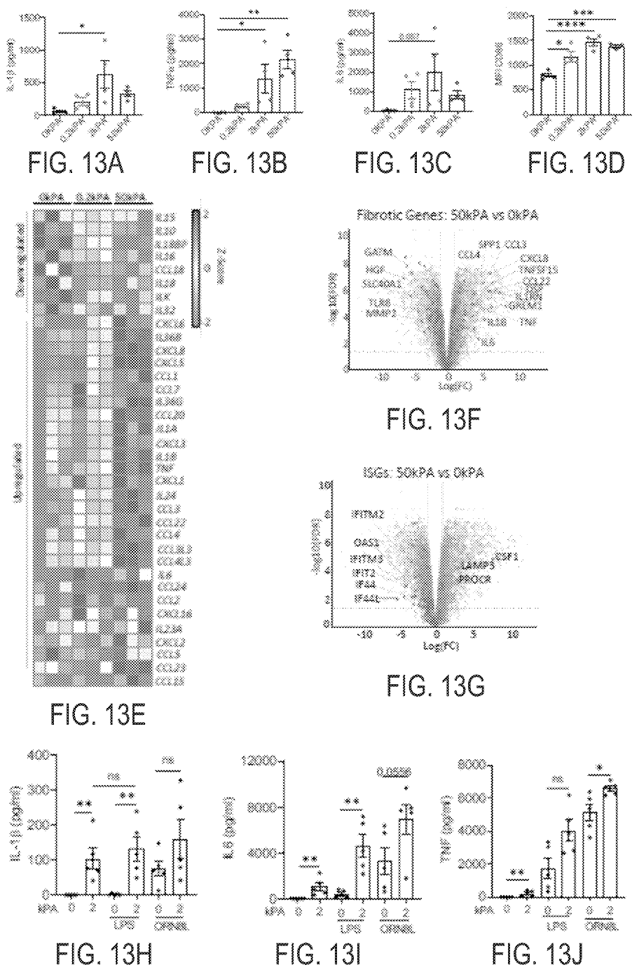

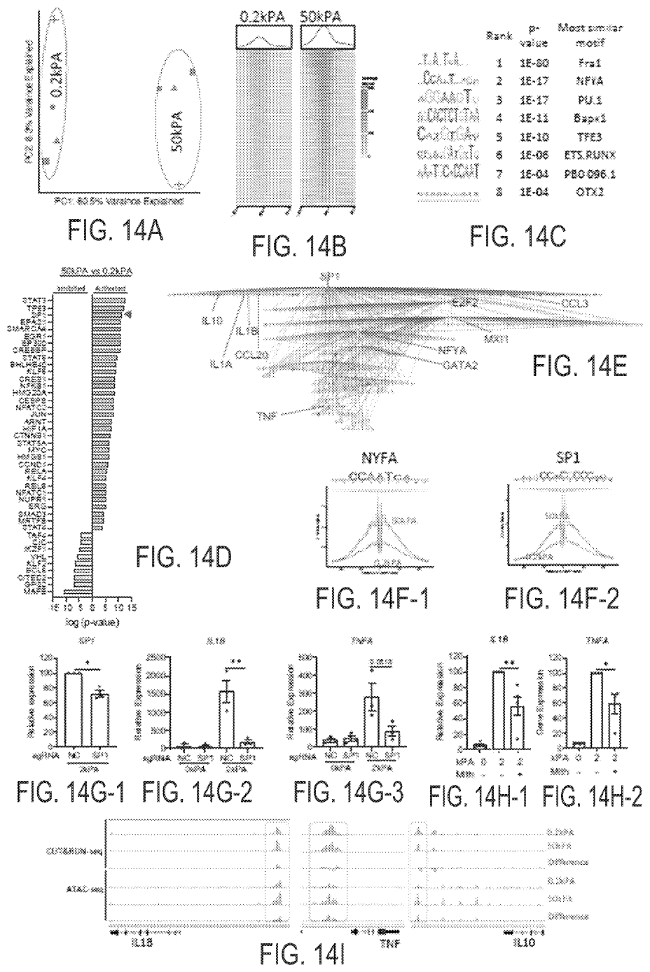

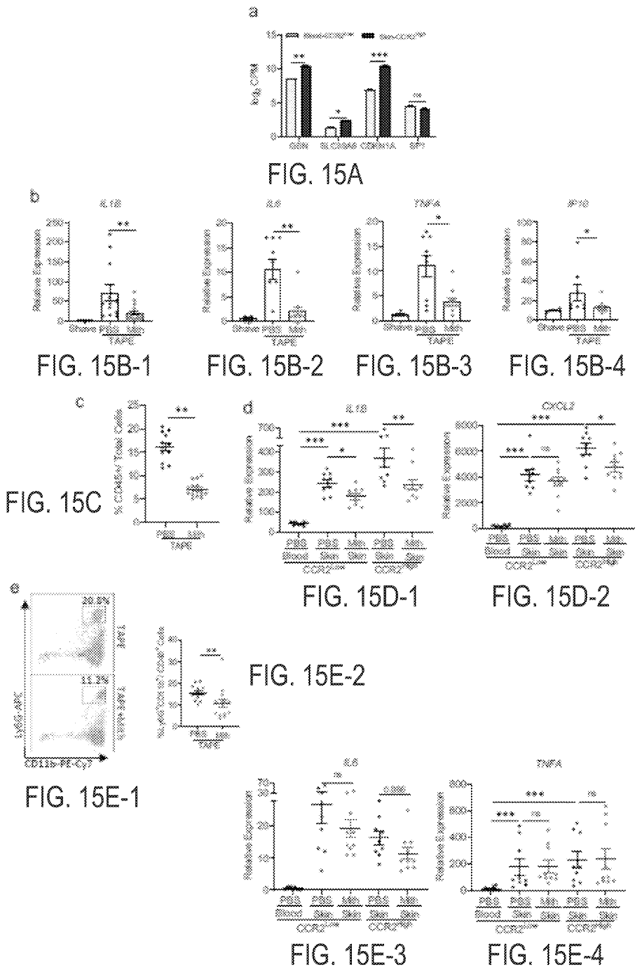

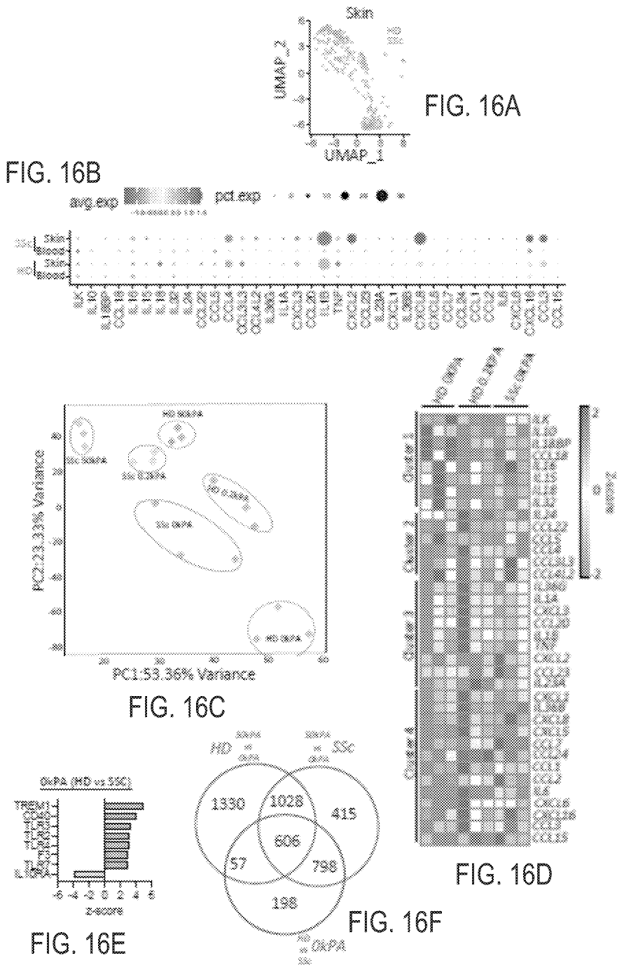

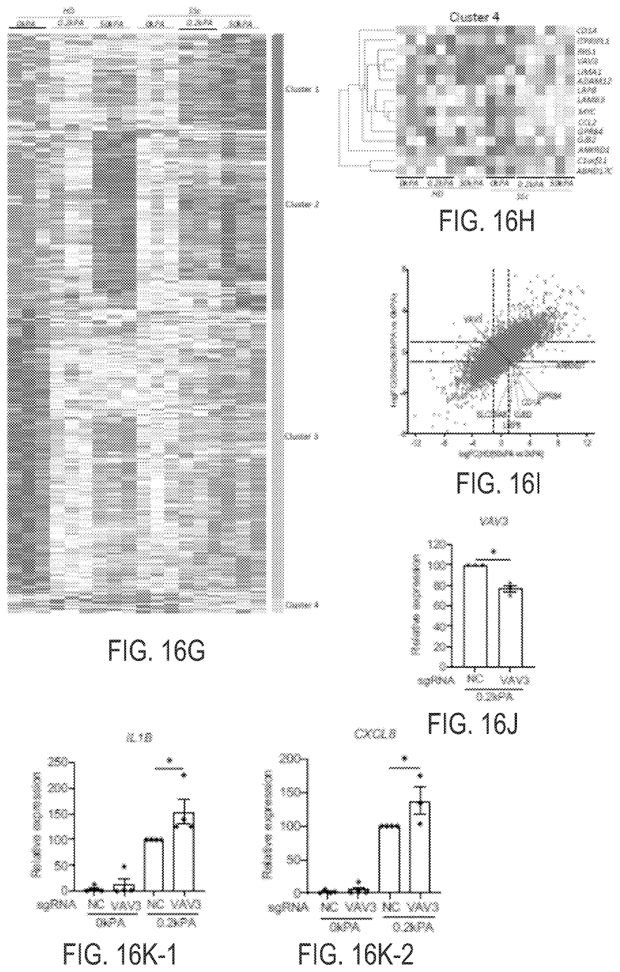

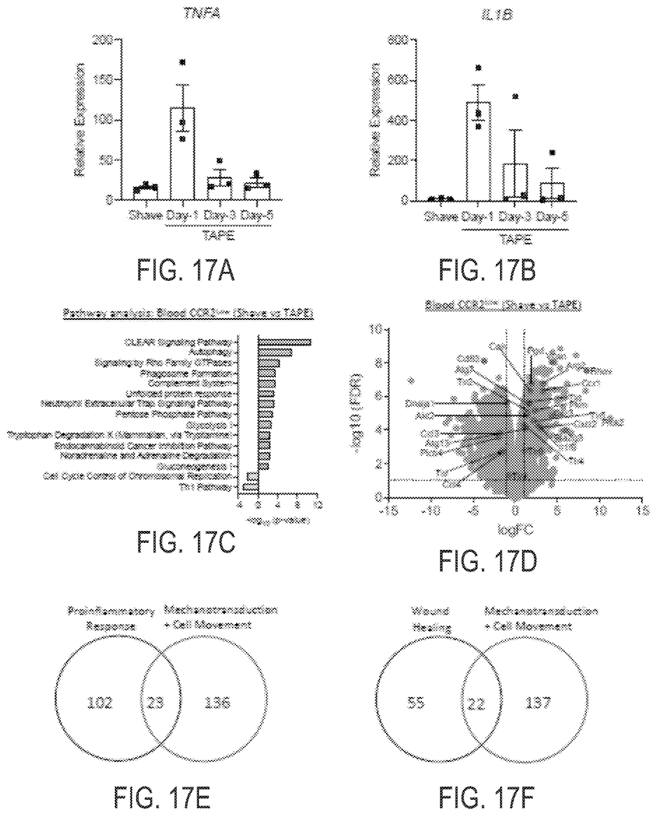

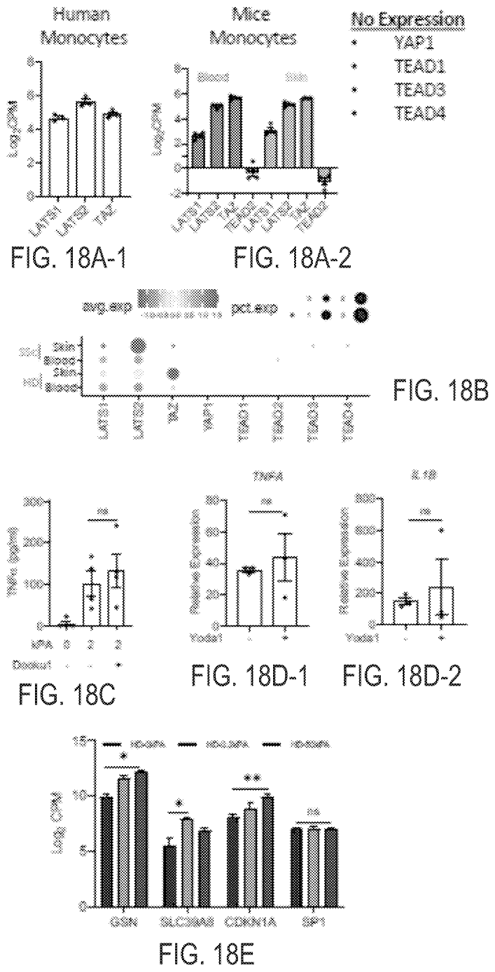

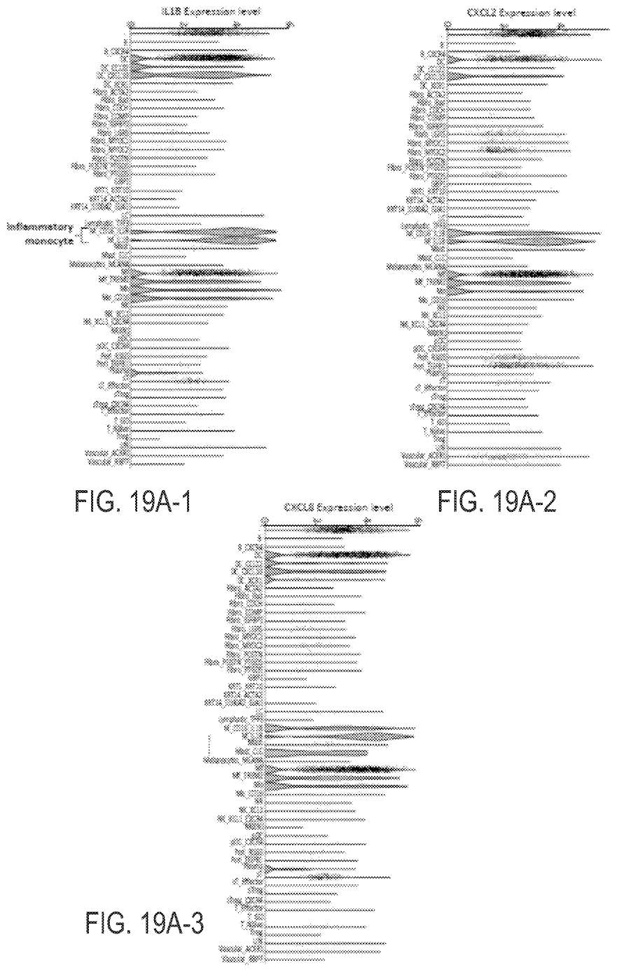

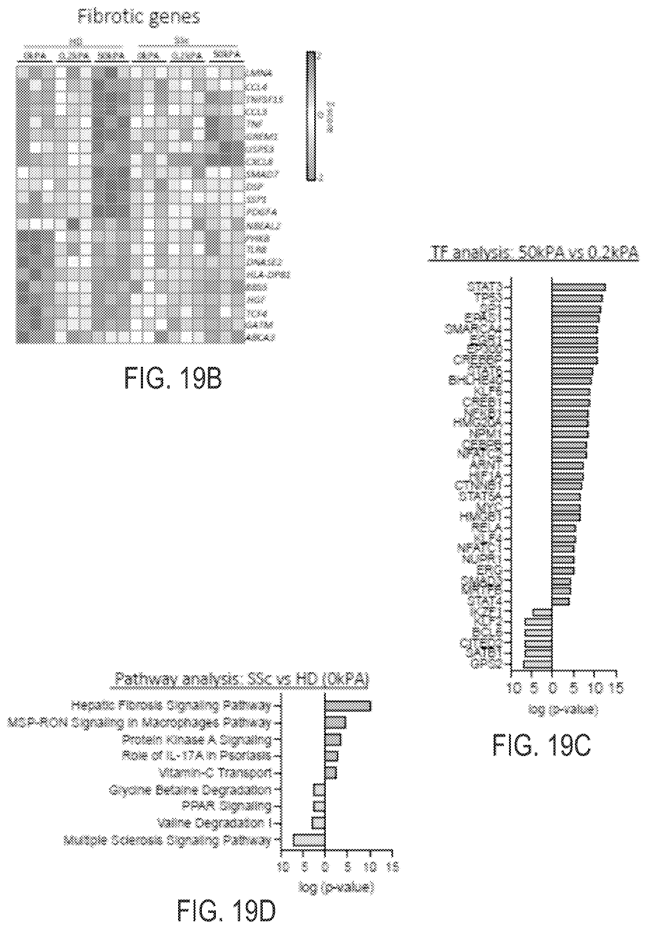

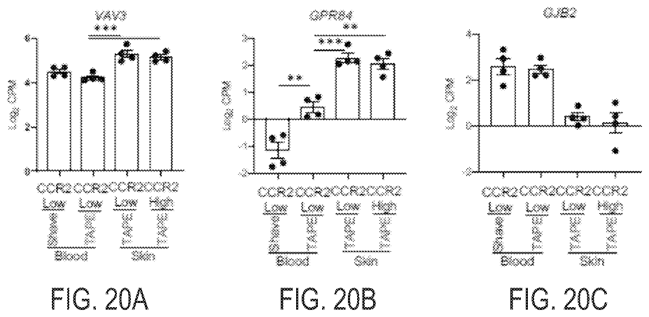

Monocytes were cultured with increasing stiffness and RNA were collected after 24 h and preformed RNA-sequencing. FIG. 2H shows heatmaps of the inflammatory cytokines that are activated by stiffness. FIG. 21 shows results of DEGs analyzed for pathways that are impacted by stiffness using QIAGEN Ingenuity Pathway Analysis. FIG. 2J shows results of DEGs analyzed for receptors that are impacted by stiffness using QIAGEN Ingenuity Pathway Analysis.

Monocytes were cultured in media alone or with antibody for TREM1 (5 or 10 μg/ml) for 6 h. Supernatant were collected and analyzed for TNF-α secretion via ELISA. FIG. 2K shows the TNF-α secretion results.

Monocytes were transfected with CRISPR-Cas9 complex targeting for TREM1 using Lipofectamine™ CRISPRMAX reagent. After transfection, monocytes were cultured with baricitinib (1 μM) for 24 h, followed by culturing in fresh media for 48 h, and then culturing at 2kPA stiffness for 6 h. RNA were collected and analyzed for TNF-α expression by Q-PCR. FIG. 2L shows the TNF-α expression results.

Monocytes were cultured in media alone or with selective GPR30 inhibitor (G15) for 1 h, followed by cultured in either 0 kPA or 2 kPA stiffness for 6 h. RNA were collected and analyzed for the expression of TNF-α and IL-1β by Q-PCR. FIGS. 2M-1 and 2M-2 show the TNF-α and IL-1p expression results.

Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

### Example 3. Stiffness-Mediated Mechanosensing Activates Transcription Factor SP1 Inducing Inflammatory Genes in Monocytes

Monocytes were cultured at either low stiffness (0.2 kPA) or high stiffness (50 kPA) for 6 h and collected for ATAC-sequencing. FIG. 3A shows a PCA plot of ATAC-seq analysis. FIG. 3B shows a heatmap of differentially peaks by stiffness. FIG. 3C shows a de novo motif analysis of differentially peaks by various stiffness using HOMER.

Monocytes from freshly isolated blood from Healthy Donor (HD) were cultured with increasing stiffness. RNA were collected after 24 h and preformed RNA-sequencing. All differential regulated genes (DEGs) were analyzed for transcriptional factors that are activated and inhibited by stiffness using QIAGEN Ingenuity Pathway Analysis as shown in FIG. 3D.

FIGS. 3E-1 and 3E-2 show peak analysis of NFYA and SP1 on cultured monocytes at either low or high stiffness.

Monocytes were transfected with CRISPR-Cas9 complex targeting for SP1 on using Lipofectamine™ CRISPRMAX reagent. After transfection, monocytes were cultured for 72 h, followed by 2 kPA stiffness for 6 h. RNA were collected and analyzed for SP1, TNF-α, and IL-1β expression as shown in FIG. 3F.

Monocytes were cultured in media alone or with mithramycin for 1 h, followed by cultured in either 0 kPA or 2 kPA stiffness for 6 h. RNA were collected and analyzed for TNF-α, IL-1β and IL-6 expression as shown in FIGS. 3G-1, 3G-2, 3G-3, 3H-1, and 3H-2.

Monocytes were cultured at either low stiffness (0.2 kPA) or high stiffness (50 kPA). Cell were collected after 24 h for CUT&RUN assay as shown in FIGS. 3I-1, 3I-2, and 3I-3.

Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

S

### Example 4. SP1-Mediated Mechanosensing Modulates Inflammation

8-10 weeks old C57BL/6 mice were either shaved or tape stripped (TAPE). After day 1, blood and skin were collected for cell sorting to isolate infiltrating monocytes (CD45⁺CD11b⁻Ly6G⁻Ly6C⁺CCR2^{low/high}) RNA were collected from the sorted cells and preformed RNA-sequencing. FIG. 4A shows PCA plot of RNA-seq analysis. FIG. 4B shows heatmaps of inflammatory cytokines on infiltrating monocytes from blood and inflamed skin. FIGS. 4C and 4D show analysis of DEGs pathways (FIG. 4C) and receptors (FIG. 4D) that are impacted by skin-stiffness using QIAGEN Ingenuity Pathway Analysis.

8-10 weeks old C57BL/6 mice were administrated with either PBS or Mithramycin-A (0.5 mg/kg) for consecutive 3 days via intraperitoneal (IP) injection and preformed tape stripping (TAPE). Infiltrating monocytes were sorted from blood or inflamed skin after day 1 of TAPE and collected for RNA. Gene expressions for inflammatory cytokines such as IL-1β, IL-6, TNF-α, and CXCL2 were analyzed as shown in FIGS. 4E-1 and 4E-2.

8-10 weeks old C57BL/6 mice were administrated with either PBS or Mithramycin-A (0.5 mg/kg) for consecutive 3 days via intraperitoneal (IP) injection. Then mice were preformed either shaved or tape stripping (TAPE). After 1 day of TAPE, blood and skin biopsies were collected for flow cytometer and analyzed the infiltration of neutrophils as shown in FIG. 4F. After 1 day of TAPE, skin were collected for RNA isolation and H&E staining for the data in FIGS. 4G and 4H. Gene expression for inflammatory genes such as TNF-α, IL-1β, IP-10, and IL-6 expression were analyzed. Individual mice are indicated as shown in FIGS. 4G-1, 4G-2, 4G-3, 4H-1, 4H-2, 4H-3, and 4H-4; all results are represented as mean±SEM; and statistical significance was evaluated using Mann-Whitney U test or one-way ANOVA, Tukey's multiple comparisons test. ns, P>0.05; *, P<0.05; ***P<0.001.

### Example 5. Mechanosensing Activates Distinct Genes in Infiltrating Macrophages of SSc to Promote Chronic Inflammation

Single cell RNA-sequencing (sc-RNA-seq) were re-analyzed for infiltrating monocytes from published dataset (Gur et al., 2022, Cell; doi: 10.1016/j.cell.2022.03.011). FIG. 5A shows a U-map plot of monocytes infiltration into skin from 56 Healthy donors and 97 SSc patients. FIGS. 5B-1, 5B-2, 5B-3, and 5B-4 show expression of inflammatory genes such as TNF-α, IL-1β, IL-6, CCL4, and CXCL8 in infiltrating monocytes from blood or skin of SSc patients.

Monocytes isolated from freshly blood of Healthy Donor (HD) or SSc patients were cultured in stiffness at 0 kPA, 0.2 kPA and 50 kPA. RNA were collected after 24 h culture and preformed RNA-sequencing. FIG. 5C shows a PCA plot of RNA-seq analysis. FIG. 5D shows heatmaps of the inflammatory cytokines that are impacted by stiffness. DEGs were analyzed for receptors in HD vs SSc at 0 kPA using QIAGEN Ingenuity Pathway Analysis as shown in FIG. 5E. FIG. 5F shows a Venn diagram of DEGs form HD vs SSc cultured at 0 kPA, 0.2 kPA and 50 kPA. FIG. 5G shows heatmaps of 70 genes from the Venn diagram.

Monocytes were transfected with CRISPR-Cas9 complex targeting for VAV3 using Lipofectamine™ CRISPRMAX reagent. After transfection, monocytes were cultured with baricitinib (1 μM) for 24 h, followed by culturing in fresh media for 48 h, and then culturing at 0.2 kPA stiffness for 6 h. RNA were collected and analyzed for VAV3 and IL-1β expression as shown in FIGS. 5H, 5I, 5J-1, and 5J-2. Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

### Example 6. Gating of Immune Cell Infiltration in Tapes Tripping Mice

8-10 weeks old C57BL/6 mice were either shaved only or tape stripping (TAPE). After day 1, blood and skin biopsies were collected. Infiltration of immune cell CD45 cell, neutrophils (CD45⁺CD11b⁺Ly6G⁺), macrophages (CD45⁺CD11b⁺Ly6G⁻F480⁺), inflammatory monocytes (CD45⁺CD11b⁺Ly6G⁻Ly6C⁺), and infiltrating monocytes (CD45⁺CD11b⁻Ly6G⁻Ly6C⁺CCR2^{low/high}) in inflamed skin were analyzed via flow cytometer as shown in FIG. 6.

### Example 7. Stiffness-Mediated Mechanosensing Trigger Inflammatory Responses in Monocytes

Monocytes from freshly isolated blood from Healthy Donor (HD) were cultured with increasing stiffness. RNA was collected and analyzed for TNF-α, IL-1β, and IL-6 expression. preformed RNA-sequencing as shown in FIGS. 7A-1, 7A-2, and 7A-3.

Monocytes were cultured with increasing stiffness. RNA was collected and preformed RNA-sequencing. FIGS. 7B-1 and 7B-2 show a volcano plot of interferon stimulated genes (ISGs). FIGS. 7C-1 and 7C-2 show a volcano plot of fibrotics genes.

FIGS. 7D-1 and 7D-2 show the expression of genes related to Hippo pathway in human and mice monocytes.

Monocytes were cultured in media alone or with PIEZO1 inhibitor (Dookul: 10 μM) at 0 kPA or 2 kPA for 6 h. Supernatant were collected and analyzed for secretion for TNF-α and IL-6 secretion via ELISA as shown in FIGS. 7E-1 and 7E-2.

Monocytes were cultured in media alone or with PIEZO1activator (Yodal: 20 μM) for 6 h. RNA were collected and analyzed for TNF-α and IL-1β expression as shown in FIGS. 7F-1 and 7F-2.

Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

### Example 8. Stiffness-Mediated Mechanosensing do Require Transcription Factor NFYA but Depend on cAMP Signaling

Monocytes were transfected with CRISPR-Cas9 complex targeting for NFYA on using Lipofectamine™ CRISPRMAX reagent. After transfection, monocytes were cultured for 72 h, followed by 2 kPA stiffness for 6 h. RNA were collected and analyzed for NYFA, TNF-α, and IL-1β expression as shown in FIGS. 8A-1 and 8A-2.

Monocytes were cultured in either 0 kPA or 2 kPA stiffness for 6 h and measured the Ca²⁺ ion flux using flow cytometer as shown in FIGS. 8B-1 and 8B-2.

Monocytes were cultured in media alone or with cAMP inhibitor (KH7: 40 μM) for 1 h, followed by cultured in either 0 kPA or 2 kPA stiffness for 6 h. RNA were collected and analyzed for TNF-α and IL-1β expression as shown in FIGS. 8C-1 and 8C-2.

Monocytes were cultured in media alone or with cAMP activator (Forksolin: 5 μM) for 6 h. RNA were collected and analyzed for TNF-α and IL-1β expression as shown in FIGS. 8D-1 and 8D-2.

Individual donors are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

### Example 9. Inflammation by TAPE Activates Monocytes in Blood and SP1 Inhibition Reduces CD45 Cell Infiltration Mediated by Inflammation

8-10 weeks old C57BL/6 mice were either shaved or tape stripping (TAPE). After day 1, blood was collected for cell sorting to isolate infiltrating monocytes (CD45⁺CD11b⁻Ly6G⁻Ly6C⁺CCR2^{low/high}) RNA were collected from the sorted cells and preformed RNA-sequencing. FIG. 9A shows a volcano plot of inflammatory genes in monocytes from blood compared in between TAPE and Shave. DEGs were analyzed for pathways that are impacted by TAPE using QIAGEN Ingenuity Pathway Analysis as shown in FIG. 9B.

8-10 weeks old C57BL/6 mice were administrated with either PBS or Mithramycin-A (0.5 mg/kg) for 3 consecutive days via intraperitoneal (IP) injection. Then mice were preformed either shaved or tape stripped (TAPE). After 1 day of TAPE, blood and skin biopsies were collected for flow cytometer and analyzed the infiltration of CD45⁺ cells as shown in FIGS. 9C-1 and 9C-2.

Individual mice are indicated; all results are represented as mean±SEM; and statistical significance was evaluated using Mann-Whitney U test or one-way ANOVA, Tukey's multiple comparisons test. ns, P>0.05; *, P<0.05; ***, P<0.001.

### Example 10. Mechanosensing Activates Distinct Genes in Infiltrating Monocytes of SSc to Promote Chronic Inflammation

Monocytes isolated from freshly blood of Healthy Donor (HD) or SSc patients were cultured in stiffness at 0 kPA, 0.2 kPA and 50 kPA. RNA were collected after 24 h culture and preformed RNA-sequencing. FIG. 10A shows heatmaps of fibrotic genes that are impacted by stiffness. DEGs were analyzed for pathway in HD vs SSc at 0 kPA using QIAGEN Ingenuity Pathway Analysis as shown in FIG. 10B. DEGs were analyzed for receptors (FIG. 10C) and transcriptional factor (FIG. 10D) that are impacted by stiffness using QIAGEN Ingenuity Pathway Analysis.

### Example 11. Skin Microenvironment Triggers an Inflammatory Response by Skin-Infiltrating Monocytes

Monocytes play essential roles in inflammation during wound healing and an increase in monocyte recruitment to inflamed sites is well-documented in disease states^{6-8,11,12,24,25}. To characterize the monocyte response in the skin, we used a mild injury model caused by tape stripping, has been described by the inventors and others to trigger inflammation in the skin in both humans and mice²⁶⁻³². This model was selected as it induces a transient inflammatory response associated with resolution after few days, allowing us to study the dynamics of inflammation between acute and chronic states. It also promotes the rapid infiltration by immune cells in the skin, hence allowing to characterize how monocytes are impacted after migrating from the bloodstream to the skin. Consistent with previous findings^{26,27}, tape stripping induced at day 1 the rapid and robust expression of inflammatory genes including TNF-α, IL-6, IL-1α, IL-1β, and IP-10 in the tape-stripped skin (FIGS. 11A-1, 11A-2, 11A-3, 11A-4, and 11A-5). This response was then dampened at day 3 and day 5 (FIGS. 17A and 17B). This was accompanied by the infiltration of CD45+ immune cells (FIG. 111B) and more specifically of neutrophils (CD11b⁺Ly6G⁺) (FIG. 11C) but also of macrophages (CD11b⁺Ly6G⁻F4/80⁺) (FIG. 11D), as described^{26,27}. Furthermore, a substantial increase in Ly6C⁺ inflammatory monocytes^{8,10-12} (CD11b⁺Ly6G⁻Ly6C⁺) infiltrating the inflamed skin was observed, while their number was slightly reduced in the blood (FIG. 11E). The migration of monocytes to sites of inflammation is primarily orchestrated by the CCR2 receptor^{7,33} Indeed, the data herein shows that blood monocytes (CD11b⁺Ly6G⁻Ly6C⁺) expressed low CCR2 levels, while tissue-infiltrating monocytes (CD11b⁺Ly6G⁻Ly6C⁺) could express either high or low levels of CCR2 (FIG. 11F). At 24 h following tape-stripping, a slight decrease in the percentage of Ly6C⁺CCR2^{low} inflammatory monocytes in the blood of mice was noted, while an increase of Ly6C⁺CCR2^{low} and Ly6C⁺CCR2^{high} inflammatory monocytes infiltrating the skin of tape-stripped mice was evident (FIG. 11G), suggesting the migration of inflammatory monocytes from the bloodstream to the skin. The different subsets of skin-infiltrating monocytes were then FACS-sorted and demonstrated increased expression of CD86 on both CCR2^{low} and CCR2^{high} populations as compared to the blood counterparts (FIG. 11H). A significant induction of inflammatory genes such as TNF-α, IL-1b, IL-6, and CXCL2 in skin-infiltrating monocytes was also observed, as compared to blood monocytes (FIGS. 11I-1, 11I-2, 11I-3, and 11I-4). CCR2 was used as a marker of infiltrating cells but recent binding of CCL2 can provoke the internalization of CCR2 during the chemoattraction process. This may explain why little difference between the CCR2^{low} and high populations in the skin was seen. These data show that monocytes are activated once they migrate to the skin following injury, suggesting that the tissue microenvironment regulates skin-infiltrating monocytes by triggering an inflammatory response.

### Example 12. Skin Microenvironment Induces Mechanosensing in Skin-Infiltrating Monocytes

To understand what controls the inflammatory response induced by the skin microenvironment, bulk RNA-sequencing was performed on FACS-sorted monocyte subsets from both the bloodstream and the skin following tape-stripping. In the PCA plot, monocytes were impacted by the tape stripping, even when purified from the blood, whereas skin-infiltrating monocytes were significantly differentiated from blood monocytes with little change between the CCR2^{low} and CCR2^{high} subsets (FIG. 12A). The impact of tape stripping on monocytes in the blood was first tested, and among the various pathways induced by tape stripping was the neutrophil extracellular trap signaling pathway, inducing CCR1, CXCL2, PLA2G3, and TLR2/4 (FIGS. 18C and 18D). This suggests that neutrophils and macrophages likely interact in the skin. Furthermore, the activation of metabolic pathways was observed, particularly those associated with glycolysis and the pentose phosphate pathway (PKM, P6GD, PGD), as well as activation of other pathways such as the unfolded protein response (CALR, DNAJA1), autophagy (ATG7/13), and specific cytokines, including CXCL2, IL1F9, and CCL24 (FIGS. 17C and 17D).

As significant induction of CXCL2 in skin-infiltrating monocytes was observed (FIG. 11I), a deeper analysis was conducted within the RNA-seq and a robust induction of a large set of proinflammatory chemokines in skin-infiltrating monocytes compared to their counterparts in the bloodstream was observed (FIG. 12B). This was associated with the induction of a cytokine storm and innate activation pathways such as TLR or IL-6 signaling pathways, attributed to the induced expression of inflammatory cytokines (FIG. 12C). Intriguingly, a pathway for alternative monocyte activation was also observed (FIG. 12C). This led to the hypothesis that the induced expression of inflammatory cytokines/chemokines in skin-infiltrating monocytes might be mediated not only by TLR signaling (classical monocyte activation), but also by additional unknown signals. Considering that the skin contains an ECM that regulates skin stiffness³⁴, but also initiates mechanotransduction (also known as mechanosensing)³⁵, the inventors hypothesized that the induced expression of inflammatory cytokines in infiltrating monocytes may be mediated through skin stiffness-mediated mechanosensing. To test this hypothesis, an overlapped gene analysis of differentially regulated genes (DEGs) in infiltrating monocytes from blood and skin was performed (FIG. 12D). To comprehend the influence of the skin microenvironment on infiltrating monocytes, DEGs affected by inflamed skin microenvironment were specifically chosen, excluding those impacted by tape stripping in blood. This was because tape stripping was already shown to affect monocytes in the bloodstream, as shown in FIG. 17C. Interestingly, the analysis identified 1826 DEGs (FIG. 12D) which on further analysis revealed activation of pathways associated with the ECM formation and signaling (FIG. 12E). Specifically, pathways such as ECM organization, elastic fiber formation, and integrin cell surface interaction were induced (FIG. 12E).

Additionally, the activation of wound healing pathways, alongside the activation of the cytokine storm and TLR signaling was observed (FIG. 12E). This supports the initial data that indicated that skin-infiltrating monocytes participate in both proinflammatory responses and wound healing (FIG. 12C). Next, the aim was to understand the specific transcription factors TFs regulating monocyte activation in the skin. Core co-TFs of mechanotransduction^{35,36} such as YAP1, SMARCA4, and ARID1A, were activated in infiltrating monocytes (FIG. 12F). Additionally, the TFs SMAD3 and CTNNB1, which are known for their involvement in mechanotransduction^{18,37-40}, were also activated (FIG. 12F). Similarly, TFs IRF3, STAT1/3, NFKB1, RELA, FOS, and JUNB, associated with TLR signaling/nucleic acid sensing⁴¹⁻⁴³ were also activated in skin-infiltrating monocytes (FIG. 12F), supporting the observation of the activation of the cytokine storm pathway. Since mechanotransduction is involved in cell migration and adhesion³⁵, an unbiased assessment of all DEGs for functional activation using IPA was conducted, and key functions associated with cell movement in skin-infiltrating monocytes were identified (FIG. 12G). This cell movement signal significantly overlapped with mechanotransduction pathways with over 60% of genes (159 genes out of 251 genes) regulated by co-TFs of mechanotransduction being involved in cell movement (FIG. 12H). Next, genes associated with either or both cell movement and mechanotransduction were analyzed (FIGS. 12I-12K). In skin-infiltrating monocytes, cell movement linked to the activation of chemokines (CCL2/4/6 and CXCL1/7/8) and chemokine receptors (CCR1/3/7) was observed, while cell migration associated ABI3 and cytokines like IFNG were downregulated (FIG. 12I). Furthermore, in the analysis of genes involved in both cell movement and mechanotransduction, genes such as END1 and CCN1, responsive to mechanotransduction or shear forces^{18,44}, were induced in infiltrating monocytes from the skin. Additionally, genes related to fatty acid metabolism, including FASN and ALXO12, were downregulated in the same context (FIG. 12J). Looking at genes involved in mechanotransduction, transporters, such as SLC43A3 and SLC23A2, were induced, while genes like CHEK2, ACAT2, and TMEM177 are reduced in skin-infiltrating monocytes (FIG. 12K). Finally, to assess the link between mechanosensing and cell movement, it was investigated whether mechanosensing is directly associated with the inflammatory response and wound healing. In the gene overlapping analysis, it was observed that 23 out of 125 genes related to the inflammatory response are regulated by mechanosensing (FIG. 17E). Similarly, it was observed that 22 out of 77 genes from the wound healing pathway are regulated by mechanosensing (FIG. 17F).

In summary, these findings suggest that skin stiffness induces mechanosensing, influencing the function of infiltrating monocytes in the skin for both inflammation and wound healing processes.

### Example 13. High Stiffness Triggers an Inflammatory Response in Human Monocytes

To determine the effect of stiffness on human monocytes, cells were cultured on hydrogel substrates which are crosslinked to create varying stiffness (0 kPA, 0.2 kPA, 2 kPA, and 50 kPA) and are coated with type I collagen to mimic stiffness of skin ECM containing type I collagen 34. These levels of stiffness were selected to model the different environments to which migrating cells are exposed when reaching tissues. The stiffness of the skin is of 1-2 kPA, while during skin fibrosis, the stiffness of the skin can reach up to 25-50 kPA²¹⁻²³. When human monocytes were cultured in these conditions, higher stiffness led to increased secretion and expression of inflammatory cytokines (IL-1β, TNF-α, and IL-6) (FIGS. 13A-13C), along with increased expression of CD86 (FIG. 13D). Next, RNA-sequencing was performed to understand the impact of mechanosensing on global cytokine profiles and it was observed that stiffness-mediated mechanosensing induces expression of proinflammatory cytokines and inhibited the expression of anti-inflammatory cytokines such as IL-10 (FIG. 13E). In addition, stiffness induced fibrotic genes such as GREM1, IL1RN, TNFSF15, and SPP1 (FIG. 13F) but neither inhibited or had little to no effect on interferon stimulated genes (FIG. 13G). As TLR signaling is important for induction of inflammatory genes and since cells will encounter PAMPs and DAMPs once in the skin^{13,45-47}, it was investigated how stiffness-mediated mechanosensing would impact such responses. Notably, the induction of IL-1β by mechanosensing remained unchanged, irrespective of whether monocytes are activated by TLR4/8 or not (FIG. 13H). However, mechanosensing exacerbated TLR4- or TLR8-induced responses with the strong induction of inflammatory genes such as IL-6 and TNF-α (FIGS. 131 and 13J). These findings demonstrate that stiffness triggers mechanosensing-induced signaling which activates monocytes and also amplifies the response by the cells to TLR4 and TLR8 agonists.

### Example 14. Stiffness-Mediated Mechanosensing Activates the Transcription Factor SP1 to Modulate Inflammatory Genes in Human Monocytes

As noted in Example 13, mechanosensing triggered by stiffness possesses the capability to exert transcriptional control over monocyte responses (FIG. 13E), thus it was investigated which TF may be at play. First, previous studies have established that the ECM can trigger mechanosensing signaling in parenchymal cells via the TFs YAP1 and TAZ, which triggers the Hippo pathway³⁵. Although the presence of TAZ and LATS1/2 in primary monocytes could be detected, the analysis revealed the absence of key genes of the Hippo pathway (i.e., TEAD1/2/3/4 and YAP1) in monocytes from both human and mice (FIGS. 18A-1 and 18A-2). By performing a re-analysis of a published sc-RNA-seq dataset from PBMCs and skin samples obtained from a large set of SSc patients⁵⁹, the absence of YAP1 and of all 4 TEADs in inflammatory monocytes from blood and skin of healthy donors and SSc patients was confirmed (FIG. 18B). Furthermore, despite cyclic pressure's known initiation of PIEZO1 signaling in bone marrow-derived macrophages (BMDM)¹⁸, activating or inhibiting PIEZO1 signaling had no impact on stiffness-induced response and the role of PIEZO1 may be more involved in the presence of additional TLR signaling (FIGS. 18C, 18D-1, and 18D-2). This suggests that these key pathway^{16-18,35,48,49} have little role in the activation of human monocytes by mechanosensing, and therefore it was hypothesized that the activation of monocytes involves a previously unexplored TF associated with this process. To delve into this hypothesis, human monocytes were cultured under conditions of 0.2 kPA and 50 kPA stiffness, followed by ATAC-sequencing to evaluate chromatin accessibility throughout the entire genome. Upon plotting the results on a PCA plot, the two different stiffness levels segregated (FIG. 14A) and induced a higher intensity of a large set of peaks at the chromatin level (FIG. 14B). To scrutinize the motifs influenced by the stiffness alteration, De novo motif analysis using HOMER was conducted. This analysis brought to light several motifs, such as FRA1 and NYFA, which exhibit activation in response to stiffness (FIG. 14C). A transcriptomic analysis was then conducted using the RNA-seq data, focusing on TFs. It was observed that numerous TFs are both activated and inhibited by stiffness (FIG. 14D). From both analyses, it appears that the TFs NFYA and SP1 emerged as significantly activated by high stiffness and correlated with each other. These TFs are known to interact and function in concert under normal homeostatic conditions⁵⁰. SP1 was previously demonstrated to regulate IL-10 expression in the RAW264.7 macrophage cell line^{51,52}, and the transcriptional analysis revealed that SP1 is a prominent regulator of the majority of DEGs influenced by stiffness (FIG. 14E). Furthermore, the activation of SP1 in infiltrating monocytes within the skin in the mice under tape-stripping was observed (FIG. 12F), supporting a role for SP1 acting as a key TF involved in mechanosensing-induced signaling in monocytes. Next, the motif peaks associated with these two TFs NFYA and SP1 was evaluated, at varying stiffness levels (0.2 kPA and 50 kPA) (FIGS. 14F-1 and 14F-2). Notably, heightened stiffness levels exhibited more pronounced peaks for both TFs. From these findings, it was hypothesized that mechanosensing is initiated through the involvement of SP1 TFs and used the CRISPR-Cas9 technique to knock down the expression of this TF (FIGS. 14G-1, 14G-2, and 14G-3). Clearly, the reduced expression of SP1, even partial, was enough to hinder the induced expression of IL-1β and TNF-α in response to mechanosensing (FIGS. 14G-1, 14G-2, and 14G-3). Similarly, using a specific inhibitor of SP1 (mithramycin-A)^{53,54}, it was confirmed that blocking SP1 leads to reduced expression of inflammatory genes in response to mechanosensing (FIGS. 14H-1 and 14H-2). Based on these findings using two complementary approaches, it was postulated that SP1 binds to the promoter of these inflammatory genes, inducing their expression. To assess this, we conducted CUT&RUN assays on monocytes cultured under differing stiffness levels (0.2 kPA or 50 kPA). Notably, stiffness-mediated mechanosensing amplified the binding of SP1 to the promoter regions of IL-1β and TNF-α, coinciding with chromatin opening as identified by ATACseq (FIG. 14I). Additionally, chromatin opening in the promoter region of IL-10, induced by mechanosensing, led to the loss of SP1 binding in that region (FIG. 14I), suggesting that SP1 might recruit repressive elements to the opened chromatin in response to mechanosensing. These observations are consistent with the RNA-seq data (FIG. 13E), where mechanosensing induced the expression of IL-1β and TNF-αwhile suppressing the expression of IL-10.

In summary, these findings underscore that mechanosensing activates the TF SP1 to induce the expression of inflammatory cytokines by monocytes.

### Example 15. SP1-Mediated Mechanosensing in Infiltrating Monocytes Promotes Inflammation

Given the observation of SP1 activation by stiffness (FIGS. 14A-14I), the expression levels of genes (such as GSN, SLC39A8, and CDKN1A) known to be regulated by SP1 activation^{55,56} were quantified and it was observed that these were significantly induced in skin-infiltrating high CCR2 monocytes (FIG. 15A). This observation aligns with human data, where these genes are also induced with increasing stiffness (FIG. 18E). To test the role of SP1 in controlling monocyte activation in the skin, mice were treated with mithramycin-A, a specific inhibitor of SP1 that has been well characterized in vitro and in vivo^{53,54}. Then injury was induced by tape stripping, and the impact of SP1 inhibition on overall skin inflammation was explored by evaluating the expression of inflammatory genes in the skin of mice. It was observed that SP1 inhibition significantly decreased the expression of inflammatory genes such as IL-1β, IL-6, IP-10, and TNF-α (FIGS. 15B-1, 15B-2, 15B-3, and 15B-4). In addition, we also observed reduction in the infiltration of CD45+ cells was observed (FIG. 15C). The next goal was to test whether SP1 inhibition in infiltrating monocytes is involved in the overall inflammatory status of the skin. There is no mouse strain allowing for the selective depletion of SP1 in monocytes, as the SP1-floxed mice have not been generated. Therefore, infiltrating monocytes subsets from both blood and skin were FACS-sorted. A reduction in the expression of inflammatory cytokines such as IL-1β, IL-6, and CXCL2 was observed, although not of TNF-α, in skin-infiltrating monocytes (FIGS. 15D-1 and 15D-2). The reduction in CXCL2 expression following SP1 inhibition in skin infiltrating monocytes was intriguing. Given that neutrophils infiltrate the skin during the initiation of inflammation and promote inflammation^{26,27} and that CXCL2 acts as a chemoattractant for neutrophils to the site of inflammation^{57,58}, the impact of SP1 inhibition on neutrophil infiltration was analyzed. A significant reduction in the infiltration of neutrophils in the skin of tape stripped mice with SP1 inhibitor was observed (FIGS. 15E-1, 15E-2, 15E-3, and 15E-4). Together, these findings suggest that skin stiffness initiates SP1-mediated mechanosensing and promotes inflammation.

### Example 16. Mechanosensing Hyperactivated Monocytes from Patients with SSc by Dampening the Inhibitory Feedback Loop Mechanism

The findings disclosed herein established a connection between skin stiffness and the initiation of inflammatory responses once monocytes infiltrated the skin. To determine the relevance of this data to disease, a re-analysis of a published sc-RNA-seq dataset from PBMCs and skin samples obtained from a large set of SSc patients⁵⁹ was performed. The re-analysis revealed an increase in the number of inflammatory monocytes in the skin of SSc patients as compared to HDs (FIG. 16A). This infiltration of monocytes in the skin of patients with SSc was associated with the induction of inflammatory genes such as IL-1β, CXCL2, CXCL8, CXCL16, and CCL3 in infiltrating monocytes, as compared to their counterparts from the blood (FIG. 16B). The induction of inflammatory genes such as IL-1β, CXCL2 and CXCL8 in the skin was strongly linked to the infiltrating monocytes, even though dendritic cells also slightly contributed to the response (FIGS. 19A-1, 19A-2, and 19A-3). These data suggest that monocytes infiltrating the skin can mount an inflammatory response to the skin microenvironment. To determine whether monocytes from SSc patients respond to mechanosensing in a similar fashion to cells from HDs, cultures of monocytes from the blood of HDs and patients with SSc were performed at varying stiffness levels, including 0 kPA, 0.2 kPA and 50 kPA, and conducted transcriptomic analysis. PCA analysis demonstrated a shift in the profile of monocytes from patients with SSc, as cells from patients cultured at 0 kPA (i.e., blood stiffness) resembled cells from HDs but at 0.2 kPA (FIG. 16C). Similarly, cells from patients with SSc cultured at 0.2 kPA were similar to HDs monocytes cultured at 50 kPA (FIG. 16C). These data suggested that monocytes from patients had higher response to mechanosensing signaling. Changes in the expression of inflammatory cytokines by comparing cells from HDs cultured at 0 kPA and 0.2 kPA or from patients at 0 kPA were further studied. Clusters 3 and 4 exhibited similar inflammatory cytokine patterns in monocytes from HDs at 0.2 kPA and from SSc patients at 0 kPA. Cluster 1 showed contrasting patterns, including for IL-10 which appeared lower in HDs at 0.2 kPA (FIG. 16D). The expression of TLRs and other surface receptors which are known to be induced on monocytes in SSc^{13,60,61} were then analyzed. Interestingly, TREM1 receptor activation at 0 kPA in SSc patients' monocytes was found (FIG. 16E). Since TREM1 is known to activate macrophages and induce cytokine production, hence the data suggest a potential role for TREM1 receptor in the exacerbated response of monocytes from SSc patients to mechanosensing. Notably, the transcriptional analysis also revealed activation of fibrotic signaling pathways (FIG. 19B). Furthermore, mechanosensing in SSc monocytes was observed that mirrors the activation pattern seen in HD monocytes for transcription factor SP1 (FIG. 19C). Additionally, activated MSP-RON signaling was observed in SSc monocytes (FIG. 19D), which promotes inflammatory signaling in macrophages^{62,63}. To assess the role of mechanosensing in chronic SSc monocyte activation, overlapped gene analysis was conducted between monocytes from HDs and SSc patients at varying stiffness levels (FIG. 16F). The focus was on identifying mechanosensing-regulated genes shared among all three groups and those uniquely impacted in SSc monocytes, potentially contributing to chronic inflammatory response under fibrotic conditions (i.e., 50 kPA). Through k-Mean clustering of these 606 unique genes, we found cluster-1 genes upregulated and cluster-3 genes downregulated in SSc patients compared to HDs after mechanosensing (FIG. 16G). Interestingly, it was also observed that cluster-4 genes are gradually upregulated with higher stiffness in HDs but downregulated in SSc patients (FIGS. 16G, 16J, 16K-1, and 16K-2). These cluster-4 genes are associated with anti-inflammatory, wound healing, and anti-fibrotic responses. For instance, VAV3 and GJB2 play critical roles in wound healing^{64,65} GPR84 promotes anti-fibrotic pathways⁶⁶, and LRP8 supports anti-inflammatory responses by inhibiting the M2 marker⁶⁷. Additionally, genes like SLC39A8, related to anti-inflammatory responses⁶⁸, induced by mechanosensing in healthy donors, were downregulated in SSc patients (FIGS. 16H and 16I). To further support the conclusion on dysregulated genes by mechanosensing in SSc monocytes, those genes were checked in the RNA-seq from the skin of tape-stripped mice where the inflammation is resolved along with an activated wound healing process as shown in FIG. 12C. Interestingly, the human data showed the induction by mechanosensing of VAV3 and GRP84 expression in monocytes which is something we also observed in skin-infiltrating monocytes in TAPE mice (FIG. 20). To enhance the robustness of the analysis, additional investigations into one of these genes, VAV3, was conducted. Considering VAV3's established role in wound healing⁶⁴, activation of wound healing process and its link to mechanotransduction in skin infiltrating monocytes (FIGS. 12E and 17F), and impaired wound healing in SSc-afflicted skin^{69,70}, CRISPR-Cas9 technique was employed to knockdown VAV3 in human monocytes to mimic the downregulation observed in SSc fibrotic stiffness (FIG. 16J). Decreased VAV3 expression exacerbated the production of inflammatory cytokines like IL-13 and CXCL8 in cells from HDs cultured at 0.2 kPA (FIGS. 16K-1 and 16K-2).

In summary, these findings reveal that mechanosensing promotes inflammatory responses and resolution in healthy individuals, but also suppresses negative feedback mechanisms, including wound healing and anti-fibrotic responses, in SSc monocytes, thereby promoting chronic inflammation.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A method of inhibiting mechanosensing in monocytes, said method comprising inhibition of the SP1-mediated mechanosensing pathway.

2. The method of claim 1, wherein the inhibition of the SP1-mediated mechanosensing pathway via inhibition of SP1 transcription factor, antagonism of the TREM1 receptor, or antagonism of the GPR30 receptor.

3. The method of claim 2, wherein j the inhibition of SP1 transcription factor is by exposure to an SP1 transcription factor inhibitor: 2) the antagonism of the TREM1 receptor is by exposure to a TREM1 receptor antagonist: or 3) the antagonism of the GPR30 receptor is by exposure to a GPR30 receptor antagonist.

4. The method of claim 3, wherein the SP1 transcription factor inhibitor is selected from the group consisting of mithramycin-A, Withaferin A, an analog of mithramycin, and EC-8042.

5.-9. (canceled)

10. The method of claim 3, wherein the TREM1 receptor antagonist is selected from the group consisting of nangibotide, VJDT, and LP17 inhibitory peptide.

11.-14. (canceled)

15. The method of claim 3, wherein the GPR30 receptor antagonist is selected from the group consisting of G15, fulvestrant, fulvestrant-d3, and G36.

16.-18. (canceled)

19. The method of claim 1, wherein the monocytes are in skin tissue.

20. The method of claim 1, wherein the monocytes are human monocytes.

21. The method of claim 1, wherein inhibiting mechanosensing reduces inflammatory cytokines TNF-a, IL-1b, and/or IL-6.

22. The method of claim 21, wherein the inflammatory cytokines are reduced in infiltrating monocytes in inflamed skin.

23. A method of treating a disease or disorder associated with inhibiting mechanosensing in monocytes, said method comprising inhibition of the SP1-mediated mechanosensing pathway.

24. The method of claim 23, wherein the inhibition of the SP1-mediated mechanosensing pathway is via inhibition of SP1 transcription factor, antagonism of the TREM1 receptor, or antagonism of the GPR30 receptor.

25. The method of claim 24, wherein 1 the inhibition of SP1 transcription factor is by exposure to an SP1 transcription factor inhibitor: 2) the antagonism of the TREM1 receptor is by exposure to a TREM1 receptor antagonist: or 3) the antagonism of the GPR30 receptor is by exposure to a GPR30 receptor antagonist.

26. The method of claim 25, wherein the SP1 transcription factor inhibitor is selected from the group consisting of mithramycin-A, Withaferin A, an analog of mithramycin, and EC-8042.

27.-31. (canceled)

32. The method of claim 25, wherein the TREM1 receptor antagonist is selected from the group consisting of nangibotide, VJDT, and LP17 inhibitory peptide.

33.-36. (canceled)

37. The method of claim 25, wherein the GPR30 receptor antagonist is selected from the group consisting of G15, fulvestrant, fulvestrant-d3, and G36.

38.-40. (canceled)

41. The method of claim 23, wherein the disease or disorder is selected from the group consisting of an autoimmune disorder, er a fibrotic disorder, and a skin disease or disorder.

42. (canceled)

43. The method of claim 41, wherein the fibrotic disorder is fibrosis.

44. The method of claim 41, wherein the autoimmune disorder is systemic lupus erythematosus (SLE).

45. The method of claim 41, wherein the skin disease or disorder is selected from the group consisting of systemic sclerosis, psoriasis, scleroderma, cutaneous lupus, chronic cutaneous lupus (discoid lupus), cutaneous lupus erythematosus (CLE), dermatomyositis, Behcet's Disease, ocular cicatricial pemphigoid (OCP), pemphigus, epidermolysis bullosa, epidermolysis bullosa acquisita (EBA), bullous pemphigoid, lichen planus, lichen sclerosus, and a wound.

46.-61. (canceled)


SEQUENCE LISTING (28 sequences)

SEQ ID NO: 1 - DNA, 20 bp, Homo sapiens
  source 1..20: mol_type = other DNA
atcaagaaggtggtgaagca

SEQ ID NO: 2 - DNA, 21 bp, Homo sapiens
  source 1..21: mol_type = other DNA
gtcgctgttgaagtcagagga

SEQ ID NO: 3 - DNA, 20 bp, Homo sapiens
  source 1..20: mol_type = other DNA
tacccccaggagaagattcc

SEQ ID NO: 4 - DNA, 20 bp, Homo sapiens
  source 1..20: mol_type = other DNA
gccatctttggaaggttcag

SEQ ID NO: 5 - DNA, 20 bp, Homo sapiens
  source 1..20: mol_type = other DNA
cttctgcctgctgcactttg

SEQ ID NO: 6 - DNA, 18 bp, Homo sapiens
  source 1..18: mol_type = other DNA
ctgggccagagggctgat

SEQ ID NO: 7 - DNA, 22 bp, Homo sapiens
  source 1..22: mol_type = other DNA
ttcgacacatgggataacgagg

SEQ ID NO: 8 - DNA, 21 bp, Homo sapiens
  source 1..21: mol_type = other DNA
tttttgctgtgagtcccggag

SEQ ID NO: 9 - DNA, 27 bp, Homo sapiens
  source 1..27: mol_type = other DNA
gctctagaatgacttccaagctggccg

SEQ ID NO: 10 - DNA, 27 bp, Homo sapiens
  source 1..27: mol_type = other DNA
cgggatccttatgaattctcagccctc

SEQ ID NO: 11 - DNA, 20 bp, Homo sapiens
  source 1..20: mol_type = other DNA
catacaggcgagaggccatt

SEQ ID NO: 12 - DNA, 20 bp, Homo sapiens
  source 1..20: mol_type = other DNA
gtgcctctgtagctcatccg

SEQ ID NO: 13 - DNA, 20 bp, Homo sapiens
  source 1..20: mol_type = other DNA
ccaaccctggtatgctggag

SEQ ID NO: 14 - DNA, 21 bp, Homo sapiens
  source 1..21: mol_type = other DNA
cctgtgcctcacaaggtaagt

SEQ ID NO: 15 - DNA, 22 bp, Mus musculus
  source 1..22: mol_type = other DNA
ggtctgggccatagaactgatg

SEQ ID NO: 16 - DNA, 20 bp, Mus musculus
  source 1..20: mol_type = other DNA
gccaccacgctcttctgtct

SEQ ID NO: 17 - DNA, 25 bp, Mus musculus
  source 1..25: mol_type = other DNA
aaaccgtttttccatcttcttcttt

SEQ ID NO: 18 - DNA, 17 bp, Mus musculus
  source 1..17: mol_type = other DNA
gacggcacacccaccct

SEQ ID NO: 19 - DNA, 22 bp, Mus musculus
  source 1..22: mol_type = other DNA
gaggataccactcccaacagac

SEQ ID NO: 20 - DNA, 22 bp, Mus musculus
  source 1..22: mol_type = other DNA
aagtgcatcatcgttgttcata

SEQ ID NO: 21 - DNA, 20 bp, Mus musculus
  source 1..20: mol_type = other DNA
gcttccctatggccctcatt

SEQ ID NO: 22 - DNA, 18 bp, Mus musculus
  source 1..18: mol_type = other DNA
gacggtccgctgcaactg

SEQ ID NO: 23 - DNA, 19 bp, Mus musculus
  source 1..19: mol_type = other DNA
ccaaccaccaggctacagg

SEQ ID NO: 24 - DNA, 19 bp, Mus musculus
  source 1..19: mol_type = other DNA
gcgtcacactcaagctctg

SEQ ID NO: 25 - DNA, 19 bp, Mus musculus
  source 1..19: mol_type = other DNA
tgcaccaccaactgcttag

SEQ ID NO: 26 - DNA, 19 bp, Mus musculus
  source 1..19: mol_type = other DNA
ggatgcagggatgatgttc

SEQ ID NO: 27 - DNA, 23 bp, Homo sapiens
  source 1..23: mol_type = genomic DNA
ccatcaacggtctggaactgtgg

SEQ ID NO: 28 - DNA, 23 bp, Homo sapiens
  source 1..23: mol_type = genomic DNA
acggcctgttgtgagacgtttgg